(19) United States
(12) Patent Application Publication
(10) Pub. No.: US 2014/0106398 A1
(43) Pub. Date: Apr. 17, 2014

(54) VECTOR-HOST SYSTEM

(71) Applicants: Roelof Ary Lans Bovenberg, Echt (NL); Jan Andries Kornelis Willem Kiel, Groningen (NL); Thibaut José Wenzel, Echt (NL); Alrik Pieter Los, Echt (NL)

(72) Inventors: Roelof Ary Lans Bovenberg, Echt (NL); Jan Andries Kornelis Willem Kiel, Groningen (NL); Thibaut José Wenzel, Echt (NL); Alrik Pieter Los, Echt (NL)

(73) Assignee: DSM IP ASSETS B.V., Heerlen (NL)

(21) Appl. No.: 14/004,671

(22) PCT Filed: Mar. 12, 2012
(86) PCT No.: PCT/EP12/54302

§ 371 Date: Dec. 16, 2013

(30) Foreign Application Priority Data
Mar. 11, 2011 (EP) 11157871.2

(51) Int. Cl. C12N 15/80

(52) U.S. Cl. CPC C12N 15/80
USPC 435/43; 435/254.11; 435/256.1; 435/256.3; 435/256.7; 435/471; 506/10; 435/91.1; 435/128; 435/158; 435/69.1; 435/101; 435/69.6; 435/69.4; 435/69.3; 435/183; 435/67; 435/166; 435/134; 435/140; 435/139; 435/145; 435/126; 435/161; 435/159

(57) ABSTRACT

The present invention relates to a host cell deficient in an essential gene, comprising a vector, said vector comprising at least said essential gene and an autonomous replication sequence, wherein the host cell is a filamentous fungal cell. The invention also relates to a host cell deficient in an essential gene, comprising a vector, said vector comprising at least said essential gene and an autonomous replication sequence, wherein the host cell comprises a recombinant polynucleotide construct comprising a polynucleotide encoding a biological compound of interest or a compound involved in the synthesis of a biological compound of interest.

### FIELD OF THE INVENTION

The present invention relates to a vector-host system, a host cell, a method for the production of a vector-host system, a method for the production of a biological compound of interest, a method for screening for a polynucleotide encoding a biological compound of interest and a method for use of the host cell and of the vector-host system.

### BACKGROUND OF THE INVENTION

In prokaryotes plasmids, circular DNA molecules that replicate autonomously independent from the host genome, have been the workhorses in both fundamental and biotechnological studies aimed to understand cellular processes, or to produce commercially interesting products such as enzymes, metabolites etc. However, unlike some naturally occurring plasmids, constructed plasmids in bacteria are inherently unstable and require selection (e.g. an auxotrophic marker, a dominant growth marker or an antibiotic resistance marker) to be retained in the cell at a satisfactory level. Consequently, the presence of a plasmid in the cell dictates the medium composition or requires continuous use of (expensive) antibiotics. This feature is not only true for prokaryotes, but also for the limited number of eukaryotes, mainly yeast species and a few filamentous fungi, where autonomously replicating plasmids have so far been utilized with some success. In the budding yeast Saccharomyces cerevisiae plasmid vectors either contain the replicon of the naturally occurring 2 μM plasmid or an autonomously replicating sequence (ARS) isolated from chromosomal DNA. Naturally occurring plasmid origins and organism-specific ARS sequences have also been used as plasmid replicons in some other yeast species. However, in filamentous fungi the approaches used to develop autonomously replicating plasmid vectors in yeast have met with little success, as exemplified by the observation that the 2 μM replicon does not function in these organisms. Consequently, only limited use is made of replicating plasmids in filamentous fungi.

The most utilized plasmid replicon in the ascomycete Aspergillus nidulans that has been shown to function also in other Aspergilli, various Penicillium species as well as in Gibberella fujikuroi and Trichoderma reesei, is the AMA1 replicon (Gems et al., 1991 Gene. 98(1):61-7). This replicon represents a rearranged chromosomal A. nidulans DNA fragment that enables plasmids carrying an auxotrophic marker (like pyrG—uracil) or a dominant marker (like amdS—N-source or bie^{R}—phleomycine) to replicate at multiple copies per chromosome (Fierro et al., 1996 Curr Genet. 29(5):482-9). However, similar to bacterial plasmids, AMA 1-based vectors are mitotically notoriously unstable, and growth on rich media (in case of pyrG and amdS) or growth on antibiotic-free medium (in case of bie^{R}) results in rapid loss of the plasmid from the cells. In many cases, industrial fermentations exploit non-selective complex media that do not allow the use of such mitotically unstable plasmids. As a consequence, in most production systems employing filamentous fungi, expression cassettes have routinely been ectopically integrated into the host genome, or were occasionally inserted via targeted integration (e.g. at the niaD locus allowing selection on chlorate resistance). The recent availability of mutants deficient in orthologs of the mammalian KU70 and KU80 proteins involved in non-homologous end joining (NHEJ) has enabled highly efficient targeted integration strategies in many filamentous fungi (Ninomiya et al., 2004; Proc Natl Acad Sci USA. 101:12248-53). However, ultimate expression remains locus-dependent and the identification of loci that allow efficient expression time-consuming.

It would thus be very advantageous if stabilization of autonomously replicating plasmids in industrial host cells could be enhanced such that non-selective, antibiotic-free media can be used.

### SUMMARY OF THE INVENTION

According to the present invention, there is provide a host cell deficient in an essential gene, comprising a vector, said vector comprising at least said essential gene and an autonomous replication sequence, wherein the host cell is a filamentous fungal cell.

The invention also provides a host cell deficient in an essential gene, comprising a vector, said vector comprising at least said essential gene and an autonomous replication sequence, wherein the host cell comprises a recombinant polynucleotide construct comprising a polynucleotide encoding a biological compound of interest or a compound involved in the synthesis of a biological compound of interest.

A vector-host system comprising a host cell of the invention is also provided by the invention.

The invention further provides a method for the production of a host cell or of a vector-host system, which method comprises:
a. providing a host cell and a vector, which comprises at least a gene essential for said host cell and an autonomous replication sequence,
b. co-transforming the host cell with the vector and a disruption construct for said essential gene to render the host cell deficient in the essential gene.

The invention also provides:

a method for the production of a biological compound of interest comprising culturing the vector-host system or the host cell of the invention under conditions conducive to the production of the biological compound of interest and optionally isolating the compound of interest from the culture broth;
a method for the production of a biological compound of interest comprising producing a host cell or a vector-host system of the invention and culturing such a vector-host system or host cell under conditions conducive to the production of the biological compound of interest and optionally isolating the compound of interest from the culture broth;

a method for the production of a biological compound of interest comprising:

a. providing a host cell, said host cell being deficient in an essential gene, said host cell comprising a vector, said vector comprising at least said essential gene and an autonomous replication sequence, wherein the host cell is (i) a filamentous fungal cell or (ii) a host cell which comprises a recombinant polynucleotide construct comprising a polynucleotide encoding a biological compound of interest or a compound involved in the synthesis of a biological compound of interest,

b. optionally providing said host cell with a recombinant polynucleotide construct comprising a polynucleotide encoding a biological compound of interest or compound involved in the synthesis of a biological compound of interest,

c. culturing the host cell under conditions conducive to the production of the biological compound of interest, and optionally

d. isolating the biological compound of interest from the culture broth;

a method for the production of a biological compound of interest comprising:

a. providing a host cell, said host cell being deficient in an essential gene, said host cell comprising a vector, said vector comprising at least said essential gene and an autonomous replication sequence, wherein said host cell is prepared according to the method of the invention,

b. optionally providing said host cell with a recombinant polynucleotide construct comprising a polynucleotide encoding a biological compound of interest or compound involved in the synthesis of a biological compound of interest,

c. culturing the host cell under conditions conducive to the production of the biological compound of interest, and optionally

d. isolating the biological compound of interest from the culture broth;

a method for screening for a polynucleotide encoding a biological compound of interest comprising:

a. providing a library of polynucleotides possibly containing a polynucleotide encoding a biological compound of interest,

b. providing a multiplicity of individual host cells, said host cell being deficient in an essential gene and comprising a vector, said vector comprising at least said essential gene and an autonomous replication sequence, wherein said host cell is (i) a filamentous fungal cell or (ii) a host cell which comprises a recombinant polynucleotide construct comprising a polynucleotide encoding a biological compound of interest or a compound involved in the synthesis of a biological compound of interest,

c. screening the transformants for expression of the biological compound of interest;

a method for screening for a polynucleotide encoding a biological compound of interest comprising:

a. providing a library of polynucleotides possibly containing a polynucleotide encoding a biological compound of interest,

b. providing a multiplicity of individual host cells, said host cell being deficient in an essential gene and comprising a vector, said vector comprising at least said essential gene and an autonomous replication sequence, wherein said host cell is prepared according to the method of the invention,

c. screening the transformants for expression of the biological compound of interest;
use of the vector-host system according to the invention for the production of a biological compound of interest;
use of the vector-host system according to the invention for expression of a polynucleotide encoding a compound of interest;
use of a host cell deficient in an essential gene to stabilize a vector, said vector comprising at least said essential gene and an autonomous replication sequence; and
use of a host cell to stabilize a vector-host system, said host cell being a host cell prepared according to the method of the invention.

### BRIEF DESCRIPTION OF THE FIGURES

FIG. 1 P. chrysogenum/E. coli shuttle plasmid pDSM-JAK-109, comprising the AMA1 region as derived from pAMPF21*, a constitutively expressed DsRed.SKL cassette and a phleomycin-resistance cassette.

FIG. 2 plasmid pDSM-JAK-105, comprising a recombination cassette that enables replacement of the promoter of the P. chrysogenum tif35 gene by the nitrate-inducible P. chrysogenum niiA promoter as well as the P. chrysogenum niaD gene that is used as selection marker.

FIG. 3 A) pDSM-JAK-106, comprising a recombination cassette that enables to replace the genomic P. chrysogenum tif35 gene including its regulatory regions by an amdS expression cassette. The niaD-F1 and niaD-F2 regions flanking the amdS expression cassette represent a direct repeat that can be used to remove the marker from the genome again using fluoroacetamide counterselection.

B) the P. chrysogenum tif35 deletion cassette of plasmid pDSM-JAK-106.

FIG. 4 plasmid pDSM-JAK-108 comprising the AMA1 region as derived from pAMPF21*, the DsRed.SKL expression cassette and the P. chrysogenum tif35 expression cassette. Note that pDSM-JAK-108 comprises no significant homology to A. niger and P. chrysogenum (when lacking the tif35 gene) chromosomal DNA sequences.

FIG. 5 plasmid pDSM-JAK-116, comprising a recombination cassette that enables integration of the P. chrysogenum tif35 gene with its own regulatory sequences at the niaD locus in the P. chrysogenum genome. Selection is based on absence of nitrate reductase activity that causes resistance towards chlorate.

FIG. 6 plasmid pDSM-JAK-206, pDONR221 derivative containing the A. nidulans tef promoter and the A. nidulans trpC terminator regions.

FIG. 7 plasmid pDSM-JAK-117, pMK-RQ derivative containing the synthetic codon pair-optimized T. reesei cbh1 cDNA.

FIG. 8 plasmid pDSM-JAK-120 comprising the AMA1 region of plasmid pAMPF21*, the P. chrysogenum tif35 expression cassette and the codon pair-optimized T. reesei cbh1 expression cassette.

FIG. 9 the A. niger tif35 deletion cassette. Upon double homologous recombination (illustrated by x) between the fragment and the genomic DNA, the Anig.tif35 gene (including part of the upstream promoter region) was effectively replaced by the amdS selection marker.

FIG. 10 depicts the pEBA1001 vector. Part of the vector fragment was used in bipartite gene-targeting method in combination with the pEBA1002 vector with the goal to delete the ReKu80 ORF in Rasamsonia emersonii. The vector comprises a 2500 bp 5′ upstream flanking region, a lox66 site, the 5′ part of the ble coding sequence driven by the A. nidulans gpdA promoter and the backbone of pUC19 (Invitrogen, Breda, The Netherlands). The E. coli DNA was removed by digestion with restriction enzyme NotI, prior to transformation of the R. emersonii strains.

FIG. 11 depicts the pEBA1002 vector. Part of the vector fragment was used in bipartite gene-targeting method in combination with the pEBA1001 vector with the goal to delete the ReKu80 ORF in Rasamsonia emersonii. The vector comprises the 3′ part of the ble coding region, the A. nidulans trpC terminator, a lox71 site, a 2500 bp 3′ downstream flanking region of the ReKu80 ORF, and the backbone of pUC19 (Invitrogen, Breda, The Netherlands). The E. coli DNA was removed by digestion with restriction enzyme NotI, prior to transformation of the R. emersonii strains.

FIG. 12 depicts a map of pEBA513 for transient expression of cre recombinase in fungi. pEBA513 is a pAMPF21 derived vector containing the AMA1 region and the CAT chloramphenicol resistance gene. Depicted are the cre recombinase gene (cre) expression cassette, containing the A. niger glaA promoter (Pgla), cre recombinase coding region, and niaD terminator. In addition, the hygromycin resistance cassette consisting of the A. nidulans gpdA promoter (PgpdA), hygB coding region and the P. chrysogenum penDE terminator is indicated.

FIG. 13 depicts the strategy used to delete the ReKu80 gene of R. emersonii. The vectors for deletion of ReKu80 comprise the overlapping non-functional ble selection marker fragments (split marker) flanked by loxP sites and 5′ and 3′ homologous regions of the ReKu80 gene for targeting (1). The constructs integrate through triple homologous recombination (X) at the genomic ReKu80 locus and at the overlapping homologous non-functional ble selection marker fragment (2) and replaces the genomic ReKu80 gene copy (3). Subsequently, the selection marker is removed by transient expression of cre recombinase leading to recombination between the lox66 and lox71 sites resulting in the deletion of the ble gene with a remainder double-mutant lox72 site left within the genome (4). Using this overall strategy, the ReKu80 ORF is removed from the genome.

FIG. 14 depicts the pEBA1007 vector. Part of the vector fragment was used in bipartite gene-targeting method in combination with the pEBA1008 vector with the goal to delete the ReTif35 ORF in Rasamsonia emersonii. The vector comprises a 1500 bp 5′ upstream flanking region of the ReTif35 ORF, a lox66 site, the 5′ part of the ble coding region driven by the A. nidulans gpdA promoter and the backbone of pUC19 (Invitrogen, Breda, The Netherlands). The E. coli DNA was removed by digestion with restriction enzyme NotI, prior to transformation of the R. emersonii strains.

FIG. 15 depicts the pEBA1008 vector. Part of the vector fragment was used in bipartite gene-targeting method in combination with the pEBA1007 vector with the goal to delete the ReTif35 ORF in Rasamsonia emersonii. The vector comprises the 3′ part of the ble coding region, the A. nidulans trpC terminator, a lox71 site, a 1500 bp 3′ downstream flanking region of the ReTif35 ORF, and the backbone of pUC19 (Invitrogen, Breda, The Netherlands). The E. coli DNA was removed by digestion with restriction enzyme NotI, prior to transformation of the R. emersonii strains.

FIG. 16 depicts the strategy used to delete the ReTif35 gene of R. emersonii. The vectors for deletion of ReTif35 comprise the overlapping non-functional ble selection marker fragments (split marker) flanked by loxP sites and 5′ and 3′ homologous regions of the ReTif35 gene for targeting (1). The constructs integrate through triple homologous recombination (X) at the genomic ReTif35 locus and at the overlapping homologous non-functional ble selection marker fragment (2) and replaces the genomic ReTif35 gene copy (3). Essential gene Pchr•tif35 is expressed from pDSM-JAK-108 (4).

FIG. 17 depicts the pDSM-JAK-133 vector, an E. coli/S. cerevisiae shuttle vector, which contains the Scer•HIS3 auxotrophic marker, the ARS/CEN replicon and the DsRed.SKL gene expressed by the Scer•TDH3 promoter.

FIG. 18 depicts the pDSM-JAK-134 vector, a derivative of pDSM-JAK-133 in which the entire Scer•HIS3 auxotrophic marker was replaced by the Scer•TIF35 gene.

FIG. 19 depicts the pDSM-JAK-135 vector, a derivative of pDSM-JAK-133 in which part of the Scer•HIS3 auxotrophic marker was replaced by the Scer•TIF35 gene.

FIG. 20 depicts the pDSM-JAK-136 vector comprising the AMA1 region as derived from pAMPF21*, the DsRed.SKL expression cassette and the A. nidulans tif35 gene with its own regulatory sequences. Note that pDSM-JAK-136 comprises no significant homology to A. niger, P. chrysogenum and R. emersonii chromosomal DNA sequences (except aur1).

FIG. 21 depicts the pDSM-JAK-139 vector comprising a recombination cassette that enables to replace the genomic P. chrysogenum aur1 gene by an amdS expression cassette. The niaD-F1 and niaD-F2 regions flanking the amdS expression cassette represent a direct repeat that can be used to remove the marker from the genome again using fluoroacetamide counterselection.

### DETAILED DESCRIPTION OF THE INVENTION

It has now surprisingly been demonstrated that a vector-host system can be produced where the stability of the vector comprising an autonomous replication sequence is substantially increased.

Accordingly, the present invention relates in a first aspect to a vector-host system wherein the host is deficient in an essential gene and wherein the vector comprises at least said essential gene and an autonomous replication sequence.

One great advantage of the system according to the present invention is that it provides for a reliable and versatile system which has increased stability compared to state of the art vector host systems under all relevant conditions, i.e. during sporulation, germination and industrial production processes. Another advantage is that it is possible to work with one vector in various different hosts. Yet another advantage is that it can be used with all kinds of culture media, including complex (also referred to as undefined) medium. All these advantages make it a very versatile system.

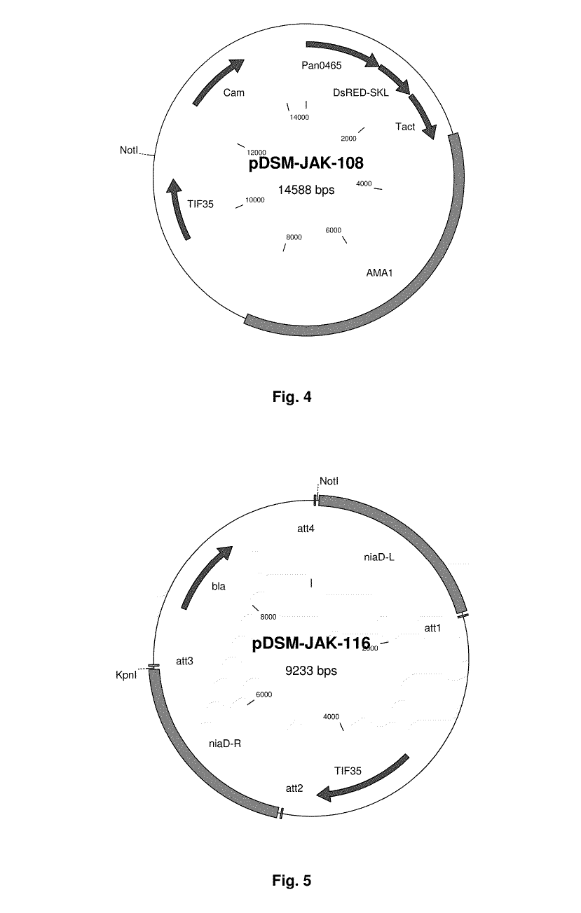

The vector may be any vector (e.g. a plasmid or a virus), which can be conveniently subjected to recombinant DNA procedures. The choice of the vector will typically depend on the compatibility of the vector with the host cell into which the vector is to be introduced. Preferably, the vector is a plasmid. The vector may be a linear or a closed circular plasmid. The vector may further comprise a, preferably non-selective, marker that allows for easy determination of the vector in the host cell. Suitable markers include GFP and DsRed. The chance of gene conversion or integration of the vector into the host genome must be minimized. The person skilled in the art knows how to construct a vector with minimal chance of integration into the genome. In one embodiment, the vector lacks significant similarity with the genome of the host to minimize the chance of integration into the host genome. This may be achieved by using control sequences, such as promoters and terminators, which originate from another species than the host species. In one embodiment, control sequences from A. nidulans are used for a vector which is used in a vector-host system in fungi, in particular a filamentous fungus other than A. nidulans. A suitable example of such a vector is plasmid pDSM-JAK-108 as presented in FIG. 4 of this application. The vectors of the vector-host system of the present invention are also encompassed by the present invention.

Deficiency of a host cell is herein defined as a phenotypic feature, wherein the cell produces less of the product encoded by the essential gene, has a reduced expression level of the mRNA transcribed from the essential gene or has a decreased specific (protein) activity of the product encoded by the essential gene and combinations of these possibilities, as compared to the parent cell which is not deficient in the essential gene. The host cell is typically deficient in view of a genetic lesion in an essential gene resulting in partial or complete non-functionality of the essential gene

Clearly, a host cell with a complete deficiency in an essential gene cannot exist. Thus, a host cell of the invention having a complete deficiency in an essential gene is one which can exist only when it harbours a vector of the invention.

A host cell of the invention thus carries a modification in its genome such that the host cell is partially or completely non-functional for an essential gene and a vector comprising a functional essential gene and autonomous replication sequence. That is to say, the invention provides a a host cell being changed in its genome or endogenous genetic traits/DNA resulting reduced- or non-functionality of an essential gene and a vector comprising a functional essential gene and autonomous replication sequence.

Deficiency can be measured using any assay available to the skilled person, such as transcriptional profiling, Northern blotting, Southern blotting and Western blotting.

Deficiency of the host cell deficient in the essential gene is preferably measured relative to the parent cell that is not deficient in the essential gene. Preferably, the deficiency of the host cell, wherein said host cell produces at least 10% less of the product encoded by the essential gene, has an at least 10% reduced expression level of the mRNA transcribed from the essential gene or has an at least 10% decreased specific (protein) activity of the product encoded by the essential gene as compared to the parent cell which is not deficient in the essential gene. More preferably, the deficiency is at least 20%, even more preferably at least 30%, even more preferably at least 40%, even more preferably at least 50%, even more preferably at least 60%, even more preferably at least 70%, even more preferably at least 75%, even more preferably at least 80%, even more preferably at least 85%, even more preferably at least 90%, even more preferably at least 95%, even more preferably at least 96%, even more preferably at least 97%, even more preferably at least 98%, even more preferably at least 99%, even more preferably at least 99.5%, even more preferably at least 99.9% and most preferably the deficiency is complete, i.e. 100%.

The vector-host system according to the invention has increased stability of the vector comprising an autonomous replication sequence. The stability is preferably measured relative to a vector-host system, wherein the vector is identical but the host is not deficient in the essential gene. The stability is preferably determined comparing the loss of the vector in subsequent cycles of sporulation and single colony isolation on plates with non-selective solid medium. The higher the stability, the longer it will take before the vector is lost from the host cell, in particular on non-selective solid medium, such as complex or undefined medium. In the system according to the invention, the vector is maintained for at least four subsequent cycles of sporulation. Preferably, the vector is maintained for at least five, at least six, at least seven, at least eight, at least nine or at least ten subsequent cycles of sporulation. More preferably, the vector is maintained for at least 15, at least 20, at least 25, at least 30, at least 40, at least 50, at least 60 or at least 70 subsequent cycles of sporulation. If the vector comprises a non-selective colour marker such as GFP or DsRed, presence of the vector in the host can easily be observed by presence of the colour of the marker in the colonies.

Preferably, the increase in stability of the vector-host system according to the invention compared to a vector-host system wherein the vector is identical but the host cell is not deficient in the essential gene is at least a two-fold increase, more preferably at least a three-fold increase, more preferably at least a five-fold increase, more preferably at least a ten-fold increase, more preferably at least a twenty-fold increase, more preferably at least a fifty-fold increase, more preferably at least a hundred-fold increase, more preferably at least a two-hundred-fold increase, more preferably at least a five hundred-fold increase and most preferably at least a thousand-fold increase.

The deficient host cell is preferably obtained by modification of the essential gene in the genome of the parent host cell. That is to say, deficient host cell of the invention is a cell which is modified in its genome so that is partially or completely non-functional for an essential gene.

Said modification of the essential gene in the genome of the host cell is herein defined as any event resulting in a change or addition in the polynucleotide sequence in the cell in the genome of the cell. A modification is construed as one or more modifications. Modification may be accomplished by the introduction (insertion), substitution or removal (deletion) of one or more nucleotides in a nucleotide sequence. This modification may for example be in a coding sequence or a regulatory element required for the transcription or translation of the polynucleotide. For example, nucleotides may be inserted or removed so as to result in the introduction of a stop codon, the removal of a start codon or a change or a frame-shift of the open reading frame of a coding sequence. The modification of a coding sequence or a regulatory element thereof may be accomplished by site-directed or random mutagenesis, DNA shuffling methods, DNA reassembly methods, gene synthesis (see for example Young and Dong, (2004), Nucleic Acids Research 32, Gupta et al. (1968), Proc. Natl. Acad. Sci. USA, 60: 1338-1344; Scarpulla et al. (1982), Anal. Biochem. 121: 356-365; Stemmer et al. (1995), Gene 164: 49-53), or PCR generated mutagenesis in accordance with methods known in the art. Examples of random mutagenesis procedures are well known in the art, such as for example chemical (NTG for example) mutagenesis or physical (UV for example) mutagenesis. Examples of directed mutagenesis procedures are the QuickChange™ site-directed mutagenesis kit (Stratagene Cloning Systems, La Jolla, Calif.), the The Altered Sites® II in vitro Mutagenesis Systems' (Promega Corporation) or by overlap extension using PCR as described in Gene. 1989 Apr. 15; 77(1):51-9. (Ho S N, Hunt H D, Horton R M, Pullen J K, Pease L R “Site-directed mutagenesis by overlap extension using the polymerase chain reaction”) or using PCR as described in Molecular Biology: Current Innovations and Future Trends. (Eds. A. M. Griffin and H. G. Griffin. ISBN 1-898486-01-8; 1995 Horizon Scientific Press, PO Box 1, Wymondham, Norfolk, U.K.).

A modification in the genome can be determined by Southern blotting or by comparing the DNA sequence of the modified cell to the sequence of the non-modified cell. Sequencing of DNA and genome sequencing can be done using standard methods known to the person skilled in the art, for example using Sanger sequencing technology and/or next generation sequencing technologies such as Illumina GA2, Roche 454, and the like, as reviewed in Elaine R. Mardis (2008), Next-Generation DNA Sequencing Methods, Annual Review of Genomics and Human Genetics 9: 387-402.

Preferred methods of modification are based on techniques of gene replacement, gene deletion, or gene disruption.

For example, in case of replacement of a polynucleotide, nucleic acid construct or expression cassette, an appropriate DNA sequence may be introduced at the target locus to be replaced. The appropriate DNA sequence is preferably present on a cloning vector. Preferred integrative cloning vectors comprise a DNA fragment, which is homologous to the polynucleotide or has homology to the polynucleotides flanking the locus to be replaced for targeting the integration of the cloning vector to this pre-determined locus. In order to promote targeted integration, the cloning vector is preferably linearized prior to transformation of the cell. Preferably, linearization is performed such that at least one but preferably either end of the cloning vector is flanked by sequences homologous to the DNA sequence (or flanking sequences) to be replaced. This process is called homologous recombination and this technique may also be used in order to achieve (partial) gene deletion or gene disruption.

For example, for gene disruption, a polynucleotide corresponding to the endogenous polynucleotide may be replaced by a defective polynucleotide, that is a polynucleotide that fails to produce a (fully functional) protein. By homologous recombination, the defective polynucleotide replaces the endogenous polynucleotide. It may be desirable that the defective polynucleotide also encodes a marker, which may be used for selection of transformants in which the nucleic acid sequence has been modified.

Alternatively or in combination with other mentioned techniques, a technique based on in vivo recombination of cosmids in E. coli can be used, as described in: A rapid method for efficient gene replacement in the filamentous fungus Aspergillus nidulans (2000) Chaveroche, M-K., Ghico, J-M. and d'Enfert C; Nucleic acids Research, vol 28, no 22. Other techniques which may be used to inactivate an essential gene are known to the skilled person and may be employed, including gene silencing, for example by RNA interference (RNAi) or antisense strategies, in which mRNA is degraded.

In order to increase the efficiency of inactivation of the essential gene when transforming the vector according to the invention simultaneously with the disruption construct for the essential gene, the essential gene is preferably placed in the genome between loxP sites. The Cre/LoxP system has been shown to be functional in various host cells. When Cre is introduced simultaneously with the vector according to the invention, the essential gene will be excised by the Cre/loxP system. Cre may e.g. be introduced as the encoding polynucleotide on a separate vehicle, as active protein, or may be present as encoding sequence on the vector according to the invention. The loxP sites flanking the essential gene may be introduced by methods known in the art, such as gene replacement. For convenient gene replacement, together with the essential gene a selectable marker may be placed between loxP sites in the genome. Such marker will be excised together with the essential gene upon activation of Cre.

Another way by which the efficiency of transformation may be increased is by placing the essential gene in the host genome under the control of an inducible promoter. The inducible promoter may be any inducible promoter suitable for the purpose, be it a chemically or physically induced promoter (such as by temperature or light). The person skilled in the art knows how to select such promoter. In one embodiment, the niiA promoter from Penicillium chrysogenum is used. This promoter is induced by nitrate but is repressed by ammonium. When culturing on ammonium as the sole N-source in the medium, the host is deficient for the essential gene. When culturing on nitrate as the sole N-source in the medium, the host cell is not deficient in the essential gene. In another embodiment, the xlnA promoter from Aspergillus niger is used. This promoter is induced by xylose but is repressed by glucose. When culturing on glucose medium, the host is deficient for the essential gene. When culturing on xylose medium, the host cell is not deficient in the essential gene.

High transformation frequency is a particularly useful improvement when large pools of transformants are required. An example of such an application is the construction of expression libraries. A time consuming and labour intensive technique in fungi as much less transformants are obtained compared to well known expression systems such as E. coli. Advantages of preparing expression libraries in the intended production organism is the more reliable up scaling and more relevant (post) translational modifications of the host cell. WO2008/000715 provides an efficient method for high throughput transfection of filamentous fungi which method can be used in combination with the methods of the present invention.

The autonomous replication sequence may be any suitable sequence available to the person skilled in the art that it confers to the plasmid replication that is independent of chromosomal replication. Preferably, the autonomous replication sequence is the AMA1 replicon (Gems et al., 1991 Gene. 98(1):61-7). Telomeric repeats may also result in autonomous replication (In vivo linearization and autonomous replication of plasmids containing human telomeric DNA in Aspergillus nidulans, Aleksenko et al. Molecular and General Genetics MGG, 1998—Volume 260, Numbers 2-3, 159-164, DOI: 10.1007/s004380050881). CEN/ARS sequences from yeast may also be suitable.

The essential gene is preferably a gene that has not been shown to be non-essential. More preferably, the essential gene is a gene whose deficiency renders the host cell non-viable. More preferably, the essential gene is a gene whose deficiency renders the host cell non-viable under all conditions and on any medium, in particular complex (undefined) medium. An essential gene in the context of the present invention may be a gene that renders the host cell non-viable when another (non-essential) gene has been rendered deficient. Preferably, the essential gene is an essential gene in other host cells as well. In one embodiment, the essential gene is a gene which is essential in fungi. Preferably, the essential gene is essential in filamentous fungi, more preferably, the essential gene is essential in the filamentous fungi belonging to Penicillium, Aspergillus and Rasamsonia/Talaromyces. Suitable examples of classes of essential genes include, but are not limited to, genes involved in DNA synthesis & modification, RNA synthesis & modification, protein synthesis & modification, proteasome function, the secretory pathway, cell wall biogenesis and cell division. In the context of the present application, the essential gene is not a auxotrophic marker (such as pyrG), dominant growth marker (such as niaD and amdS) and dominant resistance marker (such as ble). A preferred essential gene is the tif35 gene encoding the g subunit of translation initiation factor 3, which has an ortholog in all eukaryotes. In one embodiment, the tif35 gene encoding the g subunit of translation initiation factor 3 from P. chrysogenum is used as the essential gene. A further preferred essential gene is the A. nidulans aur1 gene encoding the enzyme phosphatidylinositol:ceramide phosphoinositol transferase, which is required for sphingolipid synthesis. Thus, in one embodiment, the aur1 gene encoding the enzyme phosphatidylinositol:ceramide phosphoinositol transferase from A. nidulans is used as the essential gene.

Eukaryotic cells have at least two separate pathways (one via homologous recombination (HR) and one via non-homologous recombination (NHR)) through which nucleic acids (in particular DNA) can be integrated into the host genome. The yeast Saccharomyces cerevisiae is an organism with a preference for homologous recombination (HR). The ratio of non-homologous to homologous recombination (NHR/HR) of this organism may vary from about 0.07 to 0.007.

WO 02/052026 discloses mutants of S. cerevisiae having an improved targeting efficiency of DNA sequences into its genome. Such mutant strains are deficient in a gene involved in NHR (KU70).

Contrary to S. cerevisiae, most higher eukaryotes such as filamentous fungal cells up to mammalian cells have a preference for NHR. Among filamentous fungi, the NHR/HR ratio ranges between 1 and more than 100. In such organisms, targeted integration frequency is rather low.

To improve the efficiency of targeted deletion of the essential gene in the genome, it is preferred that the efficiency of homologous recombination (HR) is enhanced in the host cell of the vector-host system according to the invention.

Accordingly, preferably in the vector-host system according to the invention, the host cell is, preferably inducibly, increased in its efficiency of homologous recombination (HR). The host cell is preferably decreased in its efficiency of non-homologous recombination (NHR). The ratio of non-homologous recombination/homologous recombination (NHR/HR) will typically be decreased in a preferred host cell of the invention.

Since the NHR and HR pathways are interlinked, the efficiency of HR can be increased by modulation of either one or both pathways. Increase of expression of HR components will increase the efficiency of HR and decrease the ratio of NHR/HR. Decrease of expression of NHR components will also decrease the ratio of NHR/HR The increase in efficiency of HR in the host cell of the vector-host system according to the invention is preferably depicted as a decrease in ratio of NHR/HR and is preferably calculated relative to a parent host cell wherein the HR and/or NHR pathways are not modulated. The efficiency of both HR and NHR can be measured by various methods available to the person skilled in the art. A preferred method comprises determining the efficiency of targeted integration and ectopic integration of a single vector construct in both parent and modulated host cell. The ratio of NHR/HR can then be calculated for both cell types. Subsequently, the decrease in NHR/HR ration can be calculated. In WO2005/095624, this preferred method is extensively described.

Host cells having a decreased NHR/HR ratio as compared to a parent cell may be obtained by modifying the parent eukaryotic cell by increasing the efficiency of the HR pathway and/or by decreasing the efficiency of the NHR pathway. Preferably, the NHR/HR ratio thereby is decreased at least twice, preferably at least 4 times, more preferably at least 10 times. Preferably, the NHR/HR ratio is decreased in the host cell of the vector-host system according to the invention as compared to a parent host cell by at least 5%, more preferably at least 10%, even more preferably at least 20%, even more preferably at least 30%, even more preferably at least 40%, even more preferably at least 50%, even more preferably at least 60%, even more preferably at least 70%, even more preferably at least 80%, even more preferably at least 90% and most preferably by at least 100%.

According to one embodiment, the ratio of NHR/HR is decreased by increasing the expression level of an HR component. HR components are well-known to the person skilled in the art. HR components are herein defined as all genes and elements being involved in the control of the targeted integration of polynucleotides into the genome of a host, said polynucleotides having a certain homology with a certain pre-determined site of the genome of a host wherein the integration is targeted.

According to an embodiment, the ratio of NHR/HR is decreased by decreasing the expression level of an NHR component. NHR components are herein defined as all genes and elements being involved in the control of the integration of polynucleotides into the genome of a host, irrespective of the degree of homology of said polynucleotides with the genome sequence of the host. NHR components are well-known to the person skilled in the art. Preferred NHR components are a component selected from the group consisting of the homolog or ortholog for the host cell of the vector-host system according to the invention of the yeast genes involved in the NHR pathway: KU70, KU80, RAD50, MRE11, XRS2, LIG4, LIF1, NEJ1 and SIR4 (van den Bosch et al., 2002, Biol. Chem. 383: 873-892 and Allen et al., 2003, Mol. Cancer. Res. 1:913-920). Most preferred are one of KU70, KU80, and LIG4 and both KU70 and KU80. The decrease in expression level of the NHR component can be achieved using the methods as described herein for obtaining the deficiency of the essential gene.

Since it is possible that decreasing the expression of components involved in NHR may result in adverse phenotypic effects, it is preferred that in the host cell of the vector-host system according to the invention, the increase in efficiency in homologous recombination is inducible. This can be achieved by methods known to the person skilled in the art, for example by either using an inducible process for an NHR component (e.g. by placing the NHR component behind an inducible promoter) or by using a transient disruption of the NHR component, or by placing the gene encoding the NHR component back into the genome.

In order to be able to further engineer the host cell of the vector-host system according to the invention, the deficiency of the host cell of the vector-host system according to the invention may or may not be an inducible deficiency. This can be achieved by methods known to the person skilled in the art, for example by placing the essential gene in the genome of the host cell behind an inducible promoter or by using a transient disruption of the essential gene, or by placing the entire essential gene back into the genome. The inducible promoter may be any inducible promoter suitable for the purpose, be it a chemically or physically induced promoter (such as by temperature or light). The person skilled in the art knows how to select such promoter. In one embodiment, the niiA promoter from Penicillium chrysogenum is used. This promoter is induced by nitrate but is repressed by ammonium. When culturing on ammonium as the sole N-source in the medium, the host is deficient for the essential gene. When culturing on nitrate as the sole N-source in the medium, the host cell is not deficient in the essential gene. In another embodiment, the xlnA promoter from Aspergillus niger is used. This promoter is induced by xylose but is repressed by glucose. When culturing on glucose medium, the host is deficient for in essential gene. When culturing on xylose medium, the host cell is not deficient in the essential gene.

The host cell may be any host cell. The host cell may be a eukaryotic host cell. Preferably, the eukaryotic cell is a mammalian, insect, plant, fungal, or algal cell. Preferred mammalian cells include e.g. Chinese hamster ovary (CHO) cells, COS cells, 293 cells, PerC6 cells, and hybridomas. Preferred insect cells include e.g. Sf9 and Sf21 cells and derivatives thereof. More preferably, the eukaryotic cell is a fungal cell, e.g. a yeast cell, such as Candida, Hansenula, Kluyveromyces, Pichia, Saccharomyces, Schizosaccharomyces, or Yarrowia strain. More preferably a Kluyveromyces lactis, Saccharomyces cerevisiae, Hansenula polymorpha, Yarrowia lipolytica or Pichia pastoris, or a filamentous fungal cell. Most preferably, the eukaryotic cell is a filamentous fungal cell.

“Filamentous fungi” include all filamentous forms of the subdivision Eumycota and Oomycota (as defined by Hawksworth et al., In, Ainsworth and Bisby's Dictionary of The Fungi, 8th edition, 1995, CAB International, University Press, Cambridge, UK). The filamentous fungi are characterized by a mycelial wall composed of chitin, cellulose, glucan, chitosan, mannan, and other complex polysaccharides. Vegetative growth is by hyphal elongation. Filamentous fungal strains include, but are not limited to, strains of Acremonium, Agaricus, Aspergillus, Aureobasidium, Chrysosporium, Coprinus, Cryptococcus, Filibasidium, Fusarium, Geosmithia, Humicola, Magnaporthe, Mucor, Myceliophthora, Neocallimastix, Neurospora, Paecilomyces, Penicillium, Piromyces, Panerochaete, Pleurotus, Rasamsonia, Schizophyllum, Talaromyces, Thermoascus, Thermomyces, Thielavia, Tolypocladium, and Trichoderma.

Preferred filamentous fungal cells belong to a species of an Acremonium, Aspergillus, Chrysosporium, Myceliophthora, Penicillium, Rasamsonia, Talaromyces, Thielavia, Fusarium or Trichoderma genus, and even more preferably a species of Aspergillus niger, Acremonium alabamense, Acremonium chrysogenum, Aspergillus awamori, Aspergillus foetidus, Aspergillus sojae, Aspergillus fumigatus, Talaromyces emersonii, Talaromyces thermophilus, Thermomyces lanuginosus, Thermoascus thermophilus, Thermoascus aurantiacus, Thermoascus crustaceus, Rasamsonia emersonii, Rasamsonia byssochlamyoides, Rasamsonia argillacea, Rasamsonia brevistipitata, Rasamsonia cylindrospora, Aspergillus oryzae, Chrysosporium lucknowense, Fusarium oxysporum, Myceliophthora thermophila, Trichoderma reesei, Thielavia terrestris or Penicillium chrysogenum. Most preferred species are Aspergillus niger or Penicillium chrysogenum. When the host cell is an Aspergillus host cell, the host cell preferably is CBS 513.88, CBS124.903 or a derivative thereof. When the host cell is a Penicillium host cell, the host cell is preferably Penicillium chrysogenum strain NRRL 1951 and Wisconsin 54-1255 and all industrial derivatives, in particular Penicillium chrysogenum strains DS54465 and DS61187. When the host cell belongs to the genus Rasamsonia also known as Talaromyces, more preferably the host cell belongs to the species Talaromyces emersonii also known as Rasamsonia emersonii. When the host cell according to the invention is a Talaromyces emersonii also known as Rasamsonia emersonii host cell, the host cell preferably is CBS 124.902 or a derivative thereof.

The Rasamsonia emersonii (R. emersonii) strains used herein are derived from ATCC16479, which is used as wild-type strain. ATCC16479 was formerly also known as Talaromyces emersonii and Penicillium geosmithia emersonii. Upon the use of the name Rasamsonia emersonii also Talaromyces emersonii is meant. Other strain designations of R. emersonii ATCC16479 are CBS393.64, IFO31232 and IMI116815.

Rasamsonia (Talaromyces) emersonii strain TEC-142 is deposited at CENTRAAL BUREAU VOOR SCHIMMELCULTURES, Uppsalalaan 8, P.O. Box 85167, NL-3508 AD Utrecht, The Netherlands on 1 Jul. 2009 having the Accession Number CBS 124902. TEC-142S is a single isolate of TEC-142.

Such strains are suitable for use in the invention.

Several strains of filamentous fungi are readily accessible to the public in a number of culture collections, such as the American Type Culture Collection (ATCC), Deutsche Sammlung von Mikroorganismen and Zellkulturen GmbH (DSM), Centraalbureau Voor Schimmelcultures (CBS), and Agricultural Research Service Patent Culture Collection, Northern Regional Research Center (NRRL) Aspergillus niger CBS 513.88, Aspergillus oryzae ATCC 20423, IFO 4177, ATCC 1011, ATCC 9576, ATCC14488-14491, ATCC 11601, ATCC12892, P. chrysogenum CBS 455.95, Penicillium citrinum ATCC 38065, Penicillium chrysogenum P2, Talaromyces emersonii CBS 124.902, Acremonium chrysogenum ATCC 36225 or ATCC 48272, Trichoderma reesei ATCC 26921 or ATCC 56765 or ATCC 26921, Aspergillus sojae ATCC11906, Chrysosporium lucknowense C1, Garg 27K, VKM-F 3500 D, ATCC44006 and derivatives thereof.

Preferably, when the host cell is a filamentous fungal host cell, the host cell additionally comprises modifications in its genome such that it is deficient in at least one of glucoamylase (glaA), acid stable alpha-amylase (amyA), neutral alpha-amylase (amyBI and amyBII), oxalic acid hydrolase (oahA), a toxin, such as ochratoxin and fumonisin, and protease transcriptional regulator PrtT. Preferably, the host cell additionally comprises a disruption of the pepA gene encoding the major extracellular aspartic protease PepA.

Preferably, the host cell additionally comprises a modification of Sec61. A preferred Sec61 modification is a modification which results in a one-way mutant of Sec61; i.e. a mutant wherein the de novo synthesized protein can enter the ER via Sec61, but the protein cannot leave the ER via Sec61. Such modifications are extensively described in WO2005/123763. Most preferably, the Sec 61 modification is the S376W mutation in which Serine 376 is replaced by Tryptophan. These and other possible host modifications are also described in WO2012/001169, WO2011/009700, WO2007/062936, WO2006/040312 or WO2004/070022.

The vector-host system according to the invention can conveniently be used for the production of a biological compound of interest. The host cell may already be capable to produce the biological compound of interest. The host cell may also be provided with a recombinant homologous or heterologous polynucleotide construct that encodes a polypeptide involved in the production of the biological compound of interest.

Accordingly, the host cell of the vector-host system according to the invention preferably comprises a recombinant polynucleotide construct comprising a polynucleotide encoding a compound involved in the synthesis of a biological compound of interest. The polynucleotide may also directly encode a biological compound of interest.

The recombinant polynucleotide construct encoding a compound of interest or a polypeptide involved in the synthesis of a biological compound of interest may be located on the vector of the vector-host system according to the invention.

The biological compound of interest according to the invention can be any biological compound. The biological compound may be biomass or any biopolymer or metabolite. The biological compound may be encoded by a single polynucleotide or a series of polynucleotides composing a biosynthetic or metabolic pathway or may be the direct product of a single polynucleotide or may be products of a series of polynucleotides. The biological compound may be native to the host cell or heterologous. The biological compound may be modified according WO2010/102982.

The term “heterologous biological compound” is defined herein as a biological compound which is not native to the cell; or a native biological compound in which structural modifications have been made to alter the native biological compound.

The term “biopolymer” is defined herein as a chain (or polymer) of identical, similar, or dissimilar subunits (monomers). The biopolymer may be any biopolymer. The biopolymer may for example be, but is not limited to, a nucleic acid, polyamine, polyol, polypeptide (or polyamide), or polysaccharide.

The biopolymer may be a polypeptide. The polypeptide may be any polypeptide having a biological activity of interest. The term “polypeptide” is not meant herein to refer to a specific length of the encoded product and, therefore, encompasses peptides, oligopeptides, and proteins. Polypeptides further include naturally occurring allelic and engineered variations of the above-mentioned polypeptides and hybrid polypeptides. The polypeptides may be a modified polypeptide according WO2010/102982. The polypeptide may be native or may be heterologous to the host cell. The polypeptide may be a collagen or gelatin, or a variant or hybrid thereof. The polypeptide may be an antibody or parts thereof, an antigen, a clotting factor, an enzyme, a hormone or a hormone variant, a receptor or parts thereof, a regulatory protein, a structural protein, a reporter, or a transport protein, protein involved in secretion process, protein involved in folding process, chaperone, peptide amino acid transporter, glycosylation factor, transcription factor, synthetic peptide or oligopeptide, intracellular protein. The intracellular protein may be an enzyme such as, a protease, ceramidases, epoxide hydrolase, aminopeptidase, acylases, aldolase, hydroxylase, aminopeptidase, lipase, non-ribosomal synthetase or polyketide synthetase. The polypeptide may be an enzyme secreted extracellularly, such as an oxidoreductase, transferase, hydrolase, lyase, isomerase, catalase, cellulase, chitinase, cutinase, deoxyribonuclease, dextranase, esterase. The enzyme may be a carbohydrase, e.g. cellulases such as endoglucanases, β-glucanases, cellobiohydrolases or β-glucosidases, GH61-enzymes, hemicellulases or pectinolytic enzymes such as xylanases, xylosidases, mannanases, galactanases, galactosidases, pectin methyl esterases, pectin lyases, pectate lyases, endo polygalacturonases, exopolygalacturonases rhamnogalacturonases, arabanases, arabinofuranosidases, arabinoxylan hydrolases, galacturonases, lyases, or amylolytic enzymes; hydrolase, isomerase, or ligase, phosphatases such as phytases, esterases such as lipases, phospholipases, galactolipases, proteolytic enzymes, dairy enzymes and products (e.g. chymosin, casein), oxidoreductases such as oxidases, transferases, or isomerases. The enzyme may be a phytase. The enzyme may be an aminopeptidase, asparaginase, amylase, carbohydrase, carboxypeptidase, endo-protease, metallo-protease, serine-protease, catalase, chitinase, cutinase, cyclodextrin glycosyltransferase, deoxyribonuclease, esterase, alpha-galactosidase, beta-galactosidase, glucoamylase, alpha-glucosidase, beta-glucosidase, haloperoxidase, protein deaminase, invertase, laccase, lipase, mannosidase, mutanase, oxidase, pectinolytic enzyme, peroxidase, phospholipase, polyphenoloxidase, ribonuclease, transglutaminase, or glucose oxidase, hexose oxidase, monooxygenase.

According to the present invention, a polypeptide can also be a fused or hybrid polypeptide to which another polypeptide is fused at the N-terminus or the C-terminus of the polypeptide or fragment thereof. A fused polypeptide is produced by fusing a nucleic acid sequence (or a portion thereof) encoding one polypeptide to a nucleic acid sequence (or a portion thereof) encoding another polypeptide.

Techniques for producing fusion polypeptides are known in the art, and include, ligating the coding sequences encoding the polypeptides so that they are in frame and expression of the fused polypeptide is under control of the same promoter (s) and terminator. The hybrid polypeptides may comprise a combination of partial or complete polypeptide sequences obtained from at least two different polypeptides wherein one or more may be heterologous to the host cell.

The biopolymer may be a polysaccharide. The polysaccharide may be any polysaccharide, including, but not limited to, a mucopolysaccharide (e.g., heparin and hyaluronic acid) and nitrogen-containing polysaccharide (e.g., chitin). In a more preferred option, the polysaccharide is hyaluronic acid.

The polynucleotide of interest according to the invention may encode an enzyme involved in the synthesis of a primary or secondary metabolite, such as organic acids, carotenoids, antibiotics, anti-cancer drug, pigments isoprenoids, alcohols, fatty acids and vitamins. Such metabolite may be considered as a biological compound according to the present invention.

The term “metabolite” encompasses both primary and secondary metabolites; the metabolite may be any metabolite. Preferred metabolites are citric acid, gluconic acid and succinic acid, antibiotics, bioactive drugs, biofuels and building blocks of biomaterials.

The metabolite may be encoded by one or more genes, such as in a biosynthetic or metabolic pathway. Primary metabolites are products of primary or general metabolism of a cell, which are concerned with energy metabolism, growth, and structure. Secondary metabolites are products of secondary metabolism (see, for example, R. B. Herbert, The Biosynthesis of Secondary Metabolites, Chapman and Hall, New York, 1981).

The primary metabolite may be, but is not limited to, an amino acid, carboxylic acid, fatty acid, nucleoside, nucleotide, sugar, triglyceride, or vitamin.

The compounds of interest may be an organic compound selected from glucaric acid, gluconic acid, glutaric acid, adipic acid, succinic acid, tartaric acid, oxalic acid, acetic acid, lactic acid, formic acid, malic acid, maleic acid, malonic acid, citric acid, fumaric acid, itaconic acid, levulinic acid, xylonic acid, aconitic acid, ascorbic acid, kojic acid, coumeric acid, a poly unsaturated fatty acid, ethanol, 1,3-propane-diol, ethylene, glycerol, xylitol, carotene, astaxanthin, lycopene and lutein.

The secondary metabolite may be, but is not limited to, an alkaloid, coumarin, flavonoid, polyketide, quinine, steroid, peptide, or terpene, a β-lactam antibiotic such as Penicillin G or Penicillin V and fermentative derivatives thereof, a cephalosporin, cyclosporin or lovastatin. The secondary metabolite may be an antibiotic, antifeedant, attractant, bacteriocide, fungicide, hormone, insecticide, or rodenticide. Preferred antibiotics are cephalosporins and beta-lactams.

The biological compound of interest may also be the product of a selectable marker. A selectable marker is a product of a polynucleotide of interest which product provides for biocide or viral resistance, resistance to heavy metals, prototrophy to auxotrophs, and the like. Selectable markers include, but are not limited to, amdS (acetamidase), arg B (ornithinecarbamoyltransf erase), bar (phosphinothricinacetyltransferase), hygB (hygromycin phosphotransferase), niaD (nitratereductase), pyrG (orotidine-5′-phosphate decarboxylase), sC (sulfate adenyltransferase), trpC (anthranilate synthase), ble (phleomycin resistance protein), as well as equivalents thereof.

According to one embodiment, the biological compound of interest is preferably a polypeptide as described herein. Preferably, said polypeptide is an enzyme as described herein.

According to another embodiment, the biological compound of interest is preferably a metabolite as described herein.

When the biological compound of interest is a biopolymer as defined earlier herein, the host cell may already be capable to produce the biopolymer. The host cell may also be provided with a recombinant homologous or heterologous polynucleotide construct that encodes a polypeptide involved in the production of the biological compound of interest. The person skilled in the art knows how to modify a microbial host cell such that it is capable of production of the compound involved in the production of the biological compound of interest.

The term “recombinant polynucleotide construct” is herein referred to as a nucleic acid molecule, either single- or double-stranded, which is isolated from a naturally occurring gene or which has been modified to contain segments of nucleic acid which are combined and juxtaposed in a manner which would not otherwise exist in nature. The term recombinant polynucleotide construct is synonymous with the term “expression cassette” when the nucleic acid construct contains all the control sequences required for expression of a coding sequence, wherein said control sequences are operably linked to said coding sequence.

The term “operably linked” is defined herein as a configuration in which a control sequence is appropriately placed at a position relative to the coding sequence of the DNA sequence such that the control sequence directs the production of an mRNA or a polypeptide.

The term “control sequences” is defined herein to include all components, which are necessary or advantageous for the production of mRNA or a polypeptide, either in vitro or in a host cell. Each control sequence may be native or foreign to the nucleic acid sequence encoding the polypeptide. Such control sequences include, but are not limited to, a leader, Shine-Delgarno sequence, optimal translation initiation sequences (as described in Kozak, 1991, J. Biol. Chem. 266:19867-19870), a polyadenylation sequence, a pro-peptide sequence, a pre-pro-peptide sequence, a promoter, a signal sequence, and a transcription terminator. At a minimum, the control sequences include a promoter, and a transcriptional stop signal as well as translational start and stop signals. Control sequences may be optimized to their specific purpose. Preferred optimized control sequences used in the present invention are those described in WO2006/077258.

The control sequences may be provided with linkers for the purpose of introducing specific restriction sites facilitating ligation of the control sequences with the coding region of the nucleic acid sequence encoding a polypeptide.

The control sequence may be an appropriate promoter sequence (promoter).

The control sequence may also be a suitable transcription terminator (terminator) sequence, a sequence recognized by a filamentous fungal cell to terminate transcription. The terminator sequence is operably linked to the 3′-terminus of the nucleic acid sequence encoding the polypeptide. Any terminator, which is functional in the cell, may be used in the present invention.

According to the present invention, control sequences will always be chosen in such a way that the chance of gene conversion or integration of the vector into the host genome is minimized. The person skilled in the art knows how to construct a vector with minimal chance of integration into the genome. In one embodiment, the vector lacks significant similarity with the genome of the host to minimize the chance of integration into the host genome. This may be achieved by using control sequences, such as promoters and terminators, which originate from another species than the host species. In one embodiment, control sequences from A. nidulans are used for a vector which is used in a vector-host system in fungi, in particular a filamentous fungus other than A. nidulans.

Depending on the host, suitable control sequences may be obtained from the polynucleotides encoding A. nidulans trpC, A. nidulans gpdA, A. nidulans ribosomal protein S8 (AN0465), A. nidulans tef (AN4218) A. oryzae TAKA amylase, A. niger glucoamylase (glaA), A. nidulans anthranilate synthase, A. niger alpha-glucosidase, trpC and Fusarium oxysporum trypsin-like protease.

The control sequence may also be a suitable leader sequence (leaders), a non-translated region of an mRNA which is important for translation by the filamentous fungal cell. The leader sequence is operably linked to the 5′-terminus of the nucleic acid sequence encoding the polypeptide. Any leader sequence, which is functional in the cell, may be used in the present invention.

Depending on the host, suitable leaders may be obtained from the polynucleotides encoding A. oryzae TAKA amylase and A. nidulans triose phosphate isomerase and A. niger GlaA and phytase.

Other control sequences may be isolated from the Penicillium IPNS gene, or pcbC gene, the beta tubulin gene. All the control sequences cited in WO 01/21779 are herewith incorporated by reference.

The control sequence may also be a polyadenylation sequence, a sequence which is operably linked to the 3′-terminus of the nucleic acid sequence and which, when transcribed, is recognized by the filamentous fungal cell as a signal to add polyadenosine residues to transcribed mRNA. Any polyadenylation sequence, which is functional in the cell, may be used in the present invention.

The term “promoter” is defined herein as a DNA sequence that binds RNA polymerase and directs the polymerase to the correct downstream transcriptional start site of a nucleic acid sequence encoding a biological compound to initiate transcription. RNA polymerase effectively catalyzes the assembly of messenger RNA complementary to the appropriate DNA strand of a coding region. The term “promoter” will also be understood to include the 5′-non-coding region (between promoter and translation start) for translation after transcription into mRNA, cis-acting transcription control elements such as enhancers, and other nucleotide sequences capable of interacting with transcription factors. The promoter may be any appropriate promoter sequence suitable for a eukaryotic or prokaryotic host cell, which shows transcriptional activity, including mutant, truncated, and hybrid promoters, and may be obtained from polynucleotides encoding extra-cellular or intracellular polypeptides either homologous (native) or heterologous (foreign) to the cell. The promoter may be a constitutive or inducible promoter.

Examples of inducible promoters that can be used are chemically and physically inducible promoters, including starch-, cellulose-, hemicellulose (such as xylan- and/or xylose-inducible), copper-, oleic acid, oxygen and nitrate—inducible promoters. The promoter may be selected from the group, which includes but is not limited to promoters obtained from the polynucleotides encoding P. chrysogenum nitrate or nitrite reductase, A. oryzae TAKA amylase, Rhizomucor miehei aspartic proteinase, A. niger neutral alpha-amylase, A. niger acid stable alpha-amylase, A. niger or A. awamori glucoamylase (glaA), A. niger or A. awamori endoxylanase (xInA) or beta-xylosidase (xInD), R. miehei lipase, A. oryzae alkaline protease, A. oryzae triose phosphate isomerase, A. nidulans acetamidase, Fusarium venenatum amyloglucosidase (WO 00/56900), Fusarium venenatum Dania (WO 00/56900), Fusarium venenatum Quinn (WO 00/56900), Fusarium oxysporum trypsin-like protease (WO 96/00787), Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase IV, Trichoderma reesei endoglucanase V, Trichoderma reesei xylanase I, Trichoderma reesei xylanase II, Trichoderma reesei beta-xylosidase, as well as the NA2-tpi promoter (a hybrid of the promoters from the polynucleotides encoding A. niger neutral alpha-amylase and A. oryzae triose phosphate isomerase), and mutant, truncated, and hybrid promoters thereof. Other examples of promoters are the promoters described in WO2006/092396 and WO2005/100573, which are herein incorporated by reference. Another example of the use of promoters is described in WO2008/098933. Other examples of inducible (heterologous) promoters are the alcohol inducible promoter alcA, the tet system using the tetracycline-responsive promoter, the estrogen-responsive promoter (Pachlinger et al. (2005), Appl & Environmental Microbiol 672-678).

In order to facilitate expression, the polynucleotide encoding the polypeptide involved in the production of the compound of interest may be a synthetic polynucleotide. The synthetic polynucleotides may be optimized in codon use, preferably according to the methods described in WO2006/077258 or WO2008/000632. WO2008/000632 addresses codon-pair optimization. Codon-pair optimization is a method wherein the nucleotide sequences encoding a polypeptide have been modified with respect to their codon-usage, in particular the codon-pairs that are used, to obtain improved expression of the nucleotide sequence encoding the polypeptide and/or improved production of the encoded polypeptide. Codon pairs are defined as a set of two subsequent triplets (codons) in a coding sequence (CDS).

Furthermore, standard molecular cloning techniques such as DNA isolation, gel electrophoresis, enzymatic restriction modifications of nucleic acids, Southern analyses, transformation of cells, etc., are known to the skilled person and are for example described by Sambrook et al. (1989) “Molecular Cloning: a laboratory manual”, Cold Spring Harbor Laboratories, Cold Spring Harbor, N.Y. and Innis et al. (1990) “PCR protocols, a guide to methods and applications” Academic Press, San Diego.

A nucleic acid may be amplified using cDNA, mRNA or alternatively, genomic DNA, as a template and appropriate oligonucleotide primers according to standard PCR amplification techniques. The nucleic acid so amplified can be cloned into an appropriate vehicle and characterized by DNA sequence analysis.

### Host Cell

The present invention further relates to a host cell deficient in an essential gene, comprising a vector, said vector comprising at least said essential gene and an autonomous replication sequence.

The host cell according to the invention is preferably a host cell of the vector-host system as defined earlier herein in the section “vector-host system”.

The essential gene is preferably an essential gene as defined earlier herein in the section “vector-host system”.

The vector is preferably a vector as defined earlier herein in the section “vector-host system”.

Deficiency is defined as earlier herein in the section “vector-host system”.

Deficiency can be measured using any assay available to the skilled person, such as transcriptional profiling, Northern blotting, Southern blotting and Western blotting.

Deficiency of the host cell deficient in the essential gene is preferably measured relative to the parent cell that is not deficient in the essential gene. Preferably, the deficiency of the host cell, wherein said host cell produces at least 10% less of the product encoded by the essential gene and/or has an at least 10% reduced expression level of the mRNA transcribed from the essential gene and/or has an at least 10% decreased specific (protein) activity of the product encoded by the essential gene as compared to the parent cell which is not deficient in the essential gene. More preferably, the deficiency is at least 20%, even more preferably at least 30%, even more preferably at least 40%, even more preferably at least 50%, even more preferably at least 60%, even more preferably at least 70%, even more preferably at least 75%, even more preferably at least 80%, even more preferably at least 85%, even more preferably at least 90%, even more preferably at least 95%, even more preferably at least 96%, even more preferably at least 97%, even more preferably at least 98%, even more preferably at least 99%, even more preferably at least 99.5%, even more preferably at least 99.9% and most preferably the deficiency is complete, i.e. 100%.

The host cell according to the invention has increased stability of the vector comprising an autonomous replication sequence. The stability is preferably measured relative to a host cell, wherein the vector is identical but the host is not deficient in the essential gene. The stability is preferably determined comparing the loss of the vector in subsequent cycles of sporulation and single colony isolation on plates with non-selective solid medium. The higher the stability, the longer it will take before the vector is lost from the host cell, in particular on non-selective solid medium, such as complex or undefined medium. In the system according to the invention, the vector is maintained for at least four subsequent cycles of sporulation. Preferably, the vector is maintained for at least five, at least six, at least seven, at least eight, at least nine or at least ten subsequent cycles of sporulation. More preferably, the vector is maintained for at least 15, at least 20, at least 25, at least 30, at least 40, at least 50, at least 60 or at least 70 subsequent cycles of sporulation. If the vector comprises a non-selective colour marker such as GFP or DsRed, presence of the vector in the host can easily be observed by presence of the colour of the marker in the colonies.

Preferably, the increase in stability of the host cell according to the invention compared to a host cell wherein the vector is identical but the host cell is not deficient in the essential gene is at least a two-fold increase, more preferably at least a three-fold increase, more preferably at least a five-fold increase, more preferably at least a ten-fold increase, more preferably at least a twenty-fold increase, more preferably at least a fifty-fold increase, more preferably at least a hundred-fold increase, more preferably at least a two-hundred-fold increase, more preferably at least a five hundred-fold increase and most preferably at least a thousand-fold increase.

The autonomous replication sequence is preferably one as defined earlier herein in the section “vector-host system”.

In one embodiment, the host cell according to the invention is, preferably inducibly, increased in its efficiency of homologous recombination (HR) as defined earlier herein in the section “vector-host system”. The host cell is preferably decreased in its efficiency of non-homologous recombination (NHR). The ratio of non-homologous recombination/homologous recombination (NHR/HR) will typically be decreased in a preferred host cell of the invention.

Host cells having a decreased NHR/HR ratio as compared to a parent cell may be obtained by modifying the parent eukaryotic cell by increasing the efficiency of the HR pathway and/or by decreasing the efficiency of the NHR pathway. Preferably, the NHR/HR ratio thereby is decreased at least twice, preferably at least 4 times, more preferably at least 10 times. Preferably, the NHR/HR ratio is decreased in the host cell of the vector-host system according to the invention as compared to a parent host cell by at least 5%, more preferably at least 10%, even more preferably at least 20%, even more preferably at least 30%, even more preferably at least 40%, even more preferably at least 50%, even more preferably at least 60%, even more preferably at least 70%, even more preferably at least 80%, even more preferably at least 90% and most preferably by at least 100%.

In order to be able to further engineer the host cell according to the invention, the deficiency of said host cell may or may not be an inducible deficiency. This can be achieved by methods known to the person skilled in the art, for example by placing the essential gene in the genome of the host cell behind an inducible promoter or by using a transient disruption of the essential gene, or by placing the entire essential gene back into the genome. The inducible promoter may be any inducible promoter suitable for the purpose, be it a chemically or physically induced promoter (such as by temperature or light). The person skilled in the art knows how to select such promoter. In one embodiment, the niiA promoter from Penicillium chrysogenum is used. This promoter is induced by nitrate but is repressed by ammonium. When culturing on ammonium as the sole N-source in the medium, the host is deficient for the essential gene. When culturing on nitrate as the sole N-source in the medium, the host cell is not deficient in the essential gene. In another embodiment, the xlnA promoter from Aspergillus niger is used. This promoter is induced by xylose but is repressed by glucose. When culturing on glucose medium, the host is deficient for the essential gene. When culturing on xylose medium, the host cell is not deficient in the essential gene.

The host cell according to the invention can conveniently be used for the production of a biological compound of interest.

Accordingly, the host cell according to the invention preferably comprises a recombinant polynucleotide construct comprising a polynucleotide encoding a compound involved in the synthesis of a biological compound of interest. The polynucleotide may also directly encode a biological compound of interest.

Said recombinant polynucleotide construct encoding a compound of interest may be located on the vector of the vector-host system according to the invention, said recombinant polynucleotide construct may be located on the genome of the host of the vector-host system according to the invention, or said recombinant polynucleotide construct may be located on a separate vehicle.

The biological compound of interest is preferably one as defined earlier herein in the section “vector-host system”.

### Method for the Production of a Vector-Host System

The present invention further relates to a method for the production of a vector-host system according to the invention, said method comprising:
a. providing a host cell and a vector, which comprises at least a gene essential for said host cell and an autonomous replication sequence,
b. co-transforming the host cell with the vector and a disruption construct for said essential gene to render the host cell deficient in the essential gene.

If the host cell is inducibly deficient in the essential gene, a method for the production of a vector-host system according to the invention comprising:
a. providing a host cell and a vector, which vector comprising at least a gene essential for said host cell and an autonomous replication sequence,
b. transforming the host cell with a disruption construct for said essential gene to render the host cell inducibly deficient in the essential gene,
c. transforming the host cell with the vector;
   is also part of the present invention. The skilled person will understand that the host cell produced in step b. cannot be in the ‘deficient state’ as long as the host has not been transformed with the vector in step c.

Additional variations for method of transformation, co-transformation and use of disruption constructs are described in WO2008/000715 (High throughput transfection), WO2009/150195 and WO2008/113847.

Host cell, vector, essential gene and autonomous replication sequence are preferably those as defined earlier herein in the section “vector-host system”. Deficiency is defined as earlier herein in the section “vector-host system”.

Deficiency of the host cell deficient in the essential gene is preferably measured relative to the parent cell that is not deficient in the essential gene. Preferably, the deficiency of the host cell, wherein said host cell produces at least 10% less of the product encoded by the essential gene and/or has an at least 10% reduced expression level of the mRNA transcribed from the essential gene and/or has an at least 10% decreased specific (protein) activity of the product encoded by the essential gene as compared to the parent cell which is not deficient in the essential gene. More preferably, the deficiency is at least 20%, even more preferably at least 30%, even more preferably at least 40%, even more preferably at least 50%, even more preferably at least 60%, even more preferably at least 70%, even more preferably at least 75%, even more preferably at least 80%, even more preferably at least 85%, even more preferably at least 90%, even more preferably at least 95%, even more preferably at least 96%, even more preferably at least 97%, even more preferably at least 98%, even more preferably at least 99%, even more preferably at least 99.5%, even more preferably at least 99.9% and most preferably the deficiency is complete, i.e. 100%.

The host cell has increased stability of the vector comprising an autonomous replication sequence. The stability is preferably measured relative to a host cell, wherein the vector is identical but the host is not deficient in the essential gene. The stability is preferably determined comparing the loss of the vector in subsequent cycles of sporulation and single colony isolation on plates with non-selective solid medium. The higher the stability, the longer it will take before the vector is lost from the host cell, in particular on non-selective solid medium, such as complex or undefined medium. In the system according to the invention, the vector is maintained for at least four subsequent cycles of sporulation. Preferably, the vector is maintained for at least five, at least six, at least seven, at least eight, at least nine or at least ten subsequent cycles of sporulation. More preferably, the vector is maintained for at least 15, at least 20, at least 25, at least 30, at least 40, at least 50, at least 60 or at least 70 subsequent cycles of sporulation. I If the vector comprises a non-selective colour marker such as GFP or DsRed, presence of the vector in the host can easily be observed by presence of the colour of the marker in the colonies.

Preferably, the increase in stability of the host cell compared to a host cell wherein the vector is identical but the host cell is not deficient in the essential gene is at least a two-fold increase, more preferably at least a three-fold increase, more preferably at least a five-fold increase, more preferably at least a ten-fold increase, more preferably at least a twenty-fold increase, more preferably at least a fifty-fold increase, more preferably at least a hundred-fold increase, more preferably at least a two-hundred-fold increase, more preferably at least a five hundred-fold increase and most preferably at least a thousand-fold increase.

Preferably, the host cell is, preferably inducibly, increased in its efficiency of homologous recombination (HR) as described earlier herein in the section “vector-host system”. The host cell is preferably decreased in its efficiency of non-homologous recombination (NHR). The ratio of non-homologous recombination/homologous recombination (NHR/HR) will typically be decreased in a preferred host cell of the invention.

The host cell can be rendered deficient for the essential gene according to the methods described earlier herein in the section “vector-host system”. In one embodiment, the host cell is transformed with a disruption construct. Such disruption construct preferably comprises a polynucleotide corresponding to the wild-type polynucleotide such that the wild-type polynucleotide is replaced by a defective polynucleotide, i.e. a polynucleotide that fails to produce a (fully functional) protein. By homologous recombination, the defective polynucleotide replaces the endogenous polynucleotide.

In one embodiment, the vector comprising at least the essential gene for said host cell and an autonomous replication sequence is transformed simultaneously with the disruption construct in order to simultaneously disrupt the genomic copy of the essential gene and introduce the complementing copy with the vector.

The vector comprising at least the essential gene for the host cell and an autonomous replication sequence may also comprise a recombinant polynucleotide construct encoding a compound involved in the synthesis of a biological compound of interest. The biological compound of interest is preferably one described earlier herein in the section “vector-host system”. Said recombinant polynucleotide construct encoding a compound involved in the synthesis of a biological compound of interest may also be introduced into the host cell on a separate vehicle using a separate transformation event, either before or after disruption of the essential gene and either before or after introduction of the vector comprising at least the essential gene for the host cell and an autonomous replication sequence. In one embodiment of the invention, the cre/loxP system as described earlier herein is used to inactivate the essential gene on the genome.

Transformation of a host cell by introduction of a polynucleotide, an expression vector or a polynucleotide construct into the cell is preferably performed by techniques well known in the art (see Sambrook & Russell; Ausubel, supra). Transformation may involve a process consisting of protoplast formation, transformation of the protoplasts, and regeneration of the cell wall in a manner known per se. Suitable procedures for transformation of Aspergillus, Penicillium and Rasamsonia cells are described in EP 238 023 and Yelton et al., 1984, Proceedings of the National Academy of Sciences USA 81:1470-1474 and Cantoral et al., 1987; Bio/Technol. 5: 494-497. Suitable procedures for transformation of Aspergillus and other filamentous fungal host cells using Agrobacterium tumefaciens are described in e.g. De Groot et al., Agrobacterium tumefaciens-mediated transformation of filamentous fungi. Nat. Biotechnol. 1998, 16:839-842. Erratum in: Nat Biotechnol 1998 16:1074. A suitable method of transforming Fusarium species is described by Malardier et al., 1989, Gene 78:147156 or in WO 96/00787. Other methods can be applied such as a method using biolistic transformation as described in: Christiansen et al., Biolistic transformation of the obligate plant pathogenic fungus, Erysiphe graminis f.sp. hordei. 1995, Curr Genet. 29:100-102. Yeast may be transformed using the procedures described by Becker and Guarente, In Abelson, J. N. and Simon, M. I., editors, Guide to Yeast Genetics and Molecular Biology, Methods in Enzymology, Volume 194, pp 182-187, Academic Press, Inc., New York; Ito et al., 1983, Journal of Bacteriology 153: 163; and Hinnen et al., 1978, Proceedings of the National Academy of Sciences USA 75: 1920.

### Method for the Production of a Biological Compound of Interest

The present invention further relates to a method for the production of a biological compound of interest comprising culturing the vector-host system according to the section “vector-host system” or the host cell according to the section “host cell” under conditions conducive to the production of the biological compound of interest and optionally isolating the compound of interest from the culture broth.

The invention further relates to a method for the production of a biological compound of interest comprising:
a. providing a host cell, said host cell being deficient in an essential gene, said host cell comprising a vector, said vector comprising at least said essential gene and an autonomous replication sequence,
b. optionally providing said host cell with a recombinant polynucleotide construct comprising a polynucleotide encoding a biological compound of interest or a compound involved in the synthesis of a biological compound of interest,
c. culturing the host cell under conditions conducive to the production of the biological compound of interest, and optionally
d. isolating the biological compound of interest from the culture broth.

Host cell, vector, essential gene and autonomous replication sequence are preferably those as defined earlier herein in the section “vector-host system”.

Deficiency is defined as earlier herein in the section “vector-host system”.

Deficiency of the host cell deficient in the essential gene is preferably measured relative to the parent cell that is not deficient in the essential gene. Preferably, the deficiency of the host cell, wherein said host cell produces at least 10% less of the product encoded by the essential gene and/or has an at least 10% reduced expression level of the mRNA transcribed from the essential gene and/or has an at least 10% decreased specific (protein) activity of the product encoded by the essential gene as compared to the parent cell which is not deficient in the essential gene. More preferably, the deficiency is at least 20%, even more preferably at least 30%, even more preferably at least 40%, even more preferably at least 50%, even more preferably at least 60%, even more preferably at least 70%, even more preferably at least 75%, even more preferably at least 80%, even more preferably at least 85%, even more preferably at least 90%, even more preferably at least 95%, even more preferably at least 96%, even more preferably at least 97%, even more preferably at least 98%, even more preferably at least 99%, even more preferably at least 99.5%, even more preferably at least 99.9% and most preferably the deficiency is complete, i.e. 100%.

The host cell has increased stability of the vector comprising an autonomous replication sequence. The stability is preferably measured relative to a host cell, wherein the vector is identical but the host is not deficient in the essential gene. The stability is preferably determined comparing the loss of the vector in subsequent cycles of sporulation and single colony isolation on plates with non-selective solid medium. The higher the stability, the longer it will take before the vector is lost from the host cell, in particular on non-selective solid medium, such as complex or undefined medium. In the system according to the invention, the vector is maintained for at least four subsequent cycles of sporulation. Preferably, the vector is maintained for at least five, at least six, at least seven, at least eight, at least nine or at least ten subsequent cycles of sporulation. More preferably, the vector is maintained for at least 15, at least 20, at least 25, at least 30, at least 40, at least 50, at least 60 or at least 70 subsequent cycles of sporulation. I If the vector comprises a non-selective colour marker such as GFP or DsRed, presence of the vector in the host can easily be observed by presence of the colour of the marker in the colonies.

Preferably, the increase in stability of the host cell compared to a host cell wherein the vector is identical but the host cell is not deficient in the essential gene is at least a two-fold increase, more preferably at least a three-fold increase, more preferably at least a five-fold increase, more preferably at least a ten-fold increase, more preferably at least a twenty-fold increase, more preferably at least a fifty-fold increase, more preferably at least a hundred-fold increase, more preferably at least a two-hundred-fold increase, more preferably at least a five hundred-fold increase and most preferably at least a thousand-fold increase.

Preferably, the host cell is, preferably inducibly, increased in its efficiency of homologous recombination (HR) as described earlier herein in the section “vector-host system”. The host cell is preferably decreased in its efficiency of non-homologous recombination (NHR). The ratio of non-homologous recombination/homologous recombination (NHR/HR) will typically be decreased in a preferred host cell of the invention.

The biological compound of interest is preferably one described earlier herein in the section “vector-host system”.

The host cell can be rendered deficient for the essential gene according to the methods described earlier herein in the section “vector-host system”. In one embodiment, the host cell is transformed with a disruption construct. Such disruption construct preferably comprises a polynucleotide corresponding to the wild-type polynucleotide such that the wild-type polynucleotide is replaced by a defective polynucleotide, i.e. a polynucleotide that fails to produce a (fully functional) protein. By homologous recombination, the defective polynucleotide replaces the endogenous polynucleotide.

In one embodiment, the vector comprising at least the essential gene for said host cell and an autonomous replication sequence is transformed simultaneously with the disruption construct in order to simultaneously disrupt the genomic copy of the essential gene and introduce the complementing copy with the vector.

The host cell may already be capable to produce the biological compound of interest. The host cell may also be provided with a recombinant homologous or heterologous polynucleotide construct that encodes a polypeptide involved in the production of the biological compound of interest.

The vector comprising at least the essential gene for the host cell and an autonomous replication sequence may accordingly comprise a recombinant polynucleotide construct encoding a compound involved in the synthesis of a biological compound of interest. Said recombinant polynucleotide construct encoding a compound involved in the synthesis of a biological compound of interest may also be introduced into the host cell on a separate vehicle using a separate transformation event, either before or after disruption of the essential gene and either before or after introduction of the vector comprising at least the essential gene for the host cell and an autonomous replication sequence. The recombinant polynucleotide construct encoding a compound involved in the synthesis of a biological compound of interest and the vehicle comprising it, is preferably one as defined earlier herein in the section “vector-host system”.

Culturing as used herein means that the microbial cells are cultivated in a nutrient medium suitable for production of the biological compound of interest using methods known in the art. For example, the host cells may be cultivated by shake flask cultivation, small-scale or large-scale fermentation (including continuous, batch, fed-batch, or solid state fermentations) in laboratory or industrial fermentors performed in a suitable medium and under conditions allowing the compound of interest to be produced and, optionally, isolated. The cultivation takes place in a suitable nutrient medium comprising carbon and nitrogen sources and inorganic salts, using procedures known in the art (see, e.g., Bennett, J. W. and LaSure, L., eds., More Gene Manipulations in Fungi, Academic Press, CA, 1991). Suitable media are available from commercial suppliers or may be prepared using published compositions (e.g., in catalogues of the American Type Culture Collection). The system according to the present invention is stable and versatile enough to maintain the vector-host system on all kinds of media, including non-selective, complex media which are typically exploited in industrial fermentations. If the compound of interest is secreted into the nutrient medium, the compound can be isolated directly from the medium. If the compound of interest is not secreted, it can be isolated from cell lysates.

The biological compound of interest may be isolated by methods known in the art. For example, the biological compound of interest may be isolated from the nutrient medium by conventional procedures including, but not limited to, centrifugation, filtration, extraction, spray drying, evaporation, or precipitation. The isolated biological compound of interest may then be further purified by a variety of procedures known in the art including, but not limited to, chromatography (e.g., ion exchange, affinity, hydrophobic, chromatofocusing, and size exclusion), electrophoretic procedures (e.g., preparative isoelectric focusing, differential solubility (e.g., ammonium sulfate precipitation), or extraction (see, e.g., Protein Purification, J.-C. Janson and Lars Ryden, editors, VCH Publishers, New York, 1989). In some applications the biological compound of interest may be used without substantial isolation from the culture broth; separation of the culture medium from the biomass may be adequate.

### Method for Screening for a Polynucleotide Encoding a Biological Compound of Interest

The present invention also relates to a method for screening for a polynucleotide encoding a biological compound of interest comprising:
a. providing a library of polynucleotides possibly containing an polynucleotide encoding a biological compound of interest,
b. providing a multiplicity of individual host cells, said host cell being deficient in an essential gene, comprising a vector, said vector comprising at least said essential gene and an autonomous replication sequence,
c. screening the transformants for expression of the biological compound of interest.

After identification of the transformed host cell comprising the polynucleotide encoding the biological compound of interest in step (c), the polynucleotide may optionally be isolated from the host cell identified. Subsequently, the isolated polynucleotide may be retransformed into a suitable host cell, e.g. for industrial production of the biological compound of interest.

Screening may be performed using detection methods known in the art that are specific for the biological compound of interest. These detection methods include, but are not limited to use of specific antibodies, high performance liquid chromatography, capillary chromatography, electrophoresis, formation of an enzyme product, or disappearance of an enzyme substrate.

Host cell, vector, essential gene and autonomous replication sequence are preferably those as defined earlier herein in the section “vector-host system”.

Deficiency is defined as earlier herein in the section “vector-host system”.

Deficiency of the host cell deficient in the essential gene is preferably measured relative to the parent cell that is not deficient in the essential gene. Preferably, the deficiency of the host cell, wherein said host cell produces at least 10% less of the product encoded by the essential gene and/or has an at least 10% reduced expression level of the mRNA transcribed from the essential gene and/or has an at least 10% decreased specific (protein) activity of the product encoded by the essential gene as compared to the parent cell which is not deficient in the essential gene. More preferably, the deficiency is at least 20%, even more preferably at least 30%, even more preferably at least 40%, even more preferably at least 50%, even more preferably at least 60%, even more preferably at least 70%, even more preferably at least 75%, even more preferably at least 80%, even more preferably at least 85%, even more preferably at least 90%, even more preferably at least 95%, even more preferably at least 96%, even more preferably at least 97%, even more preferably at least 98%, even more preferably at least 99%, even more preferably at least 99.5%, even more preferably at least 99.9% and most preferably the deficiency is complete, i.e. 100%.

The host cell has increased stability of the vector comprising an autonomous replication sequence. The stability is preferably measured relative to a host cell, wherein the vector is identical but the host is not deficient in the essential gene. The stability is preferably determined comparing the loss of the vector in subsequent cycles of sporulation and single colony isolation on plates with non-selective solid medium. The higher the stability, the longer it will take before the vector is lost from the host cell, in particular on non-selective solid medium, such as complex or undefined medium. In the system according to the invention, the vector is maintained for at least four subsequent cycles of sporulation. Preferably, the vector is maintained for at least five, at least six, at least seven, at least eight, at least nine or at least ten subsequent cycles of sporulation. More preferably, the vector is maintained for at least 15, at least 20, at least 25, at least 30, at least 40, at least 50, at least 60 or at least 70 subsequent cycles of sporulation. I If the vector comprises a non-selective colour marker such as GFP or DsRed, presence of the vector in the host can easily be observed by presence of the colour of the marker in the colonies.

Preferably, the increase in stability of the host cell compared to a host cell wherein the vector is identical but the host cell is not deficient in the essential gene is at least a two-fold increase, more preferably at least a three-fold increase, more preferably at least a five-fold increase, more preferably at least a ten-fold increase, more preferably at least a twenty-fold increase, more preferably at least a fifty-fold increase, more preferably at least a hundred-fold increase, more preferably at least a two-hundred-fold increase, more preferably at least a five hundred-fold increase and most preferably at least a thousand-fold increase.

Preferably, the host cell is, preferably inducibly, increased in its efficiency of homologous recombination (HR) as described earlier herein in the section “vector-host system”.

The biological compound of interest is preferably one described earlier herein in the section “vector-host system”.

The host cell can be rendered deficient for the essential gene according to the methods described earlier herein in the section “vector-host system”. In one embodiment, the host cell is transformed with a disruption construct. Such disruption construct preferably comprises a polynucleotide corresponding to the wild-type polynucleotide such that the wild-type polynucleotide is replaced by a defective polynucleotide, i.e. a polynucleotide that fails to produce a (fully functional) protein. By homologous recombination, the defective polynucleotide replaces the endogenous polynucleotide.

In one embodiment, the vector comprising at least the essential gene for said host cell and an autonomous replication sequence is transformed simultaneously with the disruption construct in order to simultaneously disrupt the genomic copy of the essential gene and introduce the complementing copy with the vector.

Transformation is preferably performed as described earlier herein.

The library may encode a biological compound of interest that is native or heterologous to the host cell. The polynucleotide encoding the biological compound of interest may originate from any organism capable of producing the biological compound of interest, including multicellular organisms and microorganisms e.g. bacteria and fungi. The origin of the polynucleotide may also be synthetic meaning that the library could be comprised of e.g. codon optimized variants encoding the same polypeptide or the library could comprise variants obtained by shuffling techniques, or directed evolution techniques known in the art.

The vector-host system and the host cell according to the invention can conveniently be used for the production of a biological compound of interest.

Accordingly, the present invention further relates to the use of the vector-host system or of the host cell according to the invention for the production of a biological compound of interest.

The vector-host system and host cell, are preferably those as defined earlier herein in the sections “vector-host system” and “host cell”. The methods and host cells, vectors, biological compound of interest and other desired features are preferably those as defined earlier herein in the section “Method for the production of a biological compound of interest”.

The vector-host system and the host cell according to the invention can conveniently be used for screening for a polynucleotide encoding a biological compound of interest.

Accordingly, the present invention further relates to the use of the vector-host system or of the host cell according to the invention for screening for a polynucleotide encoding a compound of interest.

The vector-host system and host cell, are preferably those as defined earlier herein in the sections “vector-host system” and “host cell”. The methods and host cells, vectors, biological compound of interest, library and other desired features are preferably those as defined earlier herein in the section “Method for screening for a polynucleotide encoding a biological compound of interest”.

The invention described and claimed herein is not to be limited in scope by the specific embodiments herein disclosed, since these embodiments are intended as illustrations of several aspects of the invention. Any equivalent embodiments and/or combinations of preferred aspects of the invention are intended to be within the scope of this invention. Indeed, various modifications of the invention in addition to those shown and described herein will become apparent to those skilled in the art from the foregoing description. Such modifications are also intended to fall within the scope of the appended claims. In the case of conflict, the present disclosure including definitions will control.

### EXAMPLES

### Strains

P. chrysogenum DS17690, (deposited on 15 Apr. 2008 at the Centraalbureau voor Schimmelcultures, Utrecht, The Netherlands with deposition number CBS122850), is a high penicillin producing strain.

P. chrysogenum DS54465, a derivative of DS17690 wherein the P. chrysogenum KU70 homologue has been deleted (Snoek et al. (2009) Fungal Genetics and Biology 46, 418-426).

P. chrysogenum DS58274, a derivative of DS54465 carrying an inactivated niaD locus with a GFP.SKL expression cassette allowing its use as both auxotropic marker as well as a transformation control.

P. chrysogenum DS61187, a derivative of the much used laboratory strain Wis54-1255 deficient in NHEJ.

A. niger WT 1: This A. niger strain is a CBS513.88 strain comprising a gene deletion of the A. niger KU70 homolog, designated as hdfA. The construction of deletion vector and genomic deletion of the hdfA gene has been described in detail in WO05/095624. The vectors pDEL-HDFA, described in WO05/095624, has been used according the “MARKER-GENE FREE” approach as described in EP 0 635 574 B1. The procedure described above resulted in an hdfA deficient recombinant A. niger CBS 513.88 strain, possessing finally no foreign DNA sequences at all. As such, WT1 has an increased efficiency of homologous recombination and thus a decrease ration of NHR/HR. A. niger strain CBS513.88 was deposited on 10 Aug. 1988 at the Centraalbureau voor Schimmelcultures, Utrecht, The Netherlands.

A. niger WT 2 is a WT 1 strain comprising a deletion of the gene encoding glucoamylase (glaA). WT 2 was constructed by using the “MARKER-GENE FREE” approach as described in EP 0 635 574 B1. In this patent it is extensively described how to delete glaA specific DNA sequences in the genome of CBS 513.88. The procedure resulted in a MARKER-GENE FREE ΔglaA recombinant A. niger CBS 513.88 strain, possessing finally no heterologous DNA sequences at all.

The Rasamsonia emersonii (R. emersonii) strains used herein are derived from ATCC16479, which is used as wild-type strain. ATCC16479 was formerly also known as Talaromyces emersonii and Penicillium geosmithia emersonii. Upon the use of the name Rasamsonia emersonii also Talaromyces emersonii is meant. Other strain designations of R. emersonii ATCC16479 are CBS393.64, IFO31232 and IMI116815.

Rasamsonia (Talaromyces) emersonii strain TEC-142 is deposited at CENTRAAL BUREAU VOOR SCHIMMELCULTURES, Uppsalalaan 8, P.O. Box 85167, NL-3508 AD Utrecht, The Netherlands on 1 Jul. 2009 having the Accession Number CBS 124902. TEC-142S is a single isolate of TEC-142.

Media

(i) YGG medium containing 0.8% KCl, 1.6% glucose, 0.67% Difco yeast nitrogen base (Becton, Dickinson & Co., Sparks, Md., USA), 0.15% citric acid, 0.6 K₂HPO₄, 0.2% yeast extract, pH 6.2, with addition of 100 U/ml penicillin and 100 μg/ml streptomycin (Gibco, Invitrogen, Breda, The Netherlands). YGG-sucrose medium contained in addition 34.2% of sucrose.

P. chrysogenum protoplasts and mycelia were grown on:

(i) phleomycin selection agar containing: 1% Difco yeast nitrogen base), 0.225 citric acid, 0.9% K₂HPO₄, 0.1% yeast extract (Becton, Dickinson & Co.), 2 glucose, 28.7% sucrose and 2% agar, and 1 ml/L of a trace element solution pH 7, supplemented with 100 U/ml penicillin, 100 μg/ml streptomycin and 50 μg/ml phleomycin (Invivogen, San Diego, USA).

(ii) Nitrogen source selection agar contained 0.3% NaCl, 0.05% MgSO₄.7H₂O, 0.001% FeSO₄.7H₂O, 1% glucose, 10 mM potassiumphosphate buffer pH 6.8 and 2% agar and 1 ml/L of a trace element solution and was supplemented with 0.1 acetamide and 15 mM CsCl₂ (acetamide selection agar), 0.1% (NH₄)₂SO₄ (ammonium selection plates) or 0.1% NaNO₃ (nitrate selection plates). For protoplast regeneration 34.2% sucrose was added.

(iii) fluoroacetamide selection agar contained 0.3% NaCl, 0.05% MgSO₄.7H₂O, 0.001% FeSO₄.7H₂O, 1% glucose, 0.1% fluoroacetamide, 5 mM urea, 10 mM potassiumphosphate buffer pH 6.8 and 2% agar.

(iv) chlorate selection agar contained 0.3% NaCl, 0.1% KH₂PO₄, 0.05% MgSO₄.7H₂O, 0.001% FeSO₄.7H₂O, 1% glucose, 0.185% adenine 1.25% KClO₃ and 2% agar and 1 ml/L of a trace element solution pH 6.5. For protoplast regeneration 34.2 sucrose was added.

(v) R agar contained 0.52% v/v glycerol, 0.75% v/v beet molasses, 0.5% yeast extract, 300 mM NaCl, 0.2 m MgSO₄.7H₂O, 0.44 mM KH₂PO₄, 3.3 μM NH₄Fe(SO₄)₂.12H₂O, 0.4 μM CuSO₄.5H₂O, 1.45 mM CaSO₄.2H₂O and 2% agar. When required, NaNO₃ was added to a final concentration of 0.1%.

A. nidulans FGSC A4 spores were isolated from R-agar plates and cultivated on YGG medium.

PDA: Potato Dextrose Agar, Oxoid, non-selective medium, prepared according to the supplier's instructions.

R. emersonii mycelia were grown on PDA or Rasamsonia agar medium. Rasamsonia agar medium contained per liter: 15 g of Salt fraction no. 3, 30 g of cellulose, 7.5 g of Bacto peptone, 15 g of Grain flour, 5 g of KH₂PO₄, 1 g of CaCl₂.2H₂O, 20 g of Bacto agar, pH 6.0. The salt fraction no. 3 was fitting the disclosure of WO98/37179, Table 1. Deviations from the composition of this table were CaCl₂.2H₂O 1.0 g/l, KCl 1.8 g/L, citric acid 1H₂O 0.45 g/L (chelating agent). For spore batch preparation, strains were grown from stocks on Rasamsonia agar medium in 10 cm diameter Petri dishes for 5-7 days at 40° C. Strain stocks were stored at −80° C. in 10% glycerol.

Escherichia coli DH5α was used for cloning purposes. Cells were grown at 37° C. in LB medium (1% Bacto tryptone (Becton, Dickinson & Co.), 0.5% Yeast Extract and 0.5% NaCl) supplemented with 50 μg/ml kanamycin, 100 μg/ml ampicillin, 100 μg/ml carbenicillin or 15 μg/ml chloramphenicol.

### Plasmids

pDONR P4-P1R, pDONR 221 and pDONR P2R-P3 are multisite Gateway vectors; Kan^{R} Cm^{R} from Invitrogen, USA.
pDEST R4-R3 is a multisite Gateway vector; Amp^{R}Cm^{R} from Invitrogen, USA.
pENTR221-niaD_{F1}-amdS-niaD_{F2} is a pDONR221 derivative with a [niaD_{F1}-P_{Anid•gpdA}-Anid•amdS-niaD_{F2}] cassette; Kan^{R} Anid•amdS Laboratory collection
pENTR41-niaD_{L} is pDONR P4-P1R with 5 prime-region of Pchr•niaD; Kan^{R}; DSM lab collection
pENTR23-niaD_{R} is pDONR P2R-P3 with 3 prime-region of Pchr•niaD; Kan^{R} DSM lab collection
pAMPF21 is a P. chrysogenum/E. coli shuttle vector with AMA1 region; Cm^{R} Phleo^{R}; Fierro et al., 1996 Curr Genet. 29(5):482-9.
pAMPF21* is pAMPF21 lacking specific restriction sites (HindIII, Asp718i) in the AMA1 region; Cm^{R}; DSM lab collection
pBBK-001 is an E. coli plasmid containing a [P_{Pchr•pcbC}-DsRed.SKL-T_{Pchr•penDE}] cassette; Amp^{R}; Kiel et al., 2009 Funct Integr Genomics. 9(2):167-84.
pBBK-007 is a plasmid containing a [P_{Anid•gpdA}-DsRed.SKL-T_{Pchr•penDE}] cassette; Amp^{R}; Meijer et al., 2010 Appl Environ Microbiol. 76(17):5702-9.
pENTR221-stuffer is pDONR221 with portion of P. chrysogenum ATG15 gene flanked by suitable restriction sites; Kan^{R}; Laboratory collection
pUG34-DsRed.SKL is an E. coli/S. cerevisiae shuttle vector, which contains the Scer•HIS3 auxotrophic marker, the ARS/CEN replicon and the DsRed.SKL gene under the control of the Scer•MET25 promoter (Kuravi et al, 2006 J Cell Sci. 119(19):3994-4001).

Standard recombinant DNA manipulations were carried out according to Sambrook et al. (1989 Molecular cloning, a Laboratory manual. Cold Spring Harbor Laboratory, Cold Spring Harbor, N.Y.). Preparation of P. chrysogenum protoplasts and their transformation were performed in accordance with established protocols (Cantoral et al., 1987 Bio/Technol. 5, 494-497). Total DNA was isolated essentially as described by Kolar et al. (1988) Gene. 1988; 62(1):127-34. In short, protoplasts were lysed in TES/SDS buffer (10 mM Tris-HCl pH 8.0, 50 mM EDTA, 150 mM NaCl, 1% SDS) followed by phenol and chloroform extractions and ethanol precipitation. Spooled DNA was washed with 70% ethanol, air-dried, dissolved in T₁₀E₁ (10 mM Tris-HCl pH 7.4 1 mM EDTA) and treated with RNAse (10 μg/ml). A. nidulans genomic DNA was isolated according to Chow and Kafer (see http://www.fbsc.net/fgn/chow.html).

R. emersonii genomic DNA was isolated from cultures grown for 16 hours in YGG medium at 42° C., 250 rpm, using the DNeasy plant mini kit (Qiagen, Hilden, Germany).

Restriction enzymes and other DNA modifying enzymes were used in agreement with the instructions of the suppliers (Fermentas Gmbh, St. Leon-Rot, Germany; Roche Diagnostics, Mannheim, Germany)). Polymerase chain reactions (PCR) were performed with Phusion polymerase (Fermentas Gmbh) for cloning purposes and Phire polymerase (Fermentas Gmbh) for colony PCR on transformants. DNA recombination reactions were performed according to the instructions of the multisite Gateway three-fragment vector construction kit (Invitrogen, USA). Southern blot analysis was performed with Hybond N⁺ membranes (G.E. Healthcare Limited, Little Chalfont, UK), the ECL Gold hybridization buffer (GE Healthcare Limited) and DNA fragments labeled with digoxigenin using the DIG labeling and detection system (Roche Diagnostics).

P. chrysogenum, A. nidulans and A. niger DNA sequences were taken from the site of the National Center for Biotechnology Information (NCBI) (http://www.ncbi.nlm.nih.gov/), where also Blast analysis was performed. The T. reesei cbh14 sequence was taken from the website of the DOE Joint Genome Institute (http://www.jgi.doe.gov %). In silico analysis of DNA sequences and construction of vector maps was carried out using Clone Manager 5 software (Scientific and Educational Software, Durham). Alignments of amino acid sequences were constructed using Clustal_X (Thompson et al., 1997 Nucleic Acids Res 25, 4876-82) and displayed with GeneDoc (http://www.psc.edu/biomed/genedoc).

Qualitative assays of Cbh1 activity: (i) plate assay. Protoplasts of P. chysogenum DS54465 were co-transformed with the Δtif35::niaD_{F1}-amdS-niaD_{F2} deletion cassette and pDSM-JAK-120 and selected on acetamide plates. To determine which transformants had received plasmid pDSM-JAK-120, mycelia of random transformants were placed on acetamide plates containing 100 μg/ml 4-methylumbelliferyl β-D cellobioside (MUC, Sigma, Saint Louis, Mo., USA). To repress the expression of endogenous cellobiohydrolase activities the plates were also supplemented with 34.2 sucrose. After 3-5 days of growth at 25° C., the plates were visualized under UV light using a Gel Doc™ XR+Molecular Imager (Bio-Rad, Hercules, Calif., USA). Transformants that were able to convert MUC into the fluorescent substance 4-methylumbelliferone showed a clear fluorescent halo. These were also shown to harbour plasmid pDSM-JAK-120 by colony PCR.

(ii) liquid assay. Spores of P. chrysogenum Δtif35 transformants stably maintaining plasmid pDSM-JAK-120 were inoculated for 2 days at 25° C. on YGG medium supplemented with 34.2% sucrose to repress the expression of endogenous cellobiohydrolase activities. Subsequently, the cells were pelleted and aliquots of the spent medium were incubated in 50 mM sodiumacetate buffer pH 5.0, 300 μg/ml MUC for 1-16 h at 25° C. Then the reactions were stopped by the addition of 1 volume of 10% Na₂CO₃. Finally, the reaction mixtures were visualised under UV light using a Gel Doc™ XR+Molecular Imager. Media that contained significant amounts of Cbh1 activity showed clear fluorescence. As control, spent media from identically grown P. chrysogenum DS54465 cells were used, which showed no significant activity.

General Techniques

Crude extracts of Penicillium chrysogenum cells were prepared as described previously (Kiel et al., 2009). Protein concentration was determined using the RC/DC Protein Assay (Bio-Rad, USA) or the Bio-Rad Protein Assay system using bovine serum albumin as a standard. SDS-polyacrylamide gel electrophoresis and Western blotting were performed in accordance with established protocols.

Aspergillus niger transformants were selected on acetamide media and colony purified according to standard procedures, for instance as described in EP 0 635 574 B. Examples of the general design of expression vectors for gene over-expression and disruption vectors, transformation, use of markers and media can be found in WO2005/095624 and EP 0 635 574 B.

Rasamsonia emersonii transformants were selected on phleomycin media and colony purified and tested according to procedures as described in WO2011/054899.

Gene replacement vectors were designed according to known principles and constructed according to routine cloning procedures. In essence, these vectors comprise approximately 1-2 kb flanking regions of the respective ORF sequences, to target for homologous recombination at the predestined genomic loci. They may contain the A. nidulans bi-directional amdS selection marker for transformation. The method applied for gene replacements in all examples herein uses linear DNA, which integrates into the genome at the homologous locus of the flanking sequences by a double cross-over, thus substituting the gene to be deleted by the amdS gene. Loss of the amdS marker can be select for by plating on fluoro-acetamide media.

Analysis of Fluorescence in Colonies and Cells.

Colonies showing DsRed fluorescence were identified using a Night Sea Blue Star high intensity LED flashlamp and VG1 filter glasses (Tektite Industries Inc., Trenton, N.J., USA; http://wmw.nightsea.com/gfp.htm). Although this lamp is mainly used for GFP fluorescence, it is also functional for DsRed, when fluorescence is strong.

Fluorescence microscopy studies were performed using a Zeiss Axioskop microscope (Carl Zeiss, GOttingen, Germany).

### Example 1 (Comparative Example)

In this comparative example, the stability of the AMA1 plasmid in P. chrysogenum NHEJ-deficient cells was investigated.

To create an AMA1-containing control plasmid with a constitutively expressed DsRed.SKL gene, a 2108 bp DNA fragment comprising the [P_{Anid•gpdA}-DsRed.SKL-T_{Pchr•penDE}] cassette was isolated from plasmid pBBK-007 with EcoRV+NotI and cloned between the BglII (blunted by Klenow treatment) and NotI sites of plasmid pAMPF21* yielding plasmid pDSM-JAK-109.

P. chrysogenum DS54465 protoplasts were transformed with plasmid pDSM-JAK-109 (FIG. 1.), which contains a dominant phleomycin resistance marker and a constitutively produced DsRed.SKL protein (SEQ ID. NO. 35) that can be easily visualized by fluorescent techniques. DsRed.SKL ORF is shown in SEQ ID NO: 34. Upon illumination with the high intensity LED flashlamp, all Phleo⁺ transformants displayed red fluorescence. However, when mycelia was allowed to sporulate on non-selective R-agar plates, and the resulting spores plated on non-selective media, most of the resulting colonies had already lost the plasmid (>90% non-fluorescent “white” colonies). This confirms the instable nature of AMA1 plasmids in P. chrysogenum NHEJ-deficient cells.

In the following Examples we will show that replicating vectors, such as the AMA1 plasmid, which contain an essential gene as selection marker are fully stable in a host that lacks the genomic copy of this gene. One way to do this is by co-transformation during which the essential gene is deleted, while simultaneously the lethal effect of its deletion is complemented by the presence of the essential gene on the replicating vector.

### Example 2

### Preparation of Expression Construct pDSM-JAK-105 with Inducible Promoter

As putative essential gene, to be used as marker for a novel vector, we choose the P. chrysogenum tif35 gene (Pc22g19890), encoding the g subunit of the core complex of translation initiation factor 3 (eIF3g; Phan et al., 1998 Mol Cell Biol. 18(8):4935-46). Pchr•tif35 gene, ORF, cDNA and protein are shown in SEQ ID NO. 37, 38, 39 and 40, respectively Plasmid pDSM-JAK-105 allowing creation of a strain in which P. chrysogenum tif35 is placed under the control of the inducible niiA promoter was constructed using Gateway Technology as follows.

A 3951 bp DNA fragment comprising the complete P. chrysogenum niaD gene and the niiA promoter region (nt 2778005 to 2781898 in Genbank AM920428.1) was amplified with the following oligonucleotides:

DSM-JAK-101
(SEQ ID NO: 1)
5′-GGGGACAAGTTTGTACAAAAAAGCAGGCTGATCGAAGGAAGCAGTCC
CTACACTC-3′
DSM-JAK-102
(SEQ ID no. 2)
5′-GGGGACCACTTTGTACAAGAAAGCTGGGTTGAGACTGAACAATGTGA
AGACGGAG-3′

using genomic DNA of strain DS17690 as template and recombined into vector pDONR 221 yielding plasmid pDSM-JAK-101.

A 1576 bp DNA fragment comprising a region approx. 1.5 kb upstream of the P. chrysogenum tif35 coding sequence (CDS; nt 4686813 to 4688338 in Genbank AM920437.1) was amplified with the following oligonucleotides

DSM-JAK-103a
(SEQ ID NO. 3)
5′-GGGGACAACTTTGTATAGAAAAGTTGAGCATATTCTTTCACTGTTGC
AGATCTGC-3′
DSM-JAK-104a
(SEQ ID NO. 4)
5′-GGGGACTGCTTTTTTGTACAAACTTGCTATCCCATCCAGATGAGTGC
TTCG-3′

using P. chrysogenum DS54465 DNA as template and recombined into vector pDONR P4-P1R yielding plasmid pDSM-JAK-102.

A 1493 bp DNA fragment comprising the P. chrysogenum tif35 CDS and terminator region (nt 4689913 to 4691355 in Genbank AM920437.1) was amplified with the following oligonucleotides:

DSM-JAK-105
(SEQ ID NO. 5)
5′-GGGGACAGCTTTCTTGTACAAAGTGGACACCATGTCTCCAACCGGGA
AGTGAG-3′
DSM-JAK-106
(SEQ ID NO. 6)
5′-GGGGACAACTTTGTATAATAAAGTTGGGTGCTTGGGATGTTCCATGG
TAGC-3′

using P. chrysogenum DS54465 DNA as template and recombined into vector pDONR P2R-P3, yielding plasmid pDSM-JAK-103.

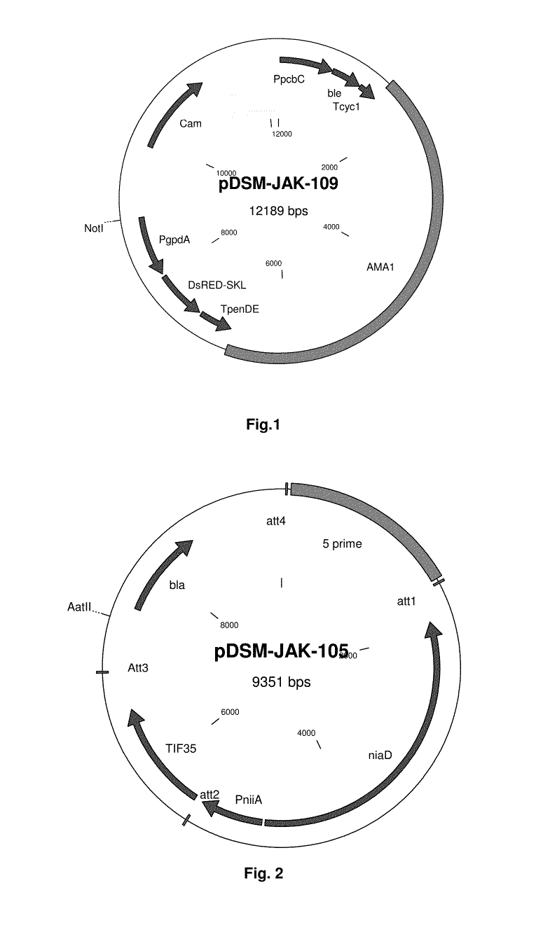

Plasmids pDSM-JAK-101, pDSM-JAK-102 and pDSM-JAK-103 were recombined with vector pDEST R4-R3, yielding pDSM-JAK-105 (FIG. 2) which allowed for the creation of a strain in which P. chrysogenum tif35 is placed under the control of the inducible niiA promoter. In this construct, the niaD gene encoding nitrate reductase functions as a selection marker.

### Example 3

### Construction of a P. Chrysogenum Strain with a Nitrate-Inducible tif35 Gene

Plasmid pDSM-JAK-105 of Example 2 was linearized with AatII in the vector region and transformed into protoplasts of P. chrysogenum DS58274. In this strain an inactivated niaD locus carries a GFP.SKL expression cassette allowing its use as both auxotropic marker as well as a transformation control. Using fluorescence microscopy, we observed that out of 52 nitrate-prototrophic transformants analysed, 44 had retained GFP fluorescence. Colony PCR using the following oligonucleotides:

| | |
|---|---|
| DSM-JAK-109 | |
| 5′-CAGTTTACACTCAACCCCAATCCAG-3′ | (SEQ ID NO. 7) |
| 3-prime-niaD-forward | |
| 5′-AGGTTGGTGGAGAAGCCATTAG-3′ | (SEQ ID NO. 8) |

showed that at least half of these carried the P_{niiA}-tif35 locus correctly recombined at the tif35 locus. Multiple independent [P_{niiA}-tif35]-containing transformants were identified, purified by sporulation and NO₃⁺ selection and further analysed. Conidiospores were produced on R agar plates supplemented with nitrate and allowed to germinate on plates containing either ammonium or nitrate as sole nitrogen source. Spore germination was fully absent on ammonium plates, but was normal on nitrate plates. This implies that Pchr•tif35 is indeed an essential gene.

### Example 4

### Construction of a P. Chrysogenum tif35 Deletion Cassette

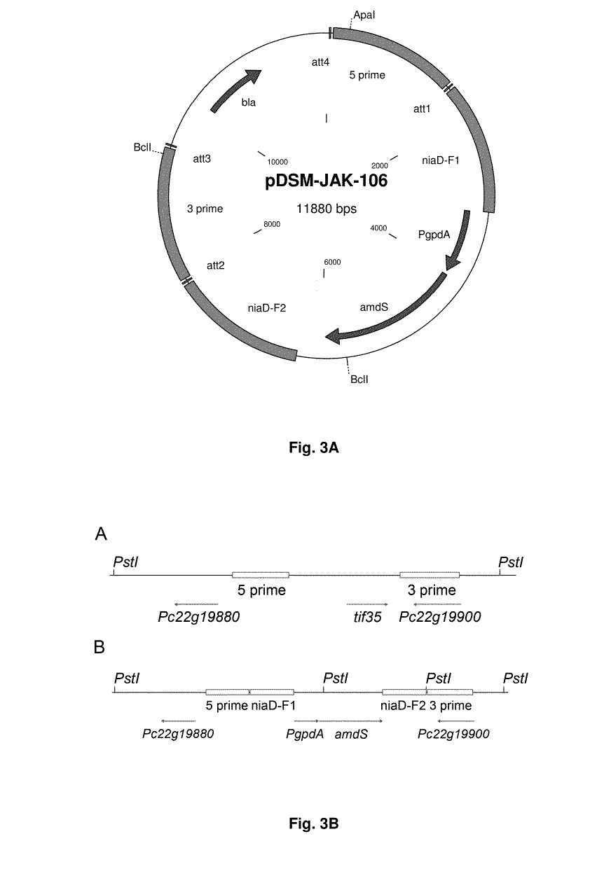

To delete the genomic copy of the P. chrysogenum tif35 gene, plasmid pDSM-JAK-106 (FIG. 3) was constructed by Gateway technology. A 1654 bp DNA fragment comprising the region downstream from the P. chrysogenum tif35 terminator (nt 4691355 to 4692956 in Genbank AM920437.1) was amplified with the following oligonucleotides:

DSM-JAK-107
(SEQ ID NO. 9)
5′-GGGGACAGCTTTCTTGTACAAAGTGGATGGGAAACTAACCACGTGCT
TGTACG-3′
DSM-JAK-108
(SEQ ID NO. 10)
5′-GGGGACAACTTTGTATAATAAAGTTGTTCACCCTGTCTCGACTTCCT
TGTC-3′

using P. chrysogenum DS54465 DNA as template, recombined into vector pDONR P2R-P3, yielding plasmid pDSM-JAK-104. Plasmids pDSM-JAK-102, pENTR221-niaD_{F1}-amdS-niaD_{F2} and pDSM-JAK-104 were recombined with vector pDEST R4-R3, yielding plasmid pDSM-JAK-106 (FIG. 3).

### Example 5

### Construction of an Alternative P. Chrysogenum tif35 Deletion Cassette

For easier separation of the Δtif35 cassette from vector DNA, a derivative of plasmid pDSM-JAK-106 from Example 4 with an extra ApaI site was constructed. For the construction of this plasmid, a 1660 bp DNA fragment comprising the region downstream from the P. chrysogenum tif35 terminator (nt 4691355 to 4692956 in Genbank AM920437.1) was amplified with the following oligonucleotides:

DSM-JAK-107
(SEQ ID NO. 9)
5′-GGGGACAGCTTTCTTGTACAAAGTGGATGGGAAACTAACCACGTGCT
TGTACG-3′
DSM-JAK-123
(SEQ ID NO. 11)
5′-GGGGACAACTTTGTATAATAAAGTTGTGGGCCCTCACCCTGTCTCGA
CTTCCTTGTC-3′

using P. chrysogenum DS54465 DNA as template, recombined into vector pDONR P2R-P3, yielding plasmid pDSM-JAK-121. Plasmids pDSM-JAK-102, pENTR221-niaD_{F1}-amdS-niaD_{F2} and pDSM-JAK-121 were recombined with vector pDEST R4-R3, yielding plasmids pDSM-JAK-122.

### Example 6

### Construction of AMA1 Plasmid pDSM-JAK-108 Containing P. Chrysogenum tif35 and DsRed.SKL Marker

An AMA1 plasmid containing P. chrysogenum tif35 was constructed as follows. A 1368 bp DNA fragment comprising the constitutive promoter of the Aspergillus nidulans AN0465 gene encoding the ribosomal protein S8 (nt 3414332 to 3415681 in Genbank BN001308) was amplified with oligonucleotides

DSM-JAK-201
(SEQ ID. NO. 12)
5′-AGAGGTACCGAGTTATAGACGGTCCGGCATAGG-3′.
DSM-JAK-202
(SEQ ID. NO. 13)
5′-AGAGGATCCGTTTGCTGTCTATGTGGGGGACTG-3′.

using genomic DNA from A. nidulans FGSC A4 (ATCC38163) as template. The PCR fragment was digested with Asp718i+BamHI and cloned between the Asp718i and BamHI sites of plasmid pBBK-001, thereby replacing the P. chrysogenum pcbC promoter. The resulting plasmid was designated pDSM-JAK-201.

An 886 bp DNA fragment comprising the terminator of the A. nidulans act (AN6542) gene encoding gamma actin (nt 2366704 to 2365833 in Genbank BN001301) was amplified with oligonucleotides

DSM-JAK-203
(SEQ ID. NO. 14)
5′-GGGGTGCTTCTAAGGTATGAGTCGCAA-3′.
DSM-JAK-204
(SEQ ID. NO. 15)
5′-AGAACGCGTTAACGCAGGGTTTGAGAACTCCGATC-3′.

using A. nidulans FGSC A4 DNA as template. The PCR fragment was digested with MluI and cloned between the SmaI and MluI sites of plasmid pDSM-JAK-201, thereby replacing the P. chrysogenum penDE terminator. The resulting plasmid was designated pDSM-JAK-202.

A 2971 bp fragment containing the [P_{AN0465}-DsRed.SKL-T_{Anid•act}] expression cassette was isolated from plasmid pDSM-JAK-202 with HpaI and KpnI and cloned into plasmid pAMPF21*, a derivative of plasmid pAMPF21 that was modified by removing specific restriction sites (HindIII, Asp718i) in the AMA1 region, digested with HindIII (blunted by Klenow treatment)+KpnI, thus yielding plasmid pDSM-JAK-107.

A 3036 bp DNA fragment comprising the tif35 coding sequence together with its promoter and terminator regions (nt 4688344 to 4691352 in Genbank AM920437.1) was amplified with oligonucleotides

DSM-JAK-111
(SEQ ID. NO. 16)
5′-AGAGGATCCGAGGAAGACGTGATCAGAGTAAGC-3′.
DSM-JAK-112
(SEQ ID. NO. 17)
5′-GAAAGCGGCCGCGGTACCGTGCTTGGGATGTTCCATGGTAGC-3′.

using genomic P. chrysogenum DS54465 DNA. The PCR fragment was digested with NotI and BamHI and cloned between the NotI and BglII sites of plasmid pDSM-JAK-107, yielding plasmid pDSM-JAK-108 (FIG. 4).

In this way an E. coli/P. chrysogenum shuttle vector was constructed which contains the Pchr•tif35 expression cassette, the AMA1 replicon for extra-chromosomal replication, and a constitutively expressed DsRed.SKL gene that can be easily visualized by fluorescent techniques. The [P_{AN0465}-DsRed.SKL-T_{Anid•act}] and Pchr•tif35 expression cassettes as present on plasmid pDSM-JAK-108 are shown in SEQ ID No. 36 and 37, respectively. Since the expression signals of the DsRED.SKL cassette on this plasmid originate from the A. nidulans genome, plasmid pDSM-JAK-108 has no significant similarity with the genome of a P. chrysogenum strain other than tif35, nor to most other filamentous fungi including Aspergillus niger.

### Example 7

### Stabilised Vector Host-System in P. Chrysogenum

Plasmid pDSM-JAK-108 was co-transformed in circular form with a P. chrysogenum Δtif35 cassette into protoplasts of P. chrysogenum DS54465 or DS61187. The Δtif35 cassette was released either from pDSM-JAK-106 (Example 3) by ApaI+ partial BclI digestion (yielding a 9119 bp DNA fragment) or from pDSM-JAK-122 (Example 4) by ApaI digestion (yielding a 9224 bp DNA fragment) and purified from agarose gel.

Transformants were selected on acetamide plates. Red fluorescent colonies harbouring the DsRed.SKL-expressing plasmid were identified with high frequency (30-50%). In the majority of the cases plasmid pDSM-JAK-108 was present in a fully intact form as demonstrated by colony PCR using oligonucleotides DSM-JAK-201 (SEQ ID. NO.12) and DSM-JAK-204 (SEQ ID. NO. 15) that amplify a 2971 bp fragment containing the [P_{AN0465}-DsRed.SKL-T_{Anid•act}] expression cassette (SEQ ID. No.36), by Southern blotting and by retransformation into E. coli DH5α followed by extensive restriction analysis. We observed that the red fluorescent phenotype was fully stable during continued mycelial growth on non-selective media and also upon conidiospore formation and germination on non-selective medium for at least ten cycles. Also curing the strain of the amdS marker by selection on replication slippage events using fluoroacetamide plates did not affect the segregational and recombinational stability of the plasmid. This implies the presence of a fully stable replicating plasmid in P. chrysogenum cells.

Deletion of the genomic copy of tif35 in transformants was demonstrated by colony PCR using the following oligonucleotide combinations:

DSM-JAK-109:
(SEQ ID. NO. 7)
5′-CAGTTTACACTCAACCCCAATCCAG-3′ +
5-prime-niaD-return:
(SEQ ID. NO. 18)
5′- CACGTAGCATACAACCGTGTCG -3′
(expected 1647 bp)
and
3-prime-niaD-forward
(SEQ ID. NO. 8)
5′- AGGTTGGTGGAGAAGCCATTAG-′3 +
DSM-JAK-110
(SEQ ID. NO. 19)
5′- GATGCCTTGTGGGAAATTAACCAG -′3.
(expected 1776 bp)

These should only amplify a DNA fragment of the indicated size upon correct recombination at the tif35 locus. Multiple independent PCR positive transformants were identified and purified by sporulation and selection of single spores on acetamide selection plates. Southern blot analysis showed correct deletion of tif35. Multiple independent Δtif35 strains carrying a replicating plasmid with the complementing tif35 gene were identified.

### Example 8

### Construction of Marker Free Strains

In the Δtif35 strains, the Anid•amdS marker is flanked by a 1.5 kb repeat comprising part of P. chrysogenum niaD (F1 and F2), allowing loss of the marker by replication slippage. To obtain marker-free strains, four independently isolated Δtif35 strains were placed on sporulation agar and streaked out to single spore on fluoroacetamide plates. From each plate two independent colonies were selected, purified by another round of sporulation and re-selection of single spores on fluoroacetamide plates. Southern blot analysis showed correct removal of the amdS marker from the tif35 locus.

### Example 9

### The Stability of AMA1 Plasmid pDSM-JAK-108 Carrying a tif35 Expression Cassette is Dependent on the Deletion of the Genomic Copy of the tif35 Gene

To demonstrate that the stability of plasmid pDSM-JAK-108 was determined by the absence of the genomic copy of the tif35 gene and not caused by recombination with the genome, an additional copy of P. chrysogenum tif35 was placed at the genomic niaD locus. To this end plasmid pDSM-JAK-116 was constructed by Gateway technology as follows:

A 3070 bp DNA fragment comprising the tif35 CDS together with its promoter and terminator regions (nt 4688343 to 4691352 in Genbank AM920437.1) was amplified with oligonucleotides

DSM-JAK-119
(SEQ ID. NO. 20)
5′- GGGGACAAGTTTGTACAAAAAAGCAGGCTGA
GAGGAAGACGTGATCAGAGTAAGC-3′
DSM-JAK-120
(SEQ ID. NO. 21)
5′- GGGGACCACTTTGTACAAGAAAGCTGGGTT
GTGCTTGGGATGTTCCATGGTAGC -3′

using P. chrysogenum DS54465 genomic DNA as template and recombined into plasmid pDONR 221, yielding plasmid pDSM-JAK-115.

Plasmids pENTR41-niaD_{L}, pDSM-JAK-115 and pENTR23-niaD_{R} were recombined with vector pDEST R4-R3 yielding plasmid pDSM-JAK-116 (FIG. 5).

The [niaD_{L}-tif35-niaD_{R}] integration cassette was released from plasmid pDSM-JAK-116 by NotI+KpnI digestion, purified from agarose gels (as a 6778 bp DNA fragment), and transformed into protoplasts of two independently isolated strains of pDSM-JAK-108 (Δtif35::niaD_{F1}-amdS-niaD_{F2}). Transformants were selected on chlorate plates, followed by colony PCR using oligonucleotides

| (SEQ ID. NO. 22) | |
|---|---|
| DSM-JAK-126 | 5′- GTTCTTGAATAGCCGAGGACTCAC -3′ |
| (SEQ ID. NO. 23) | |
| DSM-JAK-127 | ′5′- CATCCTCCCCTTCTGTTGGCATAG -3′ |

that should only amplify a DNA fragment of 1923 bp upon correct integration of the tif35 gene at the niaD locus. Multiple independent PCR-positive transformants were identified. All of these had lost the red fluorescent phenotype, indicating a loss of the replicating plasmid pDSM-JAK-108. Eight transformants were further analysed by Southern blotting and demonstrated correct integration of tif35 at the niaD locus, the presence of the original Δtif35::niaD_{F1}-amdS-niaD_{F2} locus and the absence of the replicating plasmid pDSM-JAK-108. Thus, plasmid pDSM-JAK-108 is highly stable in a strain lacking tif35, but becomes mitotically unstable again when chromosomal tif35 expression is restored, confirming its extra-chromosomal nature.

### Example 10

### Construction of an AMA1-Containing Plasmid Expressing a Trichoderma Reesei cbh1 Gene and the P. Chrysogenum tif35 Gene

To demonstrate that the newly developed stable host/vector system can be used for biotechnological purposes, we expressed the Trichoderma reesei cbh1 gene encoding a secreted cellobiohydrolase from a replicating plasmid carrying Pchr•tif35 as selection marker. An expression cassette comprising a codon pair-optimized T. reesei cbh1 gene flanked by non-homologous A. nidulans expression signals was constructed as follows.

A 1157 bp DNA fragment comprising the constitutive promoter of the A. nidulans tef gene (AN4218) encoding the translation elongation factor alpha (nt 1654144 to 1655266 in Genbank BN001302.1) was amplified with oligonucleotides

DSM-JAK-205
(SEQ ID. NO. 24)
5′-AGAAAGCTTGGTACCGTTGCACCAATCGCCGTTTAGG -3′
DSM-JAK-206
(SEQ ID. NO. 25)
5′- AGAAGATCTGTCGACGAATTCGGTGAAGGTTGTGTTATG
TTTTGTGG -3′

using A. nidulans FGSC A4 genomic DNA as template. The PCR fragment was digested with HindIII+BglII and cloned between the HindIII and BglII sites of plasmid pENTR221-stuffer, yielding plasmid pDSM-JAK-203.

A 705 bp DNA fragment comprising the terminator of the A. nidulans trpC gene (AN0648) encoding anthranilate synthase component 2 (nt 2848474 to 2849165 in Genbank BN001308.1) was amplified with oligonucleotides

DSM-JAK-210
(SEQ ID. NO. 26)
5′-AGAAGATCTGATCGTTGGTGTCGATGTCAGCTC-3′
DSM-JAK-211
(SEQ ID. NO. 27)
5′- GGGGTACACAGTACACGAGGACTTCTAG -3′

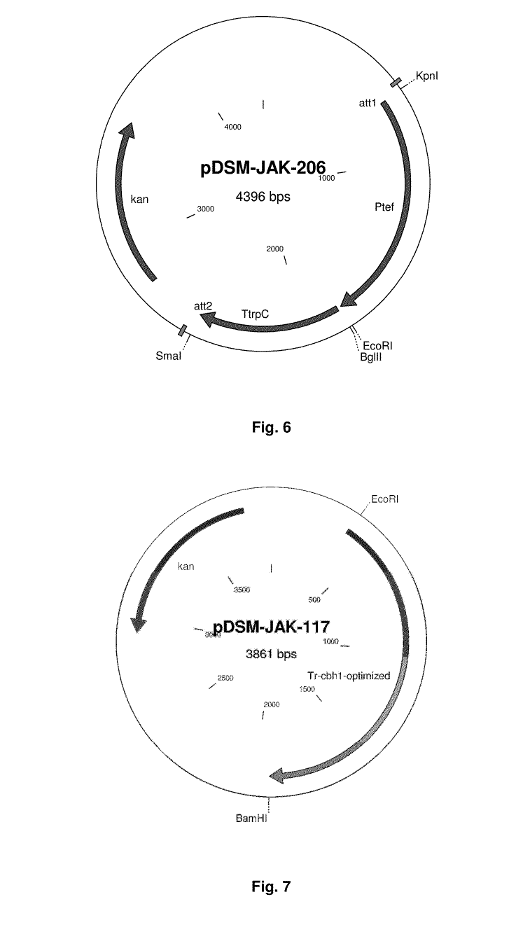

using A. nidulans FGSC A4 DNA as template. The PCR fragment was digested with BglII and cloned between the BglII and SmaI sites of plasmid pDSM-JAK-203, yielding plasmid pDSM-JAK-206 (FIG. 6).

The wild type T. reesei cbh1 cDNA (SEQ ID. No. 28) was obtained from the website of the DOE Joint Genome Institute and optimized for expression in P. chrysogenum and A. niger.

A 1583 bp DNA fragment comprising the codon pair optimized Tree•cbh1 cDNA sequence was synthesized at GeneArt AG (Regensburg, Germany), digested with Sfi1 and cloned into Sfi1-linearized vector pMK-RQ (Gene Art). The resulting plasmid was designated pDSM-JAK-117 (FIG. 7).

A 1564 bp DNA fragment comprising the codon pair optimized Tree•cbh1 cDNA sequence was isolated from pDSM-JAK-117 with EcoRI+BamHI and cloned between the EcoRI and BglII sites of plasmid pDSM-JAK-206, yielding pDSM-JAK-118.

A 3387 bp DNA fragment comprising the P_{Anid•tef}_{^{−}}Tree-cbh1^{opt}-T_{Anid•trpC} expression cassette was isolated from pDSM-JAK-118 with KpnI+SmaI and cloned between the KpnI and HindIII (blunted by Klenow treatment) sites of plasmid pAMPF21*, yielding plasmid pDSM-JAK-119.

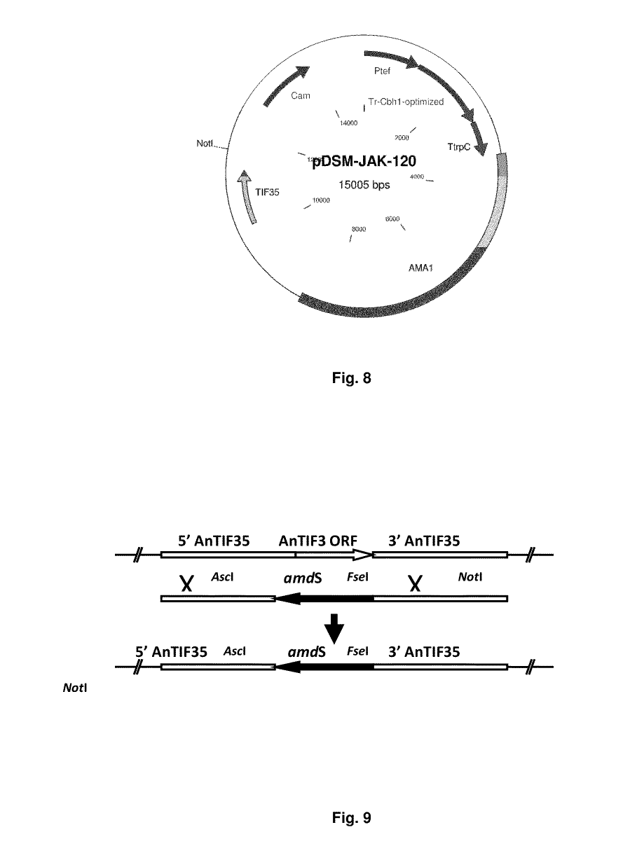

A 3036 bp DNA fragment comprising the tif35 coding sequence together with its promoter and terminator regions (nt 4688344 to 4691352 in Genbank AM920437.1) was amplified with oligonucleotides DSM-JAK-111 (SEQ ID. NO. 16) and DSM-JAK-112 (SEQ ID. NO. 17) using genomic P. chrysogenum DS54465 DNA. The PCR fragment was digested with NotI and BamHI and cloned between the NotI and BglII sites of plasmid pDSM-JAK-119, yielding plasmid pDSM-JAK-120 (FIG. 8).

### Example 11

### Production of a Heterologous Protein from a Stabilised AMA1-Plasmid in P. chrysogenum

Plasmid pDSM-JAK-120 was co-transformed in circular form with the P. chrysogenum Δtif35::niaD_{F1}-amdS-niaD_{F2} cassette from pDSM-JAK-122 (Example 5) into protoplasts of P. chrysogenum DS54465. Transformants were selected on acetamide plates. Multiple independent transformants harbouring the Tree-cbh1^{opt} expressing plasmid were identified with high frequency (30-40%) by colony PCR using oligonucleotides DSM-JAK-205 (SEQ ID. NO.24) and DSM-JAK-211 (SEQ ID. NO.27) that amplify the [P_{Anid•tef}-Tree•cbh1^{opt}-T_{Anid•trpC}] cassette. Furthermore, these colonies were analysed for the production of active Tree-cbh1^{opt} enzyme on acetamide selection plates supplemented with 4-methylumbelliferyl β-D-cellobioside (MUC). The plasmid-containing transformants indeed secreted cellobiohydrolase as determined by Mass Spec and activity measurements. The stability of transformants was demonstrated by at least four rounds of successive sporulation/germination on non-selective R-agar plates followed by colony formation from single spores on acetamide plates, a procedure that does not select for the plasmid.

### Example 12

### AMA1 Plasmid Stabilisation in A. niger

The same AMA1 plasmid which was used in the previous Examples for the transformation of Penicillium, was subsequently used to test the system of the present invention in the biotechnologically important filamentous fungus A. niger. First, the A. niger tif35 gene was identified by alignment (An16g05260) Anig•tif35 gene, ORF, cDNA and protein are shown in SEQ ID NO. 29, 30, 31 and 32, respectively. An Anig•tif35::Anid•amdS deletion cassette (SEQ ID No. 33) was constructed by cloning a functional amdS cassette, comprising the A. nidulans amdS ORF and terminator, expressed from the A. nidulans gpdA promoter, between 5′ and 3′ flanking regions of Anig•tif35. To this end, the flanking regions were PCR amplified from genomic DNA of WT2 using primers comprising unique restriction sites (Not1, AscI and FseI, as indicated in FIG. 9) to facilitate cloning into a suitable E. coli vector Then A. niger strain WT 2 was transformed according to routine procedures with the AMA1 plasmid pDSM-JAK-108 (FIG. 4), containing the DsRed.SKL marker and the Pchr•tif35 homologue and a linear NotI fragment containing the Anig•tif35::Anid•amdS deletion cassette. Correct integration by double homologous recombination would result in substitution of the Anig•tif350RF (including part of its promoter) by amdS. Approximately 350 transformants were obtained by selection on acetamide medium and about 90% were red when irradiated with blue light. Colonies of the non-transformed WT 2 did not show the red colour but had the normal “white” colour. A subset of 40 red transformants was inspected in more detail.

These transformants were colony purified by cultivation on non-selective (PDA) medium (Potato Dextrose Agar, Oxoid) after which spores were re-streaked on PDA plates to obtain single colonies. From each transformants, virtually all single colony isolates remained red. The red colour persisted even after five subsequent cycles of sporulation and single colony isolation on PDA medium as described above. This showed that the DsRed.SKL marker was stably present even after prolonged cultivation on non-selective medium. In our case we tested for 10 subsequent cycles of sporulation and single colony isolation. PCR diagnostics confirmed that in all 40 transformants tested, the genomic Anig•tif35 was substituted by the amdS cassette. Southern blot and transformation of E. coli with total DNA from these same transformants confirmed the presence of the intact episomal pDSM-JAK-108. This showed that the plasmid pDSM-JAK-108 was stably present even after prolonged cultivation on non-selective medium. These data imply that plasmid pDSM-JAK-108 is a suitable basis for the construction of multi-purpose vectors that can be stably maintained in multiple filamentous ascomycetes.

### Example 13

### Identification of the ReKu80 Gene of Rasamsonia emersonii and Construction of Deletion Vectors

Genomic DNA of Rasamsonia emersonii strain CBS393.64 was sequenced and analyzed. The gene with translated protein annotated as homologues to known ReKu80 gene was identified.

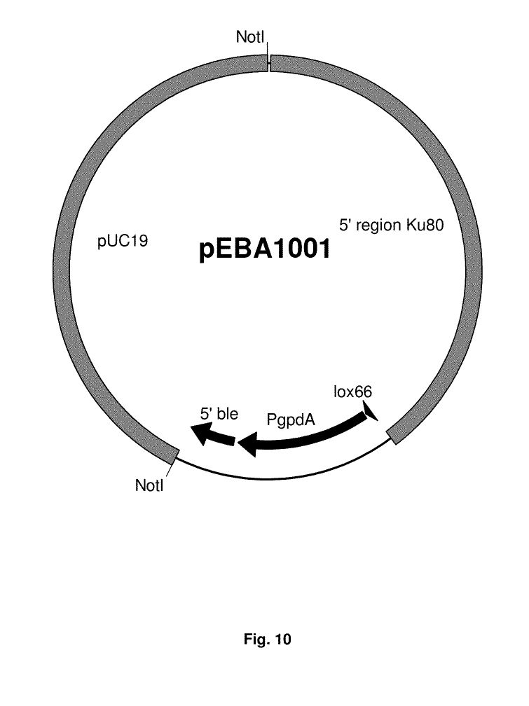

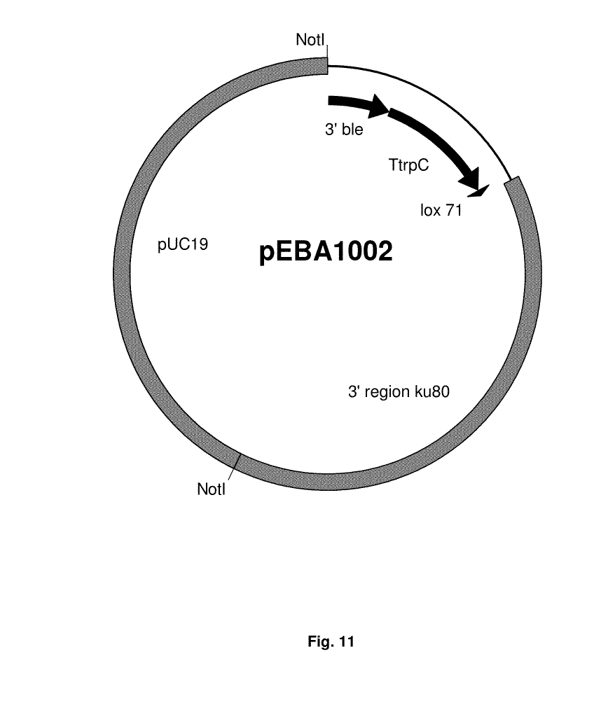

Sequences of the R. emersonii ReKu80 gene, comprising the genomic sequences of the open reading frames (ORF) (with introns) and approximately 2500 bp of the 5′ and 3′ flanking regions, cDNA and protein sequences, are shown in SEQ ID NOs: 41 to 43 respectively. Two replacement vectors for ReKu80, pEBA1001 and pEBA1002, were constructed according to routine cloning procedures (see FIGS. 10 and 11). The insert fragments of both vectors together can be applied in the so-called “bipartite gene-targeting” method (Nielsen et al., 2006, 43: 54-64). This method is using two non-functional DNA fragments of a selection marker which are overlapping (see also WO2008113847 for further details of the bipartite method) together with gene-targeting sequences. Upon correct homologous recombination the selection marker becomes functional by integration at a homologous target locus. The deletion vectors pEBA1001 and pEBA1002 were designed as described in WO 2008113847, to be able to provide the two overlapping DNA molecules for bipartite gene-targeting.

The pEBA1001 vector comprises a 2500 bp 5′ flanking region of the ReKu80 ORF for targeting in the ReKu80 locus, a lox66 site, and the 5′ part of the ble coding region driven by the A. nidulans gpdA promoter (FIG. 10). The pEBA1002 vector comprises the 3′ part of the ble coding region, the A. nidulans trpC terminator, a lox71 site, and a 2500 bp 3′ flanking region of the ReKu80 ORF for targeting in the ReKu80 locus (FIG. 11).

### Example 14

### Cloning of pEBA513 for Transient Expression of Cre Recombinase

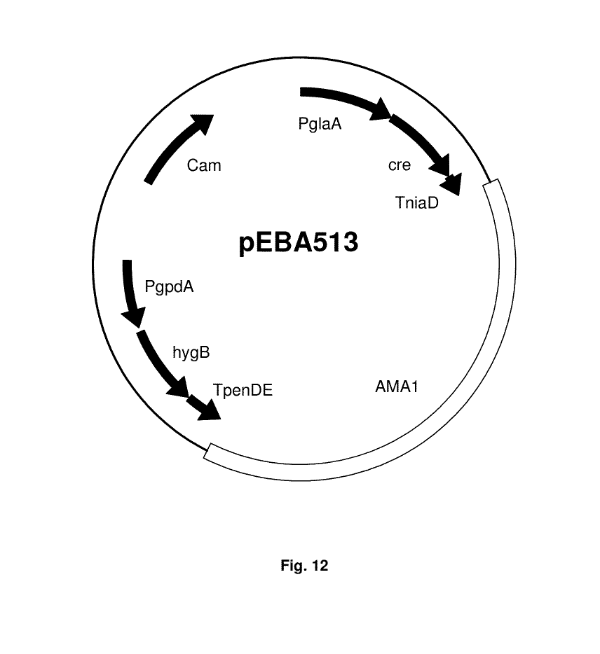

pEBA513 was constructed by DNA2.0 (Menlo Park, USA) and contains the following components: expression cassette consisting of the A. niger glaA promoter, ORF encoding cre-recombinase (AAY56380) and A. nidulans niaD terminator; expression cassette consisting of the A. nidulans gpdA promoter, ORF encoding hygromycin B resistance protein and P. chrysogenum penDE terminator (Genbank: M31454.1, nucleotides 1750-2219); pAMPF21 derived vector containing the AMA1 region and the CAT chloramphenicol resistance gene. FIG. 12 represents a map of pEBA513.

### Example 15

### Inactivation of the ReKu80 Gene in Rasamsonia Emersonii

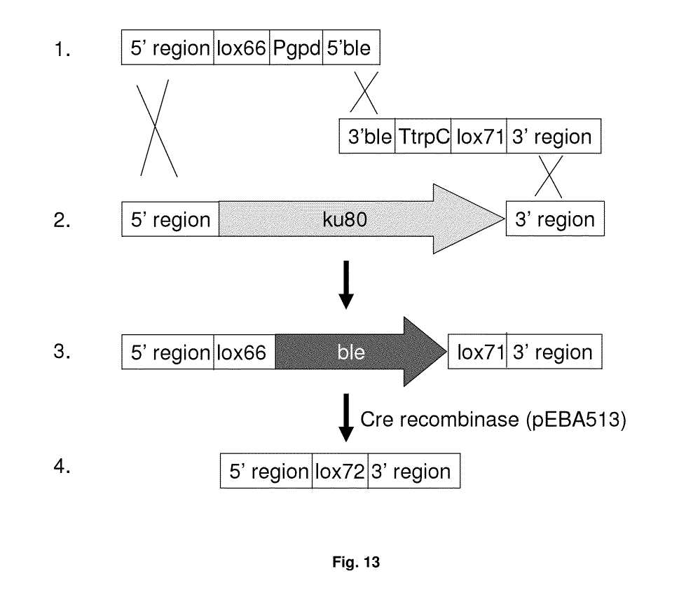

Linear DNA of the deletion constructs pEBA1001 and pEBA1002 were isolated and used to transform Rasamsonia emersonii CBS393.64 using method as described earlier in WO2011\054899. These linear DNAs can integrate into the genome at the ReKu80 locus, thus substituting the ReKu80 gene by the ble gene as depicted in FIG. 13. Transformants were selected on phleomycin media and colony purified and tested according to procedures as described in WO2011/054899. Growing colonies were diagnosed by PCR for integration at the ReKu80 locus using a primer in the gpdA promoter of the deletion cassette and a primer directed against the genomic sequence directly upstream of the 5′ targeting region. From a pool of approximately 250 transformants, 4 strains showed a removal of the genomic ReKu80 gene.

Subsequently, 3 candidate ReKu80 knock out strains were transformed with pEBA513 to remove the ble selection marker by transient expression of the cre recombinase. pEBA513 transformants were plated in overlay on regeneration medium containing 50 μg/ml of hygromycin B. Hygromycin-resistant transformants were grown on PDA containing 50 μg/ml of hygromycin B to allow expression of the cre recombinase. Single colonies were plated on non-selective Rasamsonia agar medium to obtain purified spore batches. Removal of the ble marker was tested phenotypically by growing the transformants on media with and without 10 μg/ml of phleomycin. The majority (>90%) of the transformants after transformation with pEBA513 (with the cre recombinase) were phleomycin sensitive, indicating removal of the pEBA1001 and pEBA1002-based ble marker. Removal of the pEBA513 construct in ble-negative strains was subsequently diagnosed phenotypically by growing the transformants on media with and without 50 μg/ml of hygromycin. Approximately 50% of the transformants lost hygromycin resistance due to spontaneously loss of the pEBA513 plasmid. Deletion of the ReKu80 gene and the absence of the pEBA513 plasmid was confirmed by PCR analysis and Soutern blotting

Strain deltaKu80-2 was selected as a representative strain with the Ku80 gene inactivated.

### Example 16

### Construction of a R. emersonii ReTif35 Deletion Cassette

Genomic DNA of Rasamsonia emersonii strain CBS393.64 was sequenced and analyzed. The gene with translated protein annotated as ReTif35 was identified. Sequences of the R. emersonii ReTif35 gene, comprising the genomic sequence of the ORF and approximately 1500 bp of the 5′ and 3′ flanking regions, cDNA and protein sequence, are shown in sequence listings 44 to 46.

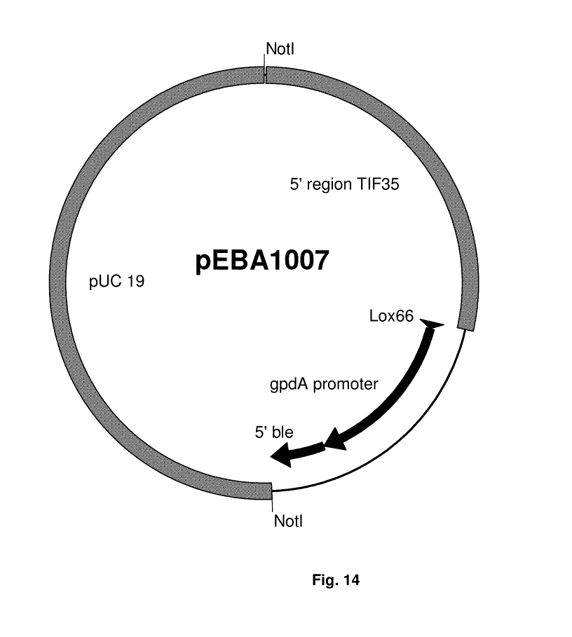

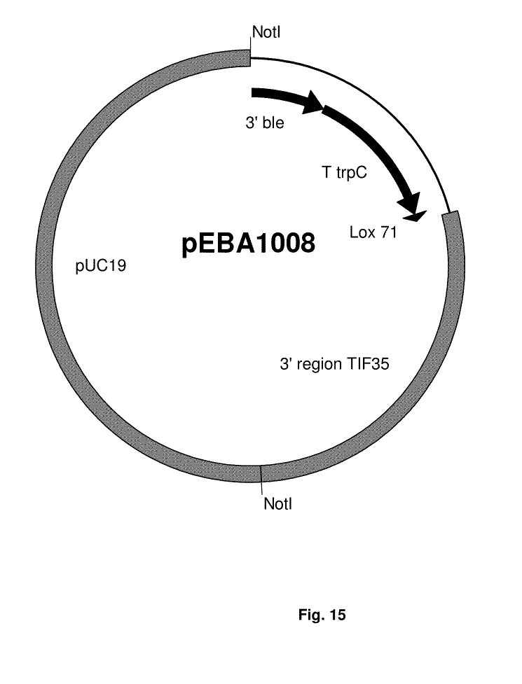

Gene replacement vectors for R. emersoniic ReTif35 gene were designed using the bipartite gene-targeting method and constructed according to routine cloning procedures (see FIGS. 14 and 15). The pEBA1007 construct comprises a 1500 bp 5′ flanking region of the ReTif35 ORF for targeting in the ReTif35 locus, a lox66 site, and the 5′ part of the ble coding region driven by the A. nidulans gpdA promoter (FIG. 14). The pEBA1008 construct comprises the 3′ part of the ble coding region, the A. nidulans trpC terminator, a lox71 site, and a 1500 bp 3′ downstream flanking region of the ReTif35 ORF for targeting in the ReTif35 locus (FIG. 15).

### Example 17

### Stabilised Vector Host-System in R. emersonii

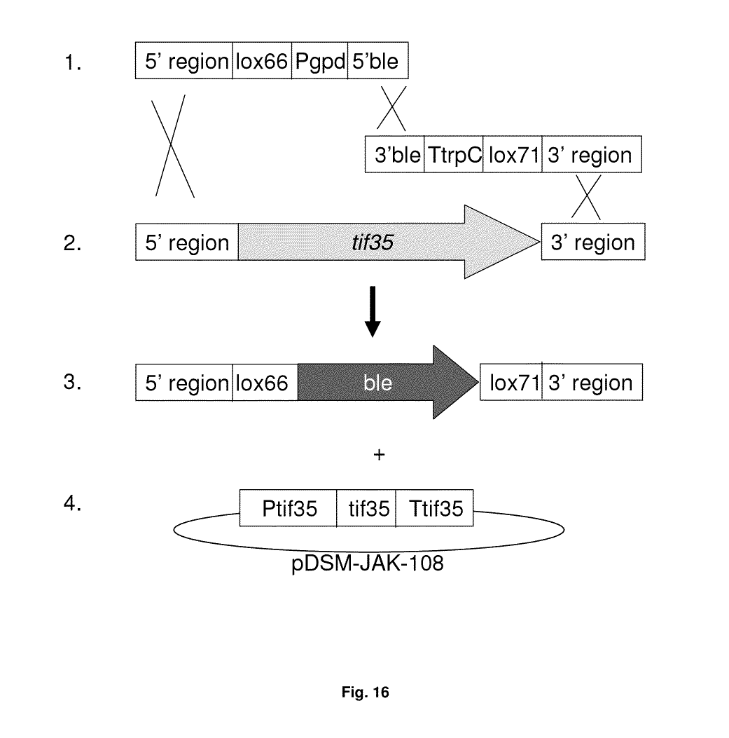

Rasamsonia emersonii CBS393.64 strain was co-transformed with circular plasmid pDSM-JAK-108 and linear DNA of the ReTif35 deletion constructs pEBA1007 and pEBA1008 using method earlier described in patent WO2011054899. The ReTif35 deletion constructs integrate at the ReTif35 locus, thus substituting the ReTif35 gene by the ble gene as depicted in FIG. 16. To compensate for the deletion of essential gene ReTif35, pDSM-JAK-108 was co-transformed carrying the P. chrysogenum tif35 expression expression cassette. In order to easily detect the presence of the pDSM-JAK-108 plasmid, the plasmid also contains a DsRed.SKL expression cassette. Transformants were selected on phleomycin media as described in WO2011054899.

Red fluorescent colonies harbouring the tif35- and DsRed.SKL-expressing plasmid were identified with high frequency (˜30%). Red transformants were colony purified by cultivation on non-selective Rasamsonia agar medium after which spores were re-streaked on Rasamsonia agar medium plates to obtain single colonies. From each transformant, all single colony isolates remained red. The red colour persisted even after nine subsequent cycles of sporulation and single colony isolation on Rasamsonia agar medium as described above. This showed that the DsRed.SKL marker was stably present even after prolonged cultivation on non-selective medium.

In contrast, in transformants that were co-transformed with pDSM-JAK-108 and pAN8 that still contained the essential gene ReTif35 in its genome, some red colonies were observed 4 days after transformation, but after 7 days no red colonies were observed at all. These data demonstrate that without selection using the essential gene tif35 the AMA1 plasmid is rapidly lost during propagation in Rasamsonia emersonii.

PCR diagnostics and Southern blots confirmed that in all tested red fluorescent transformants, the genomic R. emersonii ReTif35 gene was substituted by the ble cassette. In order to determine whether pDSM-JAK-108 was still episomal in the cells, Southern blots were performed using pDSM-JAK-108 plasmid as probe. To determine whether pDSM-JAK-108 was intact in the cells, total DNA was isolated from red fluorescent transformants to transform E. coli and E. coli colonies were analysed for the presence of intact pDSM-JAK-108. Both Southern blot analysis and restriction enzyme analysis of DNA isolated from E. coli transformants confirmed the presence of the intact episomal pDSM-JAK-108 in red fluorescent R. emersonii transformants. This showed that the plasmid pDSM-JAK-108 was stably present even after prolonged cultivation on non-selective medium.

In conclusion, these findings indicate that in R. emersonii strains in which the essential gene ReTif35 is deleted, the episomal AMA1 plasmid pDSM-JAK-108 expressing the Pchr•tif35 gene is mitotically stable.

### Example 18

### Construction of a P. Chrysogenum aur1 Deletion Cassette

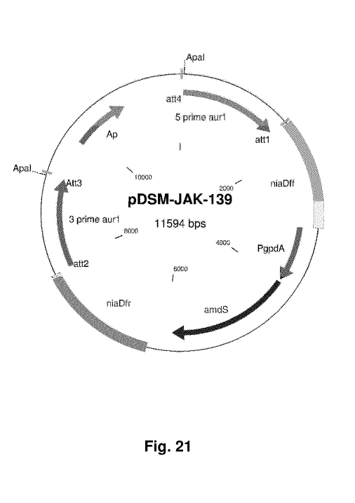

To demonstrate that the use of an essential gene as a tool to stabilize replicating plasmids in filamentous fungi is not limited to the tif35 gene, we utilized an alternative essential gene to stabilize an AMA1 plasmid in P. chrysogenum. For this we chose to use the A. nidulans aur1 gene encoding the enzyme phosphatidylinositol:ceramide phosphoinositol transferase, which is required for sphingolipid synthesis. In multiple fungal species this gene has been demonstrated to be essential. To delete the genomic copy of the P. chrysogenum aur1 (Pc12g15520) gene, plasmid pDSM-JAK-139 (FIG. 21) was constructed by Gateway technology. Two DNA fragments of 1538 bp and 1406 bp comprising the region upstream and downstream from the P. chrysogenum aur1 gene, respectively, (nt 3709510 to 3710991 and 3712509 to 3713859 in Genbank AM920427.1) were amplified with the following oligonucleotide combinations:

DSM-JAK-164
(SEQ ID NO: 47)
5′-
GGGGACAACTTTGTATAGAAAAGTTGGGCCCAACGCATGTGTACGAGAGT
CAAGG-3′ +
DSM-JAK-165
(SEQ ID NO: 48)
5′- GGGGACTGCTTTTTTGTACAAACTTGAGACGGAAGGAGATCGCGTA
ACAG -3′
and
DSM-JAK-166
(SEQ ID NO: 49)
5′- GGGGACAGCTTTCTTGTACAAAGTGGGGCGCAGTCCATTCTTGCAT
CTAC -3′ +
DSM-JAK-167
(SEQ ID NO: 50)
5′- GGGGACAACTTTGTATAATAAAGTTGGGCCCAGCCACTTCTTGTAT
CACGGAT -3′,

respectively,
using P. chrysogenum DS54465 DNA as template, recombined into vector pDONR P4-P1R and pDONR P2R-P3, respectively, yielding plasmids pDSM-JAK-137 and pDSM-JAK-138. Plasmids pDSM-JAK-137, pENTR221-niaD_{F1}-amdS-niaD_{F2} and pDSM-JAK-138 were recombined with vector pDEST R4-R3, yielding plasmid pDSM-JAK-139 (FIG. 21).

### Example 19

### Construction of AMA1 Plasmid pDSM-JAK-136 Containing Aspergillus nidulans aur1 and DsRed.SKL Marker

An AMA1 plasmid containing A. nidulans aur1 was constructed as follows. A 3438 bp DNA fragment comprising the aur1 coding sequence together with its promoter and terminator regions (nt 351475 to 348062 in Genbank BN001303.1) was amplified with oligonucleotides

DSM-JAK-162
(SEQ ID. NO: 51)
5′- AGAGAGGATCCGAGTTGGCCAGTTGACAACCTGAG -3′
and
DSM-JAK-163
(SEQ ID NO. 52)
5′- AGAGAGCGGCCGCGAGTATGAGCGATCGACACGAATG -3′,

using genomic A. nidulans FGSC A4 DNA as template. The PCR fragment was digested with NotI and BamHI and cloned between the NotI and BglII sites of plasmid pDSM-JAK-107, yielding plasmid pDSM-JAK-136 (FIG. 20). In this way an E. coli/P. chrysogenum shuttle vector was constructed which contains the Anid•aur1 expression cassette, the AMA1 replicon and the DsRed.SKL gene. Plasmid pDSM-JAK-136 has no significant similarity with the genome of a P. chrysogenum strain other than the aur1 coding sequence, nor to most other filamentous fungi including Aspergillus niger.

### Example 20

### Stabilised Vector Host-System in P. Chrysogenum Using aur1

Plasmid pDSM-JAK-136 (Example 20) was co-transformed in circular form with a P. chrysogenum Δaur1 cassette into protoplasts of P. chrysogenum DS54465. The P. chrysogenum Δaur1 cassette was released by ApaI digestion from pDSM-JAK-139 (Example 21), yielding a 9145 bp fragment, and purified from agarose gel.

Transformants were selected on acetamide plates. As described above (Example 7), red fluorescent colonies harbouring the DsRed.SKL-expressing plasmid were identified with high frequency. In the majority of the cases plasmid pDSM-JAK-136 was present in a fully intact form as demonstrated by colony PCR using oligonucleotides DSM-JAK-162 (SEQ ID. NO: 51) and DSM-JAK-163 (SEQ ID. NO: 52) that amplify a 3438 bp fragment containing the Anid•aur1 expression cassette (SEQ ID. NO: 53), by Southern blotting and by retransformation into E. coli DH5α followed by extensive restriction analysis. We observed that the red fluorescent phenotype was fully stable during continued mycelial growth on non-selective media and also upon conidiospore formation and germination on non-selective medium for at least two cycles. This again implies the presence of a fully stable replicating plasmid in P. chrysogenum cells.

Deletion of the genomic copy of aur1 in transformants was demonstrated by colony PCR using the following oligonucleotide combinations:

DSM-JAK-168
(SEQ ID. NO: 54)
5′- AGCTTTGACGCTAGATTGGAGATG -3′ +
5-prime-niaD-return (SEQ ID. NO. 18)
(expected 1647 bp)
and
DSM-JAK-169
(SEQ ID. NO: 55)
5′- CAAGCAAGCCATCTCAACAAGTGC -3′ +
3-prime-niaD-forward (SEQ ID. NO. 8)
(expected 1531 bp)

These should only amplify a DNA fragment of the indicated size upon correct recombination at the aur1 locus. Multiple independent PCR positive transformants were identified and purified by sporulation and selection of single spores on acetamide selection plates. Southern blot analysis showed correct deletion of aur1. Multiple independent Δaur1 strains carrying a replicating plasmid with the complementing Anid•aur1 expression cassette were identified.

### Example 21

### Construction of a Saccharomyces cerevisiae TIF35 Deletion Cassette

To demonstrate that the use of the AMA1 replicon in the stable replicating plasmid is not essential for the method described here, we utilized an alternative replicon to stabilize plasmids in fungi. Since no other replicons are available for filamentous fungi besides AMA1, we chose to utilize a low-copy CEN/ARS containing plasmid with baker's yeast Saccharomyces cerevisiae as a host. The S. cerevisiae TIF35 gene was chosen as essential gene to stabilize the replicating plasmid.

To delete the genomic copy of the S. cerevisiae TIF35 (YDR429C) gene, a Δtif35::loxP-KanMX4-loxP deletion cassette of 1715 bp was prepared by PCR with the oligonucleotides

DSM-JAK-139
(SEQ ID NO: 56)
5′-
TACTCGCTGTATTGAAAGGATCAAAAGACCAAAGACCACCAGGAATAATG
CCAGCTGAAGCTTCGTACGC -3′
and
DSM-JAK-140
(SEQ ID NO: 57)
5′-
ATGAGAAGAGTAACATTAGAAAACAAGTGCAGAGCATATTCTGTGCATCT
AGCATAGGCCACTAGTGGATCTG -3′

using plasmid pUG6 as template. The sequence of the deletion cassette is given in SEQ ID. NO: 58. Before use the PCR fragment was purified from agarose gel.

### Example 22

### Construction of CEN/ARS Plasmids pDSM-JAK-134 and pDSM-JAK-135 Containing Saccharomyces cerevisiae TIF35 and DsRed.SKL Marker

A CEN/ARS plasmid containing S. cerevisiae TIF35 was constructed as follows. First, we provided plasmid pUG34-DsRed.SKL with a constitutively expressed DsRed.SKL gene by replacing the S. cerevisiae MET25 promoter by the S. cerevisiae TDH3 (YGR192c) promoter. A 728 bp DNA fragment comprising the Scer•TDH3 promoter region (nt 884500 to 883790 in Genbank BK006941.2) was amplified with oligonucleotides

DSM-JAK-148
(SEQ ID. NO: 59)
5′- GAATAAAAAAGAGCTCACGCTTTTTCAGTTCGAGTTTATC -3′
and
DSM-JAK-149
(SEQ ID. NO: 60)
5′- GTTAATAGCAACTCTAACCATGGTTTGTTTGTTTATGTGTG -3′,

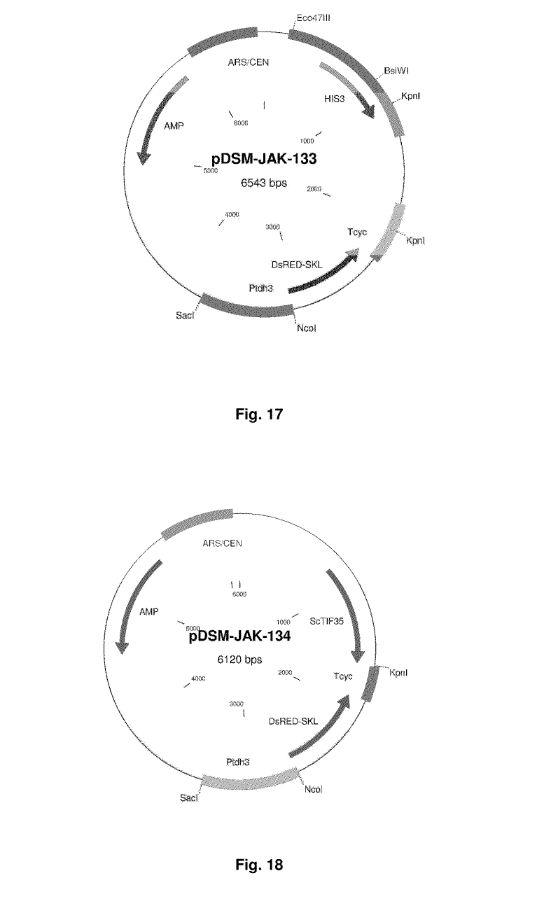

using genomic S. cerevisiae BY4742 DNA as template. The PCR fragment was digested with SacI and NcoI and cloned between the SacI and NcoI sites of plasmid pUG34-DsRed.SKL, yielding plasmid pDSM-JAK-133 (FIG. 17). In this way an E. coli/S. cerevisiae shuttle vector was constructed which contains the Scer•HIS3 auxotrophic marker, the ARS/CEN replicon and the DsRed.SKL gene. This plasmid is used as the control plasmid during stability experiments (Example 23). Subsequently, the Scer•HIS3 marker of pDSM-JAK-133 was replaced by the S. cerevisiae TIF35 gene. A 1449 bp DNA fragment comprising the Scer•TIF35 gene and its promoter region (nt 1325908 to 1324469 in Genbank BK006938.2) was amplified with oligonucleotides

DSM-JAK-137
(SEQ ID. NO: 61)
5′- GCTTATGGTGGTGGTGCTTCTTATAG -3′
and
DSM-JAK-138
(SEQ ID. NO: 62)
5′- AGAGGGTACCTGTGCATCTATTCCTTAACCTTAGG -3′,

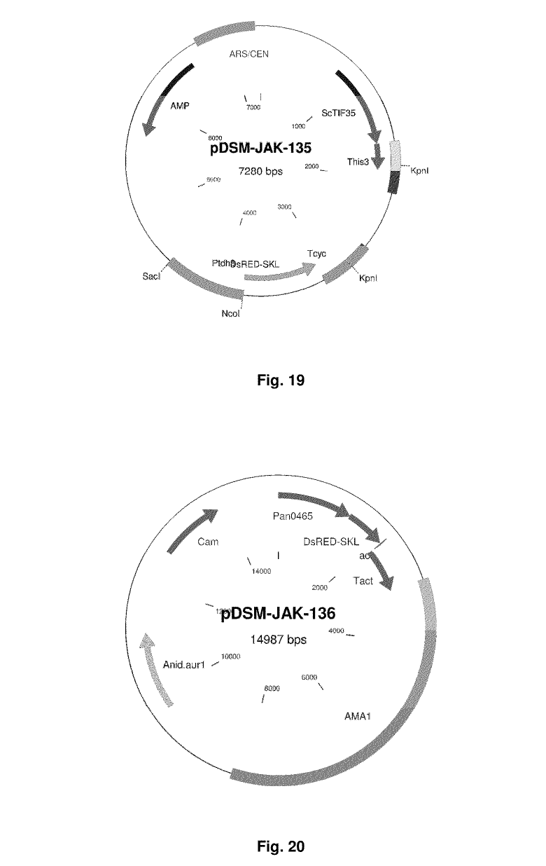

using genomic S. cerevisiae BY4742 DNA as template. The PCR fragment was digested with either KpnI or Acc651 and cloned between the Eco47111+KpnI or Eco47111+BslWI sites of plasmid pDSM-JAK-133, respectively, yielding plasmids pDSM-JAK-134 (FIG. 18) and pDSM-JAK-135 (FIG. 19).

### Example 23

### Stabilization of CEN/ARS Plasmids in the Yeast Saccharomyces cerevisiae

Plasmids pDSM-JAK-134 and pDSM-JAK-135 (Example 22) were co-transformed in circular form with the Scer•tif35 deletion cassette (Example 21) into competent cells of S. cerevisiae BY4742. Transformants were selected on geneticin (G418) plates. Using a led lamp red fluorescent colonies harbouring the DsRed.SKL-expressing plasmid were identified with high frequency (>50%). In the majority of the cases plasmid pDSM-JAK-134 or pDSM-JAK-135 was present in a fully intact form as demonstrated by retransformation into E. coli DH5α followed by extensive restriction analysis. As a control, we transformed plasmid pDSM-JAK-133 into competent cells of S. cerevisiae BY4742 with selection on histidine prototrophy. Using Fluorescence Assisted Cell Sorting (FACS), we observed that the red fluorescent phenotype in cells with either pDSM-JAK-134 or pDSM-JAK-135 was fully stable during continued growth on non-selective media for at least 38 generations. This implies the presence of a fully stable replicating plasmid in S. cerevisiae cells. In contrast, S. cerevisiae cells containing pDSM-JAK-133 lost the red fluorescence (approx. 1% per generation) when grown in medium supplemented with histidine.

Deletion of the genomic copy of TIF35 in transformants was demonstrated by colony PCR using the following oligonucleotides:

DSM-JAK-146
(SEQ ID. NO: 63)
5′- AGTACGGTCATTGGACCTGGAATC -3′
and
DSM-JAK-147
(SEQ ID. NO: 64)
5′- CGTTCCATGCACCTCCATGAATGT -′3

These should either amplify a DNA fragment of 2249 bp upon correct recombination at the TIF35 locus or one of 1454 bp when the wild type locus is present. Multiple independent PCR positive transformants were identified.

### Example 24

### Determination of the Copy Number of Stable Plasmids in P. chrysogenum by Quantitative PCR Analysis

To analyse the copy number of the stable replicating plasmids in P. chrysogenum co-transformants, we performed quantitative PCR (qPCR) using total genomic DNA. The gene copy numbers were determined with a Miniopticon™ system (Bio Rad) using the Bio Rad CFX manager software in which the C(t) values were determined automatically by regression. The SensiMix™ SYBRmix (Bioline, Alphen aan den Rijn, The Netherlands) was used as a master mix for qPCR with 0.4 μM primers and 100 ng total DNA in a 25 μl reaction volume. Copy numbers were calculated from duplicate experiments.

Copy number determination of the stable replicating plasmids pDSM-JAK-108 and pDSM-JAK-120 carrying the essential tif35 gene, in P. chrysogenum co-transformants (Examples 7 and 11, respectively) were performed using the primers

| | |
|---|---|
| DSM-JAK-174 | |
| 5′- CCACCGTTGTCCGCGAACAT -3′ | (SEQ ID NO: 65) |
| and | |
| DSM-JAK-175 | |
| 5′- TCCTTCTCGGCCTCCTTAGC -3′, | (SEQ ID NO: 66) |

which amplify a 178 bp DNA fragment of P. chrysogenum tif35. As reference, two primers,

| | |
|---|---|
| Act-F3 | |
| 5′- CTGGCGGTATCCACGTCACC -3′ | (SEQ ID NO: 67) |
| and | |
| Act-R3 | |
| 5′- AGGCCAGAATGGATCCACCG -3′ | (SEQ ID NO: 68) |

were used that amplify a 300 bp fragment of the gene encoding P. chrysogenum γ-actin (Genbank accession number AF056975) that is present in one copy in the genome. The untransformed strains DS54465 and DS61187 were used as controls, carrying each a single copy of tif35 in their genomes.

In two independent P. chrysogenum DS54465 Δtif35::niaD-amdS-niaD [pDSM-JAK-108] strains (Example 7), plasmid pDSM-JAK-108 was present in approximately 8 copies per genome, while in P. chrysogenum DS61187 Δtif35::niaD-amdS-niaD [pDSM-JAK-108] (Example 7) and P. chrysogenum DS54465 Δtif35::niaD-amdS-niaD [pDSM-JAK-120] co-transformants (Example 11) the plasmid copy number was 4 and 3-4 per genome, respectively.

To determine the copy number of plasmid pDSM-JAK-136 carrying the A. nidulans aur1 gene in P. chrysogenum DS54465 Δaur1::niaD-amdS-niaD [pDSM-JAK-<136] transformants (Example 20), we used the primers:

| | |
|---|---|
| DSM-JAK-178 | |
| 5′- GGCTGGCTGTTAGTCAACTG -3′ | (SEQ ID NO: 69) |
| and | |
| DSM-JAK-179 | |
| 5′- AGGAGGCTGACCTCGATTGT -3′ | (SEQ ID NO: 70) |

that amplify a 181 bp DNA fragment of the region of plasmid pDSM-JAK-136 comprising the A. nidulans AN0465 promoter (Example 6). Again the gene encoding gamma actin was used as a reference. Since the P_{AN0465} DNA fragment is lacking in the P. chrysogenum genome, in this case a strain carrying 4 copies of the P_{AN0465} region per genome, DS61187 Δtif35::niaD-amdS-niaD [pDSM-JAK-108] (Example 7), was used as a reference, while strain DS54465, carrying 0 copies/genome was used as negative control. We observed that pDSM-JAK-136 was present in 10 to 11 copies per genome. We presume that the higher copy number of this plasmid as compared to plasmids pDSM-JAK-108 and pDSM-JAK-120 is caused by the use of the heterologous aur1 gene, which may be less expressed than the homologous P. chrysogenum aur1 gene thereby driving an increase in copy number.

1. A host cell deficient in an essential gene, comprising a vector, said vector comprising at least said essential gene and an autonomous replication sequence, wherein said host cell is a filamentous fungal cell.

2. A host cell according to claim 1, wherein said host cell comprises a recombinant polynucleotide construct comprising a polynucleotide encoding a biological compound of interest and/or a compound involved in synthesis of a biological compound of interest.

3. A host cell deficient in an essential gene, comprising a vector, said vector comprising at least said essential gene and an autonomous replication sequence, wherein said host cell comprises a recombinant polynucleotide construct comprising a polynucleotide encoding a biological compound of interest and/or a compound involved in synthesis of a biological compound of interest.

4. A host cell according to claim 3, wherein said host cell is a eukaryotic cell.

5. A host cell according to claim 3, wherein said host cell is a fungal cell, optionally a filamentous fungal cell.

6. A host cell according to claim 1, wherein said host cell is, optionally inducibly, increased in efficiency of homologous recombination (HR), decreased in efficiency of non-homologous recombination (NHR) and/or decreased in ratio of non-homologous recombination/homologous recombination (NHR/HR).

7. A host cell according to claim 1, wherein said host cell deficiency in essential gene is inducible.

8. A host cell according to claim 1, wherein the essential gene is an essential gene in fungi.

9. A host cell according to claim 1, wherein the essential gene is the tif35 or aur1 gene.

10. A host cell according to claim 1, wherein the host cell is an Aspergillus, Chrysosporium, Penicillium, Rasamsonia, Talaromyces or Trichoderma.

11. A host cell according to claim 10, wherein the host cell is Aspergillus niger, Penicillium chrysogenum, or Rasamsonia emersonii.

12. A host cell according to claim 1, wherein the vector contains control sequences from a species other than a host species.

13. A vector-host system comprising a host cell according to claim 1.

14. A method for producing a host cell and/or of a vector-host system, which method comprises:
a. providing a host cell and a vector, which comprises at least a gene essential for said host cell and an autonomous replication sequence,
b. co-transforming the host cell with the vector and a disruption construct for said essential gene to render the host cell deficient in the essential gene.

15. A method according to claim 14, wherein the host cell is, optionally inducibly, increased in efficiency of homologous recombination (HR), decreased in efficiency of non-homologous recombination (NHR) and/or decreased in ratio of non-homologous recombination/homologous recombination (NHR/HR).

16. A method according to claim 14, wherein the host cell deficiency in the essential gene is inducible.

17. A method according to claim 14, wherein the host cell comprises a recombinant polynucleotide construct comprising a polynucleotide encoding a biological compound of interest and/or a compound involved in synthesis of a biological compound of interest.

18. A method according to claim 14, wherein the essential gene is an essential gene in fungi.

19. A method according to claim 14, wherein the essential gene is the tif35 or aur1 gene.

20. A method according to claim 14, wherein the host cell is an Aspergillus, Chrysosporium, Penicillium Saccharomyces, Rasamsonia, Talaromyces or Trichoderma.

21. A method according to claim 14, wherein the host cell is Aspergillus niger, Penicillium chrysogenum, Saccharomyces cerevisiae or Rasamsonia emersonii.

22. A method according to claim 14, wherein the vector contains control sequences from a species other than a host species.

23. A method for producing a biological compound of interest comprising culturing the vector-host system of claim 13, under conditions conducive to producing a biological compound of interest and optionally isolating a compound of interest from a culture broth.

24. A method for producing a biological compound of interest comprising producing a host cell and/or a vector-host system according to the method of claim 14, and culturing said vector-host system and/or host cell under conditions conducive to producing a biological compound of interest and optionally isolating a compound of interest from a culture broth.

25. A method for producing a biological compound of interest comprising:
a. providing a host cell, said host cell being deficient in an essential gene, said host cell comprising a vector, said vector comprising at least said essential gene and an autonomous replication sequence, wherein the host cell is (i) a filamentous fungal cell and/or (ii) a host cell which comprises a recombinant polynucleotide construct comprising a polynucleotide encoding a biological compound of interest or a compound involved in synthesis of a biological compound of interest,
b. optionally providing said host cell with a recombinant polynucleotide construct comprising a polynucleotide encoding a biological compound of interest and/or compound involved in synthesis of a biological compound of interest,
c. culturing the host cell under conditions conducive to producing a biological compound of interest, and optionally
d. isolating a biological compound of interest from a culture broth.

26. A method for producing a biological compound of interest comprising:
a. providing a host cell, said host cell being deficient in an essential gene, said host cell comprising a vector, said vector comprising at least said essential gene and an autonomous replication sequence, wherein said host cell is prepared according to the method claim 14,
b. optionally providing said host cell with a recombinant polynucleotide construct comprising a polynucleotide encoding a biological compound of interest and/or compound involved in synthesis of a biological compound of interest,
c. culturing the host cell under conditions conducive to producing a biological compound of interest, and optionally
d. isolating a biological compound of interest from a culture broth.

27. A method for screening for a polynucleotide encoding a biological compound of interest comprising:
a. providing a library of polynucleotides optionally containing a polynucleotide encoding a biological compound of interest,
b. providing a multiplicity of individual host cells, said host cell being deficient in an essential gene and comprising a vector, said vector comprising at least said essential gene and an autonomous replication sequence, wherein said host cell is (i) a filamentous fungal cell and/or (ii) a host cell which comprises a recombinant polynucleotide construct comprising a polynucleotide encoding a biological compound of interest and/or a compound involved in the synthesis of a biological compound of interest,
c. screening a transformant for expressing a biological compound of interest.

28. A method for screening for a polynucleotide encoding a biological compound of interest comprising:
a. providing a library of polynucleotides optionally containing a polynucleotide encoding a biological compound of interest,
b. providing a multiplicity of individual host cells, said host cell being deficient in an essential gene and comprising a vector, said vector comprising at least said essential gene and an autonomous replication sequence, wherein said host cell is prepared according to the method of claim 14,
c. screening a transformant for expressing a biological compound of interest.

29. The vector-host system according to claim 13, capable of being used for producing a biological compound of interest.

30. The vector-host system according to claim 13, capable of being used for expressing a polynucleotide encoding a compound of interest.

31. A host cell deficient in an essential gene capable of being used to stabilize a vector, said vector comprising at least said essential gene and an autonomous replication sequence.

32. A host cell deficient in an essential gene capable of being used according to claim 31, wherein said host cell is, optionally inducibly, increased in efficiency of homologous recombination (HR), decreased in efficiency of non-homologous recombination (NHR) and/or decreased in ratio of non-homologous recombination/homologous recombination (NHR/HR).

33. A host cell deficient in an essential gene capable of being used according to claim 31, wherein said host cell deficiency in the essential gene is inducible.

34. A host cell deficient in an essential gene capable of being used according to claim 31, wherein the host cell comprises a recombinant polynucleotide construct comprising a polynucleotide encoding a biological compound of interest and/or a compound involved in the synthesis of a biological compound of interest.

35. A host cell deficient in an essential gene capable of being used according to claim 31, wherein the essential gene is an essential gene in fungi.

36. A host cell deficient in an essential gene capable of being used according to claim 31, wherein the essential gene is the tif35 or aur1 gene.

37. A host cell deficient in an essential gene capable of being used according to claim 31, wherein said host cell is an Aspergillus, Chrysosporium, Penicillium, Saccharomyces, Rasamsonia, Talaromyces or Trichoderma.

38. A host cell deficient in an essential gene capable of being used according to claim 31, wherein the host cell is Aspergillus niger, Penicillium chrysogenum, Saccharomyces cerevisiae or Rasamsonia emersonii.

39. A host cell deficient in an essential gene capable of being used to stabilize a vector according to claim 31, wherein vector contains control sequences from a species other than a host species.

40. A host cell capable of being used to stabilize a vector-host system, said host cell being a host cell as defined in claim 31.


SEQUENCE LISTING (70 sequences)

SEQ ID NO: 1 - DNA, 55 bp, Penicillium chrysogenum
ggggacaagtttgtacaaaaaagcaggctgatcgaaggaagcagtccctacactc

SEQ ID NO: 2 - DNA, 55 bp, Penicillium chrysogenum
ggggaccactttgtacaagaaagctgggttgagactgaacaatgtgaagacggag

SEQ ID NO: 3 - DNA, 55 bp, Penicillium chrysogenum
ggggacaactttgtatagaaaagttgagcatattctttcactgttgcagatctgc

SEQ ID NO: 4 - DNA, 51 bp, Penicillium chrysogenum
ggggactgcttttttgtacaaacttgctatcccatccagatgagtgcttcg

SEQ ID NO: 5 - DNA, 53 bp, Penicillium chrysogenum
ggggacagctttcttgtacaaagtggacaccatgtctccaaccgggaagtgag

SEQ ID NO: 6 - DNA, 51 bp, Penicillium chrysogenum
ggggacaactttgtataataaagttgggtgcttgggatgttccatggtagc

SEQ ID NO: 7 - DNA, 25 bp, Penicillium chrysogenum
cagtttacactcaaccccaatccag

SEQ ID NO: 8 - DNA, 22 bp, Penicillium chrysogenum
aggttggtggagaagccattag

SEQ ID NO: 9 - DNA, 53 bp, Penicillium chrysogenum
ggggacagctttcttgtacaaagtggatgggaaactaaccacgtgcttgtacg

SEQ ID NO: 10 - DNA, 51 bp, Penicillium chrysogenum
ggggacaactttgtataataaagttgttcaccctgtctcgacttccttgtc

SEQ ID NO: 11 - DNA, 57 bp, Penicillium chrysogenum
ggggacaactttgtataataaagttgtgggccctcaccctgtctcgacttccttgtc

SEQ ID NO: 12 - DNA, 33 bp, Aspergillus nidulans
agaggtaccgagttatagacggtccggcatagg

SEQ ID NO: 13 - DNA, 33 bp, Aspergillus nidulans
agaggatccgtttgctgtctatgtgggggactg

SEQ ID NO: 14 - DNA, 27 bp, Aspergillus nidulans
ggggtgcttctaaggtatgagtcgcaa

SEQ ID NO: 15 - DNA, 35 bp, Aspergillus nidulans
agaacgcgttaacgcagggtttgagaactccgatc

SEQ ID NO: 16 - DNA, 33 bp, Penicillium chrysogenum
agaggatccgaggaagacgtgatcagagtaagc

SEQ ID NO: 17 - DNA, 42 bp, Penicillium chrysogenum
gaaagcggccgcggtaccgtgcttgggatgttccatggtagc

SEQ ID NO: 18 - DNA, 22 bp, Penicillium chrysogenum
cacgtagcatacaaccgtgtcg

SEQ ID NO: 19 - DNA, 24 bp, Penicillium chrysogenum
gatgccttgtgggaaattaaccag

SEQ ID NO: 20 - DNA, 55 bp, Penicillium chrysogenum
ggggacaagtttgtacaaaaaagcaggctgagaggaagacgtgatcagagtaagc

SEQ ID NO: 21 - DNA, 54 bp, Penicillium chrysogenum
ggggaccactttgtacaagaaagctgggttgtgcttgggatgttccatggtagc

SEQ ID NO: 22 - DNA, 24 bp, Penicillium chrysogenum
gttcttgaatagccgaggactcac

SEQ ID NO: 23 - DNA, 24 bp, Penicillium chrysogenum
catcctccccttctgttggcatag

SEQ ID NO: 24 - DNA, 37 bp, Aspergillus nidulans
agaaagcttggtaccgttgcaccaatcgccgtttagg

SEQ ID NO: 25 - DNA, 47 bp, Aspergillus nidulans
agaagatctgtcgacgaattcggtgaaggttgtgttatgttttgtgg

SEQ ID NO: 26 - DNA, 33 bp, Aspergillus nidulans
agaagatctgatcgttggtgtcgatgtcagctc

SEQ ID NO: 27 - DNA, 28 bp, Aspergillus nidulans
ggggtacacagtacacgaggacttctag

SEQ ID NO: 28 - DNA, 1545 bp, Trichoderma reesei
atgtatcggaagttggccgtcatctcggccttcttggccacagctcgtgctcagtcggcctgcactctccaatcggagactcacccgcctctgacatggcagaaatgctcgtctggtggcacgtgcactcaacagacaggctccgtggtcatcgacgccaactggcgctggactcacgctacgaacagcagcacgaactgctacgatggcaacacttggagctcgaccctatgtcctgacaacgagacctgcgcgaagaactgctgtctggacggtgccgcctacgcgtccacgtacggagttaccacgagcggtaacagcctctccattggctttgtcacccagtctgcgcagaagaacgttggcgctcgcctttaccttatggcgagcgacacgacctaccaggaattcaccctgcttggcaacgagttctctttcgatgttgatgtttcgcagctgccgtgcggcttgaacggagctctctacttcgtgtccatggacgcggatggtggcgtgagcaagtatcccaccaacaccgctggcgccaagtacggcacggggtactgtgacagccagtgtccccgcgatctgaagttcatcaatggccaggccaacgttgagggctgggagccgtcatccaacaacgcgaacacgggcattggaggacacggaagctgctgctctgagatggatatctgggaggccaactccatctccgaggctcttaccccccacccttgcacgactgtcggccaggagatctgcgagggtgatgggtgcggcggaacttactccgataacagatatggcggcacttgcgatcccgatggctgcgactggaacccataccgcctgggcaacaccagcttctacggccctggctcaagctttaccctcgataccaccaagaaattgaccgttgtcacccagttcgagacgtcgggtgccatcaaccgatactatgtccagaatggcgtcactttccagcagcccaacgccgagcttggtagttactctggcaacgagctcaacgatgattactgcacagctgaggaggcagaattcggcggatcctctttctcagacaagggcggcctgactcagttcaagaaggctacctctggcggcatggttctggtcatgagtctgtgggatgattactacgccaacatgctgtggctggactccacctacccgacaaacgagacctcctccacacccggtgccgtgcgcggaagctgctccaccagctccggtgtccctgctcaggtcgaatctcagtctcccaacgccaaggtcaccttctccaacatcaagttcggacccattggcagcaccggcaaccctagcggcggcaaccctcccggcggaaacccgcctggcaccaccaccacccgccgcccagccactaccactggaagctctcccggacctacccagtctcactacggccagtgcggcggtattggctacagcggccccacggtctgcgccagcggcacaacttgccaggtcctgaacccttactactctcagtgcctgtaa

SEQ ID NO: 29 - DNA, 4680 bp, Aspergillus niger
gggggcactgtcgatcttatcacattcacaatcatcgagctatcgcctaatatgcgtctaaaggaggaagcacccggtactggatctctatgtggaagcacctttgtcaacagacgctttgaagagatgctaaatgaccgcctctcctccctccctggctgggacagggatacactggatgaagcaatgcatcgatttgaaactgtcgctaaaagaactttcagtggaaatacagacgattacttcatgttccctgttccaggtatagcagacagccaggaagtcggggttcgtcgcggccgattccgagttactggccaagagatgcagcaactgtttttgcctatcctacgagacattgaggaccttgtccgagagcagatcgagacctctgacgctcaggtaaaagcaattttcctggttggagggtttggacagagcccataccttcgcacatatcttcgcgactgcttctctcctgaagtcgaagtgatagcaccagttgacggctggactgctgttgtcagaggcgcgttgacgaagactcttggggaggtttccgacacagagataaaaacatacgtcgattcccgaaaggcaagggaaaactatggaatgatttgttcgactagattcattgataaagtgcatgatgcaaagaagaagtaagtggcgactgcctgctcaatgtgttccagcatccgtcttccatttcttcgtcccagtggcctaataattccaggtactggaatgccaaagaaggaaagttctatattgatgttatgcattggtttgtttccaaggtatggaccgatattcgaacacacttctggtctttgataacaagttcagggggacgatatcgaagaagcgaaggccattaagacgaactggtctcagcataagcttgccaaggacggcacattcgactcaatccgcgtcaatctctacaggctcgatactcctatgggtgagaagccaccgttgtacttcaatcgccgtgagtactctcgtgtagctgattatggatgatgattatgagttattgacatttcgttcttagacgtgaagcaacatgccaagttgaatccaatccttaatcagattgaaaagaatcgcatcccaatctgccatggtgcagataacgagctctactacacgatcggatttcaaatccatgcggtgtactattccgctcactgtgaatacatgttctggtatgaaggctgcaatcatggaagcgtcaaagccgaatacgtttgaccgttaacctttatgagctttcctttggacattatcctaccaccatatctcagtgacaaaaactagtcaatcgtttgctaaggagcttagaagtaggacgaaaagaagaatggttacccattgaaggacgaacacttgcgttaggcgttgtcgacattctgatctattttagcagcgagaaatctagataaccattgagtcaattctccaactaatatgctgtctaatatgtatctaaagttaaagaaaatctacaaaaagacctgcttgatgataaaatgacgttcgatgcatccataccaattgtagccattgtcccgtgtatcacgtgatccaggcaccgaagcaccagccacagattgcatgcgccacaggccatttgccgcgccgaacaaatccacaacggatcttgcatctttcgctgaaatccagaaattcagcaacaatcggcgacatcatcaacccccccccatcacacgcacaatgtcgaagcttggaaagtaagcggaaactccccacccccaaaaaaccagggccacgaagcgacaaaaaagaagaaaaagaaagaaagaaaatctgaccaggacctttttccactttccagccgcgccgactgggccgacgacgaggagttcgacgacccctccgctctccccccgcagcaaatcacgaccaacaaagatggcacgaagacgatcgtctcctaccgattcaacgacgaaggcaagaaggtgaaggtgacccgccggatcaaaacgaccgttgtgcgcgaacatgtcaacccgcaggtcgcggagcggaggtcctgggccaagttcggtctggagaagggcaacgcgcccggaccttcgttcgatacgacctccgtgggtgagaacattgttttccggcccagcgtcaactggaagcttcaggcggctgaggcggagaagaacggtggcgagaagggcagtgtgaaggatcagctgaaggacaagaaggtcaagtgtcgtatttgcagtggcgagcactttactgctcgctgtcccttcaaggatactatggctcctgtcgacgagcccactgctggtggagaggctggtgatgaggattctccggctgctggcgctttgggtgctggtacttctagctacgtgccccctcatctgcggaagggtgctgctggtggcggagagagaatggctggcaagtatgagaaggatgatttggcgactctgagagttacgaacgtgagttttcccatcccctccttgttttgatttactgttctgtgtttggctatttgctggtctccccggaggggttgggggaggggtcgagggacaggggatgctagttttcctctttctgtctctttctctcctcctttcccttgcctgctctgccctccctttgccctctaaacactcctattggtgtcgttcagagggagaaagaaagaggcagcagccctttcgggcagacacatggatgtcatgagaacagcaatcagcttcctctaatttccttgcttctattgcatttgcttcccacggacacatcactgactgacaataaacccaaacaggtgagcgagttggcagaggaaggagaactgcgggatctgttcgaacgcttcggtcgtgtcaccagagtcttccttgccagagacagagaaacccagagagccaagggcttcgctttcatcagctttgcggatcggagcgatgctgcacgtgcttgcgagaagatggatggctgtaagttatccctcccggttacgtttccatttttcgtcatattctgttttgtttttttccgccatcgttgcaatgtcagcagcaaagcaagttatcggaatcgcaactaacatttcctcttcatctagtcggttaccgtcaccttatcctgcgcgtcgaattcgccaagcgtgccacttagatttctccaatttccaattcccattttttcttcccattttattctcctccttattaacttcttcttttgcttcttggttttccgtacgtatcatgacatgacacccttgggtctcttttcgtctggcaggcgggttctccatccatccacactttccatggctgaagaagatcttcatctcatctattgactacttttctttgtactgtatgatccgatcgataatgatgattactgaagaaaaaaaatgggggaggacatcccaatgtgtgtgcgtgtgtcgtcttctctttgctttttgggtcgatttgatacgaagaaaagcagcgtggtctatttgtcttattcacgcactactcttttccggttctatgtgtctatttatcatgctgagttgcaagtcaaggcaaggcaaggcaagcttgcttgcaagccctcttttccttgtttacttttccgtgcattgatcgatcgcgggtggaagtatacttagatcgagggagaaggttcaaccccaaaaaacaaaagtatttcaatttgctcgttcgagtcacactacatggagtagagtagttaggtaaaggttcggtcctgcttacttgggaacctagtagtgctgtccaggttctaggacggggaggaacaggttttattggctattggggtaatgctccgtattatcgtccgttcagtactgacagcctggctggggtctacttttcaatcatatggtggatgttgtagagcctcttgcttggtatatctcgggtaattggttattagatccttgctaagatgactcgatgcgtattagctgacaagctcctctgatttggcggcatgtgtcaggaaggcctggatttgagccacccgggacgcgacaaggcagcattgactgtgctgatcaggctttgcagagacgagatgcatggtggataatgaccatgggtctccagagtgcggaccaacgagccaagatcacggtcagtgtgtaggtggttcgggggtcaacagctagctgtctagggacgtcagggatgccgggacactgatcatgacaataccgaagggtgcctgtgtagtcgtttggtaaagatggaaggctggctagtagcgtttttgtatgagcttcgtcatgagtgtcatcggcaaggcagctctgcatgtgtgtctggctagggaagaaaatcggcatagcaagcgggaacgacgcattgctcgagtagacgactcaaccttgtatggagagcatgaccagatgtgagatcgatatcgagccgcccatgacctccgggctagaggacggcttctcctagcatacagatggatgcacgggactaatcagagcgtggcgccaggccagtggtttcgatctccagtcaaaatcaatttggcttcgtgtaatcctcgttcacagcccggggctttcgctgctcggaaggattgtgaatggttccgtgtgtccagatggctggttacattgcatgaaaacgggta

SEQ ID NO: 30 - DNA, 1437 bp, Aspergillus niger
atgtcgaagcttggaaagtaagcggaaactccccacccccaaaaaaccagggccacgaagcgacaaaaaagaagaaaaagaaagaaagaaaatctgaccaggacctttttccactttccagccgcgccgactgggccgacgacgaggagttcgacgacccctccgctctccccccgcagcaaatcacgaccaacaaagatggcacgaagacgatcgtctcctaccgattcaacgacgaaggcaagaaggtgaaggtgacccgccggatcaaaacgaccgttgtgcgcgaacatgtcaacccgcaggtcgcggagcggaggtcctgggccaagttcggtctggagaagggcaacgcgcccggaccttcgttcgatacgacctccgtgggtgagaacattgttttccggcccagcgtcaactggaagcttcaggcggctgaggcggagaagaacggtggcgagaagggcagtgtgaaggatcagctgaaggacaagaaggtcaagtgtcgtatttgcagtggcgagcactttactgctcgctgtcccttcaaggatactatggctcctgtcgacgagcccactgctggtggagaggctggtgatgaggattctccggctgctggcgctttgggtgctggtacttctagctacgtgccccctcatctgcggaagggtgctgctggtggcggagagagaatggctggcaagtatgagaaggatgatttggcgactctgagagttacgaacgtgagttttcccatcccctccttgttttgatttactgttctgtgtttggctatttgctggtctccccggaggggttgggggaggggtcgagggacaggggatgctagttttcctctttctgtctctttctctcctcctttcccttgcctgctctgccctccctttgccctctaaacactcctattggtgtcgttcagagggagaaagaaagaggcagcagccctttcgggcagacacatggatgtcatgagaacagcaatcagcttcctctaatttccttgcttctattgcatttgcttcccacggacacatcactgactgacaataaacccaaacaggtgagcgagttggcagaggaaggagaactgcgggatctgttcgaacgcttcggtcgtgtcaccagagtcttccttgccagagacagagaaacccagagagccaagggcttcgctttcatcagctttgcggatcggagcgatgctgcacgtgcttgcgagaagatggatggctgtaagttatccctcccggttacgtttccatttttcgtcatattctgttttgtttttttccgccatcgttgcaatgtcagcagcaaagcaagttatcggaatcgcaactaacatttcctcttcatctagtcggttaccgtcaccttatcctgcgcgtcgaattcgccaagcgtgccacttag

SEQ ID NO: 31 - DNA, 867 bp, Aspergillus niger
atgtcgaagcttggaaaccgcgccgactgggccgacgacgaggagttcgacgacccctccgctctccccccgcagcaaatcacgaccaacaaagatggcacgaagacgatcgtctcctaccgattcaacgacgaaggcaagaaggtgaaggtgacccgccggatcaaaacgaccgttgtgcgcgaacatgtcaacccgcaggtcgcggagcggaggtcctgggccaagttcggtctggagaagggcaacgcgcccggaccttcgttcgatacgacctccgtgggtgagaacattgttttccggcccagcgtcaactggaagcttcaggcggctgaggcggagaagaacggtggcgagaagggcagtgtgaaggatcagctgaaggacaagaaggtcaagtgtcgtatttgcagtggcgagcactttactgctcgctgtcccttcaaggatactatggctcctgtcgacgagcccactgctggtggagaggctggtgatgaggattctccggctgctggcgctttgggtgctggtacttctagctacgtgccccctcatctgcggaagggtgctgctggtggcggagagagaatggctggcaagtatgagaaggatgatttggcgactctgagagttacgaacgtgagcgagttggcagaggaaggagaactgcgggatctgttcgaacgcttcggtcgtgtcaccagagtcttccttgccagagacagagaaacccagagagccaagggcttcgctttcatcagctttgcggatcggagcgatgctgcacgtgcttgcgagaagatggatggcttcggttaccgtcaccttatcctgcgcgtcgaattcgccaagcgtgccacttag

SEQ ID NO: 32 - protein, 288 aa, Aspergillus niger
MSKLGNRADWADDEEFDDPSALPPQQITTNKDGTKTIVSYRFNDEGKKVKVTRRIKTTVVREHVNPQVAERRSWAKFGLEKGNAPGPSFDTTSVGENIVFRPSVNWKLQAAEAEKNGGEKGSVKDQLKDKKVKCRICSGEHFTARCPFKDTMAPVDEPTAGGEAGDEDSPAAGALGAGTSSYVPPHLRKGAAGGGERMAGKYEKDDLATLRVTNVSELAEEGELRDLFERFGRVTRVFLARDRETQRAKGFAFISFADRSDAARACEKMDGFGYRHLILRVEFAKRAT

SEQ ID NO: 33 - DNA, 6182 bp, Aspergillus niger
cggccgcgggggcactgtcgatcttatcacattcacaatcatcgagctatcgcctaatatgcgtctaaaggaggaagcacccggtactggatctctatgtggaagcacctttgtcaacagacgctttgaagagatgctaaatgaccgcctctcctccctccctggctgggacagggatacactggatgaagcaatgcatcgatttgaaactgtcgctaaaagaactttcagtggaaatacagacgattacttcatgttccctgttccaggtatagcagacagccaggaagtcggggttcgtcgcggccgattccgagttactggccaagagatgcagcaactgtttttgcctatcctacgagacattgaggaccttgtccgagagcagatcgagacctctgacgctcaggtaaaagcaattttcctggttggagggtttggacagagcccataccttcgcacatatcttcgcgactgcttctctcctgaagtcgaagtgatagcaccagttgacggctggactgctgttgtcagaggcgcgttgacgaagactcttggggaggtttccgacacagagataaaaacatacgtcgattcccgaaaggcaagggaaaactatggaatgatttgttcgactagattcattgataaagtgcatgatgcaaagaagaagtaagtggcgactgcctgctcaatgtgttccagcatccgtcttccatttcttcgtcccagtggcctaataattccaggtactggaatgccaaagaaggaaagttctatattgatgttatgcattggtttgtttccaaggtatggaccgatattcgaacacacttctggtctttgataacaagttcagggggacgatatcgaagaagcgaaggccattaagacgaactggtctcagcataagcttgccaaggacggcacattcgactcaatccgcgtcaatctctacaggctcgatactcctatgggtgagaagccaccgttgtacttcaatcgccgtgagtactctcgtgtagctgattatggatgatgattatgagttattgacatttcgttcttagacgtgaagcaacatgccaagttgaatccaatccttaatcagattgaaaagaatcgcatcccaatctgccatggtgcagataacgagctctactacacgatcggatttcaaatccatgcggtgtactattccgctcactgtgaatacatgttctggtatgaaggctgcaatcatggaagcgtcaaagccgaatacgtttgaccgttaacctttatgagctttcctttggacattatcctaccaccatatctcagtgacaaaaactagtcaatcgtttgctaaggagcttagaagtaggacgaaaagaagaatggttacccattgaaggacgaacacttgcgttaggcgttgtcgacattctgatctattttagcagcgagaaatctagataaccattgagtcaattctccaactaatatgctgtctaatatgtatctaggcgcgccctcgcacgcatgggttgagtggtatggggccatccagagtcacctgtggcagcatgagactgcactcgaagcagccatcaacccagccaatattctgggctttccatccttagatcacatttgagatataacccatttggtgagagacacttgtgccgttatacgtgtctagactggaaacgcaaccctgaagggattcttcctttgagagatggaagcgtgtcatatctcttcggttctacggcaggtttttttctgctctttcgtagcatggcatggtcacttcagcgcttatttacagttgctggtattgatttcttgtgcaaattgctatctgacacttattagctatggagtcaccacatttcccagcaacttccccacttcctctgcaatcgccaacgtcctctcttcactgagtctccgtccgataacctgcactgcaaccggtgccccatggtacgcctccggatcatactcttcctgcacgagggcatcaagctcactaaccgccttgaaactctcattcttcttatcgatgttcttatccgcaaaggtaaccggaacaaccacgctcgtgaaatccagcaggttgatcacagaggcatacccatagtaccggaactggtcatgccgtaccgcagcggtaggcgtaatcggcgcgatgatggcgtccagttccttcccggccttttcttcagcctcccgccatttctcaaggtactccatctggtaattccacttctggagatgcgtgtcccagagctcgttcatgttaacagctttgatgttcgggttcagtaggtctttgatatttggaatcgccggctcgctggatgcactgatatcgcgcattacgtcggcgctgccgtcagccgcgtagatatgggagatgagatcgtggccgaaatcgtgcttgtatggcgtccacgaggtcacggtgtgaccggctttggcgagtgcggcgacggtggtttccacgccgcgcaggataggagggtgtggaaggacattgccgtcgaagttgtagtagccgatattgagcccgccgttcttgatcttggaggcaataatgtccgactcggactggcgccagggcatggggatgaccttggagtcgtatttccatggctcctgaccgaggacggatttggtgaagaggcggaggtctaacatacttcatcagtgactgccggtctcgtatatagtataaaaagcaagaaaggaggacagtggaggcctggtatagagcaggaaaagaaggaagaggcgaaggactcaccctcaacagagtgcgtaatcggcccgacaacgctgtgcaccgtctcctgaccctccatgctgttcgccatctttgcatacggcagccgcccatgactcggccttagaccgtacaggaagttgaacgcggccggcactcgaatcgagccaccgatatccgttcctacaccgatgacgccaccacgaatcccaacgatcgcaccctcaccaccagaactgccgccgcacgaccagttcttgttgcgtgggttgacggtgcgcccgatgatgttgttgactgtctcgcagaccatcagggtctgcgggacagaggtcttgacgtagaagacggcaccggctttgcggagcatggttgtcagaaccgagtccccttcgtcgtacttgtttagccatgagatgtagcccattgatgtttcgtagccctggtggcatatgttagctgacaaaaagggacatctaacgacttaggggcaacggtgtaccttgactcgaagctggtctttgagagagatggggaggccatggagtggaccaacgggtctcttgtgctttgcgtagtattcatcgagttcccttgcctgcgcgagagcggcgtcagggaagaactcgtgggcgcagtttgtctgcacagaagccagcgtcagcttgatagtcccataaggtggcgttgttacatctccctgagaggtagaggggaccctactaactgctgggcgattgctgcccgtttacagaatgctagcgtaacttccaccgaggtcaactctccggccgccagcttggacacaagatctgcagcggaggcctctgtgatcttcagttcggcctctgaaaggatccccgatttctttgggaaatcaataacgctgtcttccgcaggcagcgtctggactttccattcatcagggatggtttttgcgaggcgggcgcgcttatcagcggccagttcttcccaggattgaggcattgtgatgtctgctcaagcggggtagctgttagtcaagctgcgatgaagtgggaaagctcgaactgaaaggttcaaaggaataagggatgggaaggatggagtatggatgtagcaaagtacttacttaggggaaataaaggttcttggatgggaagatgaatatactgaagatgggaaaagaaagagaaaagaaaagagcagctggtggggagagcaggaaaatatggcaacaaatgttggactgacgcaacgaccttgtcaaccccgccgacacaccgggcggacagacggggcaaagctgcctaccagggactgagggacctcagcaggtcgagtgcagagcaccggatgggtcgactgccagcttgtgttcccggtctgcgccgctggccagctcctgagcggcctttccggtttcatacaccgggcaaagcaggagaggcacgatatttggacgccctacagatgccggatgggccaattagggagcttacgcgccgggtactcgctctacctacttcggagaaggtactatctcgtgaatcttttaccagatcggaagcaattggacttctgtacctaggttaatggcatgctatttcgccgacggctatacacccctggcttcacattctccttcgcttactgccggtgattcgatgaagctccatattctccgatgatgcaatagattcttggtcaacgaggggcacaccagcctttccacttcggggcggaggggcggccggtcccggattaataatcatccactgcacctcagagccgccagagctgtctggcgcagtggcgcttattactcagcccttctctctgcgtccgtccgtctctccgcatgccagaaagagtcaccggtcactgtacagagctcgaattggacgctgaggccggcctagatttctccaatttccaattcccattttttcttcccattttattctcctccttattaacttcttcttttgcttcttggttttccgtacgtatcatgacatgacacccttgggtctcttttcgtctggcaggcgggttctccatccatccacactttccatggctgaagaagatcttcatctcatctattgactacttttctttgtactgtatgatccgatcgataatgatgattactgaagaaaaaaaatgggggaggacatcccaatgtgtgtgcgtgtgtcgtcttctctttgctttttgggtcgatttgatacgaagaaaagcagcgtggtctatttgtcttattcacgcactactcttttccggttctatgtgtctatttatcatgctgagttgcaagtcaaggcaaggcaaggcaagcttgcttgcaagccctcttttccttgtttacttttccgtgcattgatcgatcgcgggtggaagtatacttagatcgagggagaaggttcaaccccaaaaaacaaaagtatttcaatttgctcgttcgagtcacactacatggagtagagtagttaggtaaaggttcggtcctgcttacttgggaacctagtagtgctgtccaggttctaggacggggaggaacaggttttattggctattggggtaatgctccgtattatcgtccgttcagtactgacagcctggctggggtctacttttcaatcatatggtggatgttgtagagcctcttgcttggtatatctcgggtaattggttattagatccttgctaagatgactcgatgcgtattagctgacaagctcctctgatttggcggcatgtgtcaggaaggcctggatttgagccacccgggacgcgacaaggcagcattgactgtgctgatcaggctttgcagagacgagatgcatggtggataatgaccatgggtctccagagtgcggaccaacgagccaagatcacggtcagtgtgtaggtggttcgggggtcaacagctagctgtctagggacgtcagggatgccgggacactgatcatgacaataccgaagggtgcctgtgtagtcgtttggtaaagatggaaggctggctagtagcgtttttgtatgagcttcgtcatgagtgtcatcggcaaggcagctctgcatgtgtgtctggctagggaagaaaatcggcatagcaagcgggaacgacgcattgctcgagtagacgactcaaccttgtatggagagcatgaccagatgtgagatcgatatcgagccgcccatgacctccgggctagaggacggcttctcctagcatacagatggatgcacgggactaatcagagcgtggcgccaggccagtggtttcgatctccagtcaaaatcaatttggcttcgtgtaatcctcgttcacagcccggggctttcgctgctcggaaggattgtgaatggttccgtgtgtccagatggctggttacattgcatgaaaacgggtagcggccgc

SEQ ID NO: 34 - DNA, 714 bp, Discosoma sp.
atggcctcctccgaggacgtcatcaaggagttcatgcgcttcaaggtgcgcatggagggctccgtgaacggccacgagttcgagatcgagggcgagggcgagggccgcccctacgagggcacccagaccgccaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggtgaccgtgacccaggactcctccctgcaggacggctccttcatctacaaggtgaagttcatcggcgtgaacttcccctccgacggccccgtaatgcagaagaagactatgggctgggaggcctccaccgagcgcctgtacccccgcgacggcgtgctgaagggcgagatccacaaggccctgaagctgaaggacggcggccactacctggtggagttcaagtccatctacatggccaagaagcccgtgcagctgcccggctactactacgtggactccaagctggacatcacctcccacaacgaggactacaccatcgtggagcagtacgagcgcgccgagggccgccaccacctgttcctgactcacggtatggacgaattgtacaagtcgaagctgtaa

SEQ ID NO: 35 - protein, 237 aa, Discosoma sp.
MASSEDVIKEFMRFKVRMEGSVNGHEFEIEGEGEGRPYEGTQTAKLKVTKGGPLPFAWDILSPQFQYGSKVYVKHPADIPDYKKLSFPEGFKWERVMNFEDGGVVTVTQDSSLQDGSFIYKVKFIGVNFPSDGPVMQKKTMGWEASTERLYPRDGVLKGEIHKALKLKDGGHYLVEFKSIYMAKKPVQLPGYYYVDSKLDITSHNEDYTIVEQYERAEGRHHLFLTHGMDELYKSKL

SEQ ID NO: 36 - DNA, 2968 bp, Discosoma sp.
gagttatagacggtccggcataggtaaggagagattagatatttcgacttcccgattccttagtctccttagtctggaaactgagaaaggcctgaactcgttcaatctattagctctaatcacgtagtctcaactagaatcctgaacaggcgccggaaccagtccaaatgcgtggagtacagcaagtccagtcaaatcacgattagaatggccatatataagcaaaaaacccttggacaatacgtccgatcgagaccaaagcaatctggtacatcacccttcttatatacagggtagccggaattgtattgactctatacgaagccgcaggtttaccagcacagaggtgcgatagctacacgaatgtagagtgaaggcattacaaccgattgctaagtgtcatagaggctaagcttcacggctcaaggagtctgtggaggagacctgatataagaattgagagttatactccggaggttatgttagtacctcaaaaatgtgaagtacaacgaaactcgaacttcgaatgacaatagcttggctggctgttagtcaactgaggtacaacgtcatcttgcaattagttatgttctttgctagatagtgagctgctgtaaacttgtagttcttagaaacatgtctgaagtctagagtcgagacttgtgattggtgtaaattagtgggggtccggagcggaataacaatcgaggtcagcctcctgggtctgtcagtaagctaaccacgaaaggacaagcgtagacgaggtcacggggtactggcctagccggcttcggctagcgcccacgaaaatagggcaacgtcgaagcgagagacacacacggattccacgatcgccgacgacaagcactcaactccacgacaacccacgacaacccttgccgctgcgttcttggtgaagggctgcaggattagaggtgagtattttattttctggctattgtgatctttttcccccccggtttccgctggttctatcttgtcgagcgagtcggtgcgccttcgatcggtctgtcaaatttccatccgtccactcgttcacccggtatcgatacaagagggaatggagcattacgacaggggagcagaggcagaactctcgatgttgctactttttttgcttggaacaacttccttgaaagattcttttctgaatgcggctgggaaactgcgaattggatatccagcgaaaggttaccgacagactcacggccatcgataccactcagcgggatagaattgaaggcattcggacattagatgatcagcttcgcttgtttcgactcgtgctaatcaattatccctggacagtcccccacatagacagcaaacggatccatggcctcctccgaggacgtcatcaaggagttcatgcgcttcaaggtgcgcatggagggctccgtgaacggccacgagttcgagatcgagggcgagggcgagggccgcccctacgagggcacccagaccgccaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggtgaccgtgacccaggactcctccctgcaggacggctccttcatctacaaggtgaagttcatcggcgtgaacttcccctccgacggccccgtaatgcagaagaagactatgggctgggaggcctccaccgagcgcctgtacccccgcgacggcgtgctgaagggcgagatccacaaggccctgaagctgaaggacggcggccactacctggtggagttcaagtccatctacatggccaagaagcccgtgcagctgcccggctactactacgtggactccaagctggacatcacctcccacaacgaggactacaccatcgtggagcagtacgagcgcgccgagggccgccaccacctgttcctgactcacggtatggacgaattgtacaagtcgaagctgtaagtcgactctagaggatcgatccccggggtgcttctaaggtatgagtcgcaaaattgttttttatttttggtcttgagtctaatatgctcgcagctcttgcgttgtatatggtcgttgtcgcgtattttctgttgtattaaaagatcaaacgagatcaagggatggctcgcgggctgtctctcgcactaggaggaagaatgcctgaaaaaggaactttgattttagctgtggaatagagatggcttgtttgaggacgcttgtcgcttggcgcagggacttgaatggcagcttgtggaaaccgaaggcgagaaaagtcgacggatactgtacgtggttctattgccagtgcggtggaagcttccttctcatatagttcaatccttctttgaatctgtttgtttcatatttggactgtttcattctctgcttgcgcattctcatcttcgagaacgactgcagggattgttggttctgtggagctgatgagcgcgccttgaccacccttgttcttgttttgctcttttgttctcatttaacccgtttctcccttccaaccctttgaccttgcaacattgtctcccagcgcgttgccaaagcgaacttgatatcagtatagtatgaccaagtagtctaccaaaataaattttagtacagtattgctagttatacaagataaattttgtacaacaatgattatgcagagctggtattctgtaaggagtaaattcatgcaggtacagagcgtccacgtcaggcacacgcgctttagtgctgggtacacattatgctacctttggggcaaggtgctattctaggtagggtatgcctagcaatgccatgatcttatgacctcagtaatctgaccttggatttaagatcggagttctcaaaccctgcg

SEQ ID NO: 37 - DNA, 3009 bp, Penicillium chrysogenum
gaggaagacgtgatcagagtaagccctatgccaacagaaggggaggatgggatcagagtaagacccatgtcagattctgcaaacgaaggacggccgaaattacttgccgcccccccccggtgtatgtggcctaagagaagacacagtgtcttagacaatcatattcccagctcgtccatcatctcatcgacatccttgattggagcatacataccggaatccaatcagtgaattatgaatgacacaaccactcagtttcccacgattcatggtgtcggcaatcatctcccgggcgcaatctcgccatgggatactggatggagagataaccgagattcggccggcttcacttcaacttgcatggctctgatgcaagatgtgtgcggatcagatctcctagccaaaggcccgcacattcaacgtcgtgactggcgtggcgcgaaggctatcaaccttgttgaccagatcccctcttgtcatcgagaccggcagagaagggagcgtctcttgtcgaacaatccggtcatggcattgagagccagaagacataggggttgaatggcccctaagttgaccaaaaatgtgagtgtatcgatgtttcagacagcaatgccgattcgtcaagctgaagaacccttggaagcttcaaggacctgtcaggttgcatctctctcggacgtgacccagtctcagtcatcgttagaataggaagaagttcagccctcaggtcaaggatggttaatgcagtcaggtctcgttggcctaaatgatagttatgtacgccacggtattcttgtagctggccttgaagcttcagaagactgcccacgaaattagcagtatcaagttcggcgcccggacttcccatttgggttctgtgataatttctttgatacgacagtcgttaggagccgattttttcttcctattctttttcccctgctgtgtaggttggatgttttcgtgagggatgttggcctgaaagagttgaatgtcaaatcaatcactatcgttcgactgttcacccatgatgtcctccagctcgcccatccggcgcgtcttcgaaacggtgtgcttctgagcggccctctgagaggccgatgtacttagaaacatctcccaataatctggacctttaaccgtgaatgtttcacaaccttgatcagtttaaatgataaatagtgacacaaggtgaagtacgagcccggagatcagggcgaatgactactctgcccgctataagtattgggtttggtccgaaggaactgtgttcggggcctttaaagttaaatatttttgaaagcagcgcttgcagagagtgttaatattcggaccccgttgaatgtcatgtttatcattgggcaaacagtggagtggtgtcaatcttgtatcagtctagtagaaacgaggttaaagaaccttggaactactttagatggggctatacacaccgcgccacgtgattcccgccaccagccacaactgtgccacagtaaattattaaatccacaacggattcgcctttgaaagaaatccagaaattcagcagccgtaataacaccatcacttacaccatgtctccaaccgggaagtgagtatttgcagttccagtcctacaagaaacacgcgcctaacaaaactttctattttacagccgcgccgactgggccgacgacgaagatttcgatgacccctccgtgctcccgccgcaagaagtgatcacgaacaaggacggcaccaagaccgtcatttcctaccgctacaatgatgacaacaagaaagtgaaggtcacccgccgaattaagaccaccgttgtccgcgaacatgtcaacccccaagtcgccgaacgccgcaaatgggagaagttcggtctcgagaagggccacgccgccggtccctcgttcgacaccacctccgtcggcgagaacatcatcttccgccccagcgtcaactggaaggctaatgctaaggaggccgagaaggagggtggcgaaaagggcagcatgaaggaccagttgaaggacaagaaggtcaagtgtcgtatttgcagtggagagcatttcacggctcgctgtcccttcaaggacaccatggctcccgtcgaggagggcactgctgctgctcctggtgtcgaggcagaggaggatgcaggtggtcttggtgctggaaagtccagctacgttcctcctcacatgcggaagggcggtgctggtggcggcgagaagatgggcggtcgtttcgagaaggacgatttggcgactctcagagttacaaacgtatgttctgccatttttctgctttctacgggctctacgggcgtcagggtcgtttattttggttcggtattgacttttcaaacaggtcagcgagttggcagaggagaacgagttgcgggatctcttcgagcgtttcggtcgtgtcaccagagtcttccttgcacgggatcgggaaacccagagagccaagggctttgctttcatcagctatgcggaccgtggcgacgcagcacttgcttgcgagaaggtggatggctgtatgtcttcaataccccatttcgtgctttcctttctaccagttcctcaagtactaacccgtctttctgctagtcggttaccgccaccttattctccgcgtcgagttcgccaagcgcactacttaaacttctttatcggttctctcttacgactttttgaatggaacgtttccttcttctcaggcgggcctatctttgggccgaagctcttttccttgtactgtaggacctggttgataatgattcccaaaaagacatccagcatgtcagttacttgcattcgtcagtctatacaaaagcaatggtttagagaaattttgaactttatacatggttttatttgttgcttcacggccgtaccttctggaaatccacggtaggagtgtcaatttgcgtttttgataatccttccaaggttcttctcgaagtagttgttctataattgcttcacagctaccatggaacatcccaagcac

SEQ ID NO: 38 - DNA, 1085 bp, Penicillium chrysogenum
atgtctccaaccgggaagtgagtatttgcagttccagtcctacaagaaacacgcgcctaacaaaactttctattttacagccgcgccgactgggccgacgacgaagatttcgatgacccctccgtgctcccgccgcaagaagtgatcacgaacaaggacggcaccaagaccgtcatttcctaccgctacaatgatgacaacaagaaagtgaaggtcacccgccgaattaagaccaccgttgtccgcgaacatgtcaacccccaagtcgccgaacgccgcaaatgggagaagttcggtctcgagaagggccacgccgccggtccctcgttcgacaccacctccgtcggcgagaacatcatcttccgccccagcgtcaactggaaggctaatgctaaggaggccgagaaggagggtggcgaaaagggcagcatgaaggaccagttgaaggacaagaaggtcaagtgtcgtatttgcagtggagagcatttcacggctcgctgtcccttcaaggacaccatggctcccgtcgaggagggcactgctgctgctcctggtgtcgaggcagaggaggatgcaggtggtcttggtgctggaaagtccagctacgttcctcctcacatgcggaagggcggtgctggtggcggcgagaagatgggcggtcgtttcgagaaggacgatttggcgactctcagagttacaaacgtatgttctgccatttttctgctttctacgggctctacgggcgtcagggtcgtttattttggttcggtattgacttttcaaacaggtcagcgagttggcagaggagaacgagttgcgggatctcttcgagcgtttcggtcgtgtcaccagagtcttccttgcacgggatcgggaaacccagagagccaagggctttgctttcatcagctatgcggaccgtggcgacgcagcacttgcttgcgagaaggtggatggctgtatgtcttcaataccccatttcgtgctttcctttctaccagttcctcaagtactaacccgtctttctgctagtcggttaccgccaccttattctccgcgtcgagttcgccaagcgcactacttaa

SEQ ID NO: 39 - DNA, 864 bp, Penicillium chrysogenum
atgtctccaaccgggaaccgcgccgactgggccgacgacgaagatttcgatgacccctccgtgctcccgccgcaagaagtgatcacgaacaaggacggcaccaagaccgtcatttcctaccgctacaatgatgacaacaagaaagtgaaggtcacccgccgaattaagaccaccgttgtccgcgaacatgtcaacccccaagtcgccgaacgccgcaaatgggagaagttcggtctcgagaagggccacgccgccggtccctcgttcgacaccacctccgtcggcgagaacatcatcttccgccccagcgtcaactggaaggctaatgctaaggaggccgagaaggagggtggcgaaaagggcagcatgaaggaccagttgaaggacaagaaggtcaagtgtcgtatttgcagtggagagcatttcacggctcgctgtcccttcaaggacaccatggctcccgtcgaggagggcactgctgctgctcctggtgtcgaggcagaggaggatgcaggtggtcttggtgctggaaagtccagctacgttcctcctcacatgcggaagggcggtgctggtggcggcgagaagatgggcggtcgtttcgagaaggacgatttggcgactctcagagttacaaacgtcagcgagttggcagaggagaacgagttgcgggatctcttcgagcgtttcggtcgtgtcaccagagtcttccttgcacgggatcgggaaacccagagagccaagggctttgctttcatcagctatgcggaccgtggcgacgcagcacttgcttgcgagaaggtggatggcttcggttaccgccaccttattctccgcgtcgagttcgccaagcgcactacttaa

SEQ ID NO: 40 - protein, 287 aa, Penicillium chrysogenum
MSPTGNRADWADDEDFDDPSVLPPQEVITNKDGTKTVISYRYNDDNKKVKVTRRIKTTVVREHVNPQVAERRKWEKFGLEKGHAAGPSFDTTSVGENIIFRPSVNWKANAKEAEKEGGEKGSMKDQLKDKKVKCRICSGEHFTARCPFKDTMAPVEEGTAAAPGVEAEEDAGGLGAGKSSYVPPHMRKGGAGGGEKMGGRFEKDDLATLRVTNVSELAEENELRDLFERFGRVTRVFLARDRETQRAKGFAFISYADRGDAALACEKVDGFGYRHLILRVEFAKRTT

SEQ ID NO: 41 - DNA, 7890 bp, Rasamsonia emersonii
ggagcctggctcagcatgctcacggactgcaggatgaacacgcgtctccgaaggtgcaaccggggagaagataaccaccgtccttgagggaagaacatctgtcatcatcgaaacagaagcagtctcagtctcggtctcggtctcagtcccgtcatctatagtcgtcacagttgggatcggaaacgtggccgaccagtccctggtcttgtaccaggtgttattctcgtatcgcggcggcgcgatcatgtcgagcgcctcaccaatccttccgaggacatagcccatcctgaacccgccctggaggcgctcgatgaagttgttggcgggatgaacggcggtgaaagggatggcatggaagatgccgaggatggccatggccatggagaacatgaacaggccgaaggggtccatggtgatgatgattcgatcgatatcagacagctgaatatgtattagctttccaaatttcttctatcatgaaattcgacaggaaatagaaaaagaaaggatgctggctttgattaaagagagtagctgggtcttatcacaagagattaaaaagtgtcttaaacaaagacaagacaagacaagcatccgtctgatgcatgagtttcgagatatatataatacaaaggatggattcactgaacagttcgttgacttctgaagaacaaaggctggtctctgcatgcccgagaatagtaacaacaataaagagcaaatacaacgttgtgtacagcaagcgaatgacctgccttgaatgagtctggcagatttattcccccatctactaccttcaagtacctcatcagatggccagcagaaggtgcaagtgggtatatcttctcattcgaagaacttagtgtttagttttctgagcagcaatatagctagttgcaagttagaaaagagtataaagaaccgtttccgcaacaccagctaccctcaaagaaaggaattgaacaaactgggaactaccgattactaccaagctggagggcataggcaatgaacgccaggaattggtaaagactgaagataggaaggtatggtgaatactgcatagtgcaatgtagaccttcagtatcataaggatcatccattcattatagacgctaatataaagtatttctgaaaaaaaatgttggagaggagatcaagtctttttattcaacggctttcaaagactaaaagctggaaaaggcaggctatcatcatgatacctagcatagcataccatgtttgcgtttatcacttcatattcaggacaacctcctgcccaggaccacgccataggcaactgtcgccaggagaccgcccaggttgaccagcgtcgagattccatgcagcttggcaaacttcttgttgagctcggtcatctccttggagtgaggaggcgggtcataactcttcttgccgtcgatcgattctgccagagatcatatatatcagcccgtacctatacagtgtactcgtgcgtactcggcagcagccaaaccggtaggtagtaggcaggtaccaaccttgctgccacctctgcttgataactcccacaaccttcggggtcagatagagcaggttggtcagtcccgaaacgaagacgatcgaaagcggcagcaacaccgtcagacgattctcctcgagaagcacccccgcgaggctagagggcccagttcccaggaccgttctggctcccgggtaggtcagcgctgcgacaacggggagagcgctctgcagggtgaagtagatcgggaacaggctattctggagcgtcgagaactgctgacggggaagcgtcctgaacgcgacggttccgccaacgaaggtctacatatataacggaatgttgtaggagcttttgatgagttatctatcctcctgaccaattgaccagaaacaaaactcaggatgtcaaacctgatagatctcgactcccagaagggtgccgtagctgattgaagtgtgatgtcagccgatctattagtagcagccgtagtactggtgacgcaccttagtatgtggaaggggcccaggatagacatcctgctctgttgtactgatatggaaacacctcgtgactggaacagaactatatgggattatacttagacagataccccactgactgggaattcagagggaagagtaagttgtgttatgctacgggtaggttagagaagctgtcaagcttgggtctcccgagctaacgctagctgcatgtggggcatgttcttatctccacggcccgctcaaacctagatctgcttccaacaaagcacaaatatctatacacacggccttttccgtaaggcccacgcaccttcccgacgtcatgtgcactcgcgtctgccgcgcctcaaaaaggaaatatcacgcgtctgcctggaggcgctccttagtcatagaaagaaacgcatctacgccatgcagtgatttatttatctgacatttccttcctcttcgttgcagcaggagggacagctgacatctcttttgcaaaatggctgacaaggaggccaccgtctacgtgatcgatgtgggaaagtccatggggaggcgccgccatggacggccggtatctgacctggaatgggcaatgcaatatgtctgggacaagattacgacaaccgtatgctgacacttgatccggtctcctggaaattaaattcctgcgttgagaactgacatatcttctgttaggttgccacggggcggaaaacggctacaattggagtggtcgggctgaggacagatggtgagattttaccgtgcccgaatcaggtaaatatgatttactgatgtatctggacagaaacatcgaacgacttgcaggatgatgacagctattcgcacatctctgtctttcaggaaattggacagtatgtgcctcagctgacactgatgactagtgacttttcctcgcatatactaaataaatcactgccagggtcctcatgcctgatctgcgaaaactgcgcgacctgatcaagcctagcaacactgatgaaggagatggtgagttttgcccgtatcttcggactcatttgatttgatattgagacctatctacctatagctatctcctcccttgtcgtcgcgatccagatgatcaccacttataccaaaaagctgaagtatcgacggaaaatcattctcgtgacgaacggggaaggatccatgagtaccgatggtcttgatgagatcgtgaaaaagctcaagtccgatagcattgaattggtggtcttgtatgtttttcacttctctttgacttttcttgtggctggtatgcaaaatggctaaactggtttcgttgcaggggtgttgactttgatgatcctgaatttggtgtcaaagaggaggacaagaatccagcaaaagtattcaatgttttttttttagcaggttggaagagttgctgattcgatctgccgcaggctgagaatgaagcggtcctcagaggtctcgttgattcctgcgacggagtctacgggacattacaacaggccatattggagctggacacaccgcgtgtgaaggttgttcgtggaataccctcctttagaggagagctccgactggggaaccctgaagagtattcgtctgcccttcgtatcccagtcgaaagatactaccgaacttatgttgccaagccgccgacagcgagctcctttgtcctacgatctgacgctgcagctggtcaagagggtgcagagaatgcactgacaagcgtccgaaacgcacggacatatcacgtcagtgatgagtccgcaccaggaggcaagagagacgtggagcgagaagatctcgccaagggctacgagtatgggagaaccgcggtgcacattagtgagtccgatgagaatatcaccaaactccagacgaaccctggtctggaaatcatcggcttcattcagagtgaccatgtatgtttctcgtcaagggtatctcatctgaaccgtgattaacctaggatccagtacgaccgatacatgcacatgtctaccagcaatgtcataattgcacagaaagcaaacgaaaaggcgatccttgctctttcatctttcattcacgccttgttcgagttggactgttatgctgtggccagacttgttaccaaggacaacaagcccccactcatcgtattactggcaccatctattgaagcagactttgaatgtcttctagaagtccagctcccttttgctgaagatgttcggtcgtaccgtttccctcccttggacaaggtggtcactgtctctggaaagacagtcaaagagcaccgacatctcccaagtgacgaattgctgaatgcgatgagcaaatacgtcgacagcatggagctcgtcgacaaggatgaaaacgggtgagtcatcacagggaaaccgtcatgctgctcatctcaagtatactgacaactccacagagaaccagttgacagcctggctcccagactggaggattcgtactctccactgctgcacaggatcgagcaagctatccggtggcgtgccatccatccaaacgagcctcttccgcccccttctgagaagttgacgcagctgtcacgaccgccagcagatctgcaagcgcgcgcgaagaaatacctggatcgggtcattgccgccgccgatgtgaagaaaggtctgtcaacttctacgctcccccagaatgcatctgactaaaaaatgctgcacagttccaccaaaagcaaaaggtcgcaagcggaatcgcgaagccgacaaacccctatcgggtcttgacgttgacgagctccttcgtcgcgagaagcgcgccaagatctcagccaacaacgccatccccgagttcaaacagtcgctggtcaacgccgagaccatcgacgccgtccgtgacgcagtcagccagatggaaagcatcatcgagaaccacatccgaagcagctttggagacgccaactacgaccgcgtgatcgaggagctgggtgtcctccgcgaggagctgatcgcctacgaagagccggatctctacaacgacttcctgcggaggctgaaggacaagatcctcaatgaggagctgggcggagacagacgagagctgtggtggctcgtcaggaggcaacgggtcggtctgatagacaagaaggcgtcggaacgggttgaagttactgaacaggaagccagggaggtaagtaagcagatacattattcctttagttccattaaacgagctgcatgatgagctgacttttgttcactagttcatgacctcgaaataaaatagtccattattgctatgtatgtcaaggcgcctggccgtagtagtcttaacatgctgatgctgtgaatcaaagcgccagatgaacaataatagaaataataccacttggtagctgtctccattctcacagatagacaacgttaaagaaaagaaaaacgtaaaaagagggtatatgtggtctagtaacgccgcaaggaaaaaaaaactcatacgttagtttcgaacgcaaatctcaaaatcgagcacttcgagtaaatactctgtcgtatcgtttcgcctcaggatatcttcccgagccttctctttccgatatcgattttccgttgtaatctagttattattactccagttagtaaatgcacgacgggcagtattgtaaataatgaaatcagcagcgagagtacgaacatgtccacatcctcatcggctttccggagcaactcgttctggatctccagctcattgttaatggcgatccccagctccttctgtcgagcgacgattttcatcaactcctccacgctccggtcctgctcttccatcgtctgcttctgcagctggagcacgccctggttgtcgagttcccgcgtcttgtccgtttccttgcccaggactcgtccagaacggggtttggcgctccccaccagggcgtccttgtcctgcatcgaagcgacagcgttgtcgagcttgctcttcgtcaccatcgcgttgtgcagattctccaatccgtccttctctttcttggcgctcgcgatgagatccttcctccgacggatctctccctcgcctaacctgctgccgccccatccagacgacttgtcgctcaggttcttcagcccttcctcaagagcgccgatcatcgaccctgctttcaccaagctgcttttggcctgtgccgagctctcgtgttgtttctgtggagtcgtggcctgatcacgtctcgtcagatgcagcctcgtctcgtgtaagtgcgccttcatctcccggtagcaatccagccacaggactggatccgtgatcggtccgccgcctggcgcgcccggttccgtgatggacttgtgaagcttcgatgcggcagagctatccgacagggcttgggaggggaggtttagaaaggacctccagacgcttgtttgacgccatcgcgggtcctcgctctcgttgatggcccgcaggtatctttccaggcctttgcgccgctcttcgcgcagagtctcgttcgagttcgtgttggaaaaccaggacttcccgggcagagcgacgggtggttgggcgccaacctggcggactagtgcgtcatggaacgatgcaaattctgaatagcgtttctggacaacgaacgaccgtagaggcagccggatggtgatgttgtatagcgtatacggactgggagcgtccgcgatggtggctgtcgggatggaaatttcgacattcggggccatgattatagttcagacgggaaaaagaacaaaacaaagagcaggcccttgttatcgaccaggaagcataattcccgccgcttctcttgcggtatctctgtcgttgcagagttggttgcagagtagtggagtcggccggcgggtggaaactcccgcaatgacgcaggcgccccatcttcttctgccaccgccgatctgtggcttagcttcttcttgtcaagactcgactccaccatcgcgactccaggcagcacgaatcgcacgattgccgaaaaactacaccgtactaggggaaggcctaattaatctattaccctagctaaaaatggggttgtcaaacttatcatatagccgtgcgacccgcccttggaggtcactagatccaacctgcgcacggcctggttacggttgatgggagctaaaattagaacgaaagatatactggcggtccgtccccgcgtctatccacaatccaaaactcgtatgcagagttatctacaggtcgatccaatcatgagtcctttgtgacatgtcgttgaatacatggtctcaatcgagtctgccgttcttacatgaccatcctcaccaagatcaatgtcccgtgattcgactgtcagccaagatacgtctcacctggccccatctctactgtcgacaacgtctgcctatactgtaggtgatcagaatacgcagtcccggggagtctactcgcgatggggtggttcatacgtcggctcctcgtcgacgttgtctctgggtccgtcggagagcgtcaatatagacgggagacgaaagttgctcttgatctatatccatggcttcatgggtgaagaagcgagcttccacaagttccctgctcatgtccataaccttgtcaccattgctctggccgagtcgcacgttgtgtattcgaaggtatatcctcgatacaaatcccgccgagcaatggacattgcacgtgatgatttcagtcgatggtgcgtttgcagactggcatatctctctttagagatcatcctagaaagaaacgcatgatactaagtgtcgaataggctatcaccgcatgagtcggaagatacagatgtgatcctactcggccacagcctgggtgggatcctagccgcagaggttgcgctgctcccatcagcccctgggagcaaggagatcttcgagcatcgtatcctgggactcatca

SEQ ID NO: 42 - DNA, 2145 bp, Rasamsonia emersonii
atggctgacaaggaggccaccgtctacgtgatcgatgtgggaaagtccatggggaggcgccgccatggacggccggtatctgacctggaatgggcaatgcaatatgtctgggacaagattacgacaaccgttgccacggggcggaaaacggctacaattggagtggtcgggctgaggacagatgaaacatcgaacgacttgcaggatgatgacagctattcgcacatctctgtctttcaggaaattggacaggtcctcatgcctgatctgcgaaaactgcgcgacctgatcaagcctagcaacactgatgaaggagatgctatctcctcccttgtcgtcgcgatccagatgatcaccacttataccaaaaagctgaagtatcgacggaaaatcattctcgtgacgaacggggaaggatccatgagtaccgatggtcttgatgagatcgtgaaaaagctcaagtccgatagcattgaattggtggtcttgggtgttgactttgatgatcctgaatttggtgtcaaagaggaggacaagaatccagcaaaagctgagaatgaagcggtcctcagaggtctcgttgattcctgcgacggagtctacgggacattacaacaggccatattggagctggacacaccgcgtgtgaaggttgttcgtggaataccctcctttagaggagagctccgactggggaaccctgaagagtattcgtctgcccttcgtatcccagtcgaaagatactaccgaacttatgttgccaagccgccgacagcgagctcctttgtcctacgatctgacgctgcagctggtcaagagggtgcagagaatgcactgacaagcgtccgaaacgcacggacatatcacgtcagtgatgagtccgcaccaggaggcaagagagacgtggagcgagaagatctcgccaagggctacgagtatgggagaaccgcggtgcacattagtgagtccgatgagaatatcaccaaactccagacgaaccctggtctggaaatcatcggcttcattcagagtgaccattacgaccgatacatgcacatgtctaccagcaatgtcataattgcacagaaagcaaacgaaaaggcgatccttgctctttcatctttcattcacgccttgttcgagttggactgttatgctgtggccagacttgttaccaaggacaacaagcccccactcatcgtattactggcaccatctattgaagcagactttgaatgtcttctagaagtccagctcccttttgctgaagatgttcggtcgtaccgtttccctcccttggacaaggtggtcactgtctctggaaagacagtcaaagagcaccgacatctcccaagtgacgaattgctgaatgcgatgagcaaatacgtcgacagcatggagctcgtcgacaaggatgaaaacggagaaccagttgacagcctggctcccagactggaggattcgtactctccactgctgcacaggatcgagcaagctatccggtggcgtgccatccatccaaacgagcctcttccgcccccttctgagaagttgacgcagctgtcacgaccgccagcagatctgcaagcgcgcgcgaagaaatacctggatcgggtcattgccgccgccgatgtgaagaaagttccaccaaaagcaaaaggtcgcaagcggaatcgcgaagccgacaaacccctatcgggtcttgacgttgacgagctccttcgtcgcgagaagcgcgccaagatctcagccaacaacgccatccccgagttcaaacagtcgctggtcaacgccgagaccatcgacgccgtccgtgacgcagtcagccagatggaaagcatcatcgagaaccacatccgaagcagctttggagacgccaactacgaccgcgtgatcgaggagctgggtgtcctccgcgaggagctgatcgcctacgaagagccggatctctacaacgacttcctgcggaggctgaaggacaagatcctcaatgaggagctgggcggagacagacgagagctgtggtggctcgtcaggaggcaacgggtcggtctgatagacaagaaggcgtcggaacgggttgaagttactgaacaggaagccagggagtccattattgctatctgtctccattctcacagatag

SEQ ID NO: 43 - protein, 714 aa, Rasamsonia emersonii
MADKEATVYVIDVGKSMGRRRHGRPVSDLEWAMQYVWDKITTTVATGRKTATIGVVGLRTDETSNDLQDDDSYSHISVFQEIGQVLMPDLRKLRDLIKPSNTDEGDAISSLVVAIQMITTYTKKLKYRRKIILVTNGEGSMSTDGLDEIVKKLKSDSIELVVLGVDFDDPEFGVKEEDKNPAKAENEAVLRGLVDSCDGVYGTLQQAILELDTPRVKVVRGIPSFRGELRLGNPEEYSSALRIPVERYYRTYVAKPPTASSFVLRSDAAAGQEGAENALTSVRNARTYHVSDESAPGGKRDVEREDLAKGYEYGRTAVHISESDENITKLQTNPGLEIIGFIQSDHYDRYMHMSTSNVIIAQKANEKAILALSSFIHALFELDCYAVARLVTKDNKPPLIVLLAPSIEADFECLLEVQLPFAEDVRSYRFPPLDKVVTVSGKTVKEHRHLPSDELLNAMSKYVDSMELVDKDENGEPVDSLAPRLEDSYSPLLHRIEQAIRWRAIHPNEPLPPPSEKLTQLSRPPADLQARAKKYLDRVIAAADVKKVPPKAKGRKRNREADKPLSGLDVDELLRREKRAKISANNAIPEFKQSLVNAETIDAVRDAVSQMESIIENHIRSSFGDANYDRVIEELGVLREELIAYEEPDLYNDFLRRLKDKILNEELGGDRRELWWLVRRQRVGLIDKKASERVEVTEQEARESIIAICLHSHR

SEQ ID NO: 44 - DNA, 4063 bp, Rasamsonia emersonii
ctcggagctgggaggcgtgctctcgttcggatccacggtttatggcttcgcgactggctggaccagctatgcagcagattacaccgtctatcagcccgctaaccgcagtcgccgcaaggtgtttttggcgacatggcttggtatcatcactcctctcctcttcacggagatgctgggcgtggctgtcatgaccgccaccagccttaatgacggcaacaatgcctaccaggacggctataacgcttctggcaccggtggcttgctggctgctgtcctcttccccaagctgggtggctttggcaagttctgcgtcgtcatcctcgcactgtcaatcattgccaacaactgccccaacatttactcggtgtcccttaccctcttagttctgggccgttggactcgtctcataccgcgcttcatctggactcttgtagccaccggggtctacgtcgccattgctatccccggctattcgcacttcggcggtgttggagaatttcatgaacttcatcgcttactggctggcgatctatgaggggattgccgtgaccgatcactttgtcttcaagcgtggtttctcagggtaccgacccgaaatctacgatcggcccgacgaactacccccgggcattgctgcggtgggtgcgttctgctgtggtgttgcgggaatgatcaccggaatgagccagcagtggtgggtcggtcccattgctctgcacgctggcgaagcgccgtttggcggcgacgtcggctttgagcttggctttgcattcgctgctgtcggctatttaatcttgagaccgatcgagctgaggatcttcaagcgatagggttgtttggatggcattgtgagggaaattggtgtcttcgtgttttttttttgatgaggttatatgtttatatatattcatctttcgtcatcttttagccgtcatggagtgtaattatgtcgataccatcaatgcatgtatattgagaggcatttcctttctgggaagtatagacctagtacacaatatagcgagtcgaacctgatatcagaggatctatcggtatatatcatcagacgttattcacatagtcagggaatatcatcccagcatcaaaggacattcaagttttctcagtaggttgtgatcaggtcctgctaacctggatatgtagagtacatatactttgagagttgagttcaatctcctcctcttttctcttttgaaagccaaattgaggtgtgctgctaaattgtctaataaatagaatcacacgacaagggagcactgcaagtacaacagttaaagagaatgcaaatgattaccatgcgtcataggacaagagtatagatctgcttggtcacgtgacttgttcgctatgtctagccacacggctgacctgcgccaccgattgaatccatctaaaagaaatccacaacggattcgcctccgctccgaaatcaagaaaattgatcacaatcaagaaaccacaatattatcaccacaaagatgtcgagactggcaaagtaagtacattatcaaatattccggacgtgtgctggacataattttgctaacattgtcttctttcatctacagccgtgccgactgggccgacgacgaggagtttgatgacccctccgccctccccgcacagcaagtcacaaccaacaaggacggaacgaaaactgtcgtgtcgtatcgcttcaacgatgatggcaaaaaagtgaaagtgacgcggcggatccgcaccaccgtcgtgaaggagcacgtcaaccctcgagtcgcggaacggagaacatgggccaagttcggtctggagaagggccatgctcccggcccatcgtttgacaccacctcggtcggcgagaacattgtcttccgtcccagcgtcaactggaaggcgcaggccaaggaggcggagaaggaaggcgagaagggcggcatcaaggaccagctgaaggacaagaaggtcaagtgccggatttgcagcggagagcacttcacggctcgctgtccgttcaaggacacgatggctccgatcgacgagcctgctcacggccccggagtggatttggacgacgacgacaggcccacgggcgctctgggcggtggtggaggaagctatgtgcctcctcacctgcgcaaaggcggtgctgcggctggcgagaagatgggtggcaagtacgagagagacgatcttgcgaccctgagagtgacaaacgtaagtgctatttgccagccttttctagatgagatatgagctgacacggattgcttctactcaggtgagcgagttggcagaagagcaggaactccgggatctattcgaacggtttggacgggtcacccgagtctttttggcccgggaccgcgacacgcagaaagccaagggctttgcatttatcagcttcgcagaccggaacgacgctgctcgtgcttgcgagaagatggatggctgtaagtgtccccacgatggtatcatgagtctcggtcatatgctgacattaactcctccagtcggttaccgccaccttatcctgcgcgtggaattcgccaagagggcttcttaaaagaacttccttcccttttcttttacttcttcgcctcacccgtcatggcttatttcatttcatggttgaagattgtttgtactgtacgacgaatcgaacgacctttcgaattcggtaggatagcttggtaatgatgatgcttttttttcatgttgaacgagggatcagataagcaaatagcatcttttgattcttctctctcgtcttgctgctcgagcgtacatacatagaatgaattgatatatccttatgtccgccatctctctctccctgctgttgactatggaataacaaacagaaaaagctgattcagatatcaaaaggctatatgtattgaaatggaccactgaacgggacatgatatcccacccaaaaaaagtgttcctttcgatcgtcgcccttctctcgacccgatttcctttccttacccttaccttcttgatccaaggagttcaaaaagctcaaaagctcaagtgaaaagaaaagagttatttctactcaaactgtactgtacatatagagtagcttttattccttgtttctttttttgtcttgtgtgcgtttacatcagatatatgtacatacatacatatacatgcatatacacgaatcctcgctatactatagttgatacggatgtatatcctatcctatcttcttacttacatacataggaaagggaagtgaaaagagataccagaatacagaataaacaacagagaatgaatgagtgagtgggtgagtaagtgagtgaatttctacgaactgatcctcgtcaagtacagtaagacgagatgtaaatgaatatcaatctacttactccatgagtcagtcagtctattaagtatcacctctactatcacctctactataggtaaaaacattaatttccgtttttgtatactgtcatgtgcctctctaaatatctaaatattagggaagggatatatagctacagtagagttagggtaaaaaagagattacagcaggggatatagttagttagtaagtccggctagagagtagatgatgacaaggactaactactaaaatatgagaaataataataataataatatgtatatagtgaggcccgcctgggaggtggattgcaaagtccttggcataaccaggtatacctattttctcttcaaatcagtcgagcaaaatgagacctggaaggcttgtgtattatattataggtaatcaacagagattcattttgtatttgtttcattttattccgtctcatgcacgacagacaagcaacttttgcttgctacctacctactctgtaaagaaaactccatacatgacttcatgatatatgtggtatgcctgtcctgtcctgtccagatctatatctagatgagatgagatatatggttagtatgtacatacatacatgtactccgtaggtaactactaaagtaactacctaggtatactgtaagtcggagggcctgcagcctgccctggcggattgttcatgaatgcagt

SEQ ID NO: 45 - DNA, 927 bp, Rasamsonia emersonii
atgtcgagactggcaaaccgtgccgactgggccgacgacgaggagtttgatgacccctccgccctccccgcacagcaagtcacaaccaacaaggacggaacgaaaactgtcgtgtcgtatcgcttcaacgatgatggcaaaaaagtgaaagtgacgcggcggatccgcaccaccgtcgtgaaggagcacgtcaaccctcgagtcgcggaacggagaacatgggccaagttcggtctggagaagggccatgctcccggcccatcgtttgacaccacctcggtcggcgagaacattgtcttccgtcccagcgtcaactggaaggcgcaggccaaggaggcggagaaggaaggcgagaagggcggcatcaaggaccagctgaaggacaagaaggtcaagtgccggatttgcagcggagagcacttcacggctcgctgtccgttcaaggacacgatggctccgatcgacgagcctgctcacggccccggagtggatttggacgacgacgacaggcccacgggcgctctgggcggtggtggaggaagctatgtgcctcctcacctgcgcaaaggcggtgctgcggctggcgagaagatgggtggcaagtacgagagagacgatcttgcgaccctgagagtgacaaacgtgagcgagttggcagaagagcaggaactccgggatctattcgaacggtttggacgggtcacccgagtctttttggcccgggaccgcgacacgcagaaagccaagggctttgcatttatcagcttcgcagaccggaacgacgctgctcgtgcttgcgagaagatggatggctgtaagtgtccccacgatggtatcatgagtctcggtcatatgctgacattaactcctccagtcggttaccgccaccttatcctgcgcgtggaattcgccaagagggcttcttaa

SEQ ID NO: 46 - protein, 308 aa, Rasamsonia emersonii
MSRLANRADWADDEEFDDPSALPAQQVTTNKDGTKTVVSYRFNDDGKKVKVTRRIRTTVVKEHVNPRVAERRTWAKFGLEKGHAPGPSFDTTSVGENIVFRPSVNWKAQAKEAEKEGEKGGIKDQLKDKKVKCRICSGEHFTARCPFKDTMAPIDEPAHGPGVDLDDDDRPTGALGGGGGSYVPPHLRKGGAAAGEKMGGKYERDDLATLRVTNVSELAEEQELRDLFERFGRVTRVFLARDRDTQKAKGFAFISFADRNDAARACEKMDGCKCPHDGIMSLGHMLTLTPPVGYRHLILRVEFAKRAS

SEQ ID NO: 47 - DNA, 55 bp, Penicillium chrysogenum
ggggacaactttgtatagaaaagttgggcccaacgcatgtgtacgagagtcaagg

SEQ ID NO: 48 - DNA, 50 bp, Penicillium chrysogenum
ggggactgcttttttgtacaaacttgagacggaaggagatcgcgtaacag

SEQ ID NO: 49 - DNA, 50 bp, Penicillium chrysogenum
ggggacagctttcttgtacaaagtggggcgcagtccattcttgcatctac

SEQ ID NO: 50 - DNA, 53 bp, Penicillium chrysogenum
ggggacaactttgtataataaagttgggcccagccacttcttgtatcacggat

SEQ ID NO: 51 - DNA, 35 bp, Aspergillus nidulans
agagaggatccgagttggccagttgacaacctgag

SEQ ID NO: 52 - DNA, 37 bp, Aspergillus nidulans
agagagcggccgcgagtatgagcgatcgacacgaatg

SEQ ID NO: 53 - DNA, 3438 bp, Aspergillus nidulans
agagaggatccgagttggccagttgacaacctgagcatctcggagtttttttcacgggagcttgggccttgtggatgtccgatttccgagctgaggccgaggccgccttcaacagctgagctgatgtcaggccatcctgatattgttgaatgcgcttccgcggttaccggtcgtatcctgatggcgacaattttcgactcggtagcgccggcttgaatggtagaatgcagccgctcgaaaaaaagatggctgggaatcgttctttgttagatactcaacgtctcaccatatcgatagctctagtcgagtatccaggagctggtaggagctgggtggtgaactagactgcaagttaaatgaagacagtcaggggactgggtcttggcggtagggacccatttggttggccagcgccagcagatctgtggcttccggttggctacttgtaaccaactgatggtcagatggatctgccgtctgttttgatttgaattttccctgctcattctgattctgtgagaggctgcattcattatcacatctcatacccggcgcctgcgacttcggtcacctctgcggtctggcggttagcggggtgcgtctgagactcgtcagtcagcattcgagtatgcgaactctgactttgctcacctaagagtttgcacgagatgccgaaatcctcctcgagtagagtttgcaaggcttgaaccttggtccttgaagcccgaaagtggctcagtagtgggatcgatagtctggttgttgaagattttctcctccaccttacctatggccgctggccttctccacctttcaggctttcaggcaccctcggctcggattctgtatcgtccggtaccgaagctagtcctagctagtcaaagctagtccaagctagtctcgtcaaggtttggcgcagcgcggttccgtgtaaagtacaaatttgaaatacgaatacgcagtactcgcagccggcacttccgctcagcccaggctcagaggctaagggtgttggcgcttcctcatcatcttcttctcgtcgaccttttcctctttctctccctatcggtgcttctctccaacctcattctcagtcgttcgcccatcaggtttatactccggctccgtggccatctgcctccctcacgacctcctcgttccaggttttcctctcgactgctgcgcccttgcacttcgccttgcatcagtgaaaccccctgcaacgtgacggctcaaagacatcctcgtttggccgctggagaccggagcgtgcgcttcgtttcgtcttcttcgaaccgatctcaatttccccgctcgggttgacgccgtcagcaccctgctcgttgcctaacggcttgttattcaagaccccttttctgccgcttccgcgaccgatttattcgtcgccttccaactcttgtacaatcggggggaaagaaagcagacggagttcgatctggaggaattatagctgagtcttgcccgcaagactcgccgcaaccatgaatcaaacacttcccacgtggaaggaccgcacgcagaaccagtttggaaagcttcagatccaggttccatggcggtccatccaactgctcgtcccgcatcgcatgcggcggaagttaaggtccaaattgcgcagtagagcgtctcctacctcgtcaatagcctctttacagacgtcgttatcgcctgcagacacactacgatcgctccaaagccaccgatggacggtttacgacttccaatatctgcttctgttgatcgtgggcatcttctctttgaccgttatcgagtcgcccgggcctttgggcaaaacggccattttctccatgctcctattctctctcctgatccctatgacccgccagttcttcctcccgtttctgccgattgccggatggcttctgtttttctacgcctgccagtgagttaaaaacaacccgctaccagaccccgtgcagcagttactcacatatgcaggttcatcccaagcgattggcgccctgcgatttgggttcgtgtcttgcctgcactggagaatattctctacggcgcaaacatcagcaacatcctatccgctcaccagaacgttgtgcttgacgtgctggcgtggctaccctacggtatctgccactatggcgctccgtttgtgtgctcgttgatcatgttcatcttcggtccgcccggcactgttccccttttcgcgcgcactttcggctatatcagtatgactgcggttactattcagctgtttttcccttgctctccaccttggtatgagaatcgctatggtctagctccggcagactactccatccaaggtgatcccgcagggcttgcccgcattgacaagcttttcggcatcgacctttacacgtctggtttccatcagtcgcctgttgtgttcggcgcttttccgtcgctgcatgctgccgactcaaccctggccgcacttttcatgagtcatgttttcccccgcatgaagcccgtcttcgtgacctatactctatggatgtggtgggcaacaatgtacctctcacatcactatgcggtcgatttggttgcgggtggtctcctggccgccattgctttctacttcgccaagacccgattccttccccgtgtccagctcgacaagaccttccgttgggactacgactatgtggaattcggcgagtctgccctggagtatgggtatggtgcagctggctatgatggagacttcaatctcgacagcgatgaatggactgttggttcttcatcctccgtctcctcaggctccttgagtcccgttgacgatcattactcatgggaaaccgaggcactgacctccccacatactgatattgagtccggcaggcatactttcagcccttgagtagccacaaaccaaactcgatacctgcatatagcgatctcgctcctcctccactgcatctatttacgagacggcgttagaacatttcacgacattctggctttattgcatcgagcacatttcgacacatatatctttaataccctttcttcggtgtcccagatcatcggttcgaccttaatgtacctcggtccgaatccgcctgggatactgtttctctttccgccgcacttcactgtacattgcttgacattgcgaaaccgggttgggctcgaacgtgggatgggttatcgctcatcgctacacgccgttgctccatcataatgttaatggacacaatggggctacgcatcctggtgtttagtcctggaagaccatccgataacccccgtcggtaacactcgcttgtctcgtgtccacccagacactacttcaattctcacttctatcgtccgctattaccttgacctggtcgaacccatccttattattcgtttcgactatgctatatatttatttttaccattcgtgtcgatcgctcatactcgcggccgctctct

SEQ ID NO: 54 - DNA, 24 bp, Penicillium chrysogenum
agctttgacgctagattggagatg

SEQ ID NO: 55 - DNA, 24 bp, Penicillium chrysogenum
caagcaagccatctcaacaagtgc

SEQ ID NO: 56 - DNA, 70 bp, Saccharomyces cerevisiae
tactcgctgtattgaaaggatcaaaagaccaaagaccaccaggaataatgccagctgaagcttcgtacgc

SEQ ID NO: 57 - DNA, 73 bp, Saccharomyces cerevisiae
atgagaagagtaacattagaaaacaagtgcagagcatattctgtgcatctagcataggccactagtggatctg

SEQ ID NO: 58 - DNA, 1715 bp, Artificial sequence
  tif35::loxP-KanMX4-loxP deletion cassette
tactcgctgtattgaaaggatcaaaagaccaaagaccaccaggaataatgccagctgaagcttcgtacgctgcaggtcgacaacccttaatataacttcgtataatgtatgctatacgaagttattaggtctagagatctgtttagcttgcctcgtccccgccgggtcacccggccagcgacatggaggcccagaataccctccttgacagtcttgacgtgcgcagctcaggggcatgatgtgactgtcgcccgtacatttagcccatacatccccatgtataatcatttgcatccatacattttgatggccgcacggcgcgaagcaaaaattacggctcctcgctgcagacctgcgagcagggaaacgctcccctcacagacgcgttgaattgtccccacgccgcgcccctgtagagaaatataaaaggttaggatttgccactgaggttcttctttcatatacttccttttaaaatcttgctaggatacagttctcacatcacatccgaacataaacaaccatgggtaaggaaaagactcacgtttcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggcaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagtactgacaataaaaagattcttgttttcaagaacttgtcatttgtatagtttttttatattgtagttgttctattttaatcaaatgttagcgtgatttatattttttttcgcctcgacatcatctgcccagatgcgaagttaagtgcgcagaaagtaatatcatgcgtcaatcgtatgtgaatgctggtcgctatactgctgtcgattcgatactaacgccgccatccagtgtcgaaaacgagctctcgagaacccttaatataacttcgtataatgtatgctatacgaagttattaggtgatatcagatccactagtggcctatgctagatgcacagaatatgctctgcacttgttttctaatgttactcttctcat

SEQ ID NO: 59 - DNA, 40 bp, Saccharomyces cerevisiae
gaataaaaaagagctcacgctttttcagttcgagtttatc

SEQ ID NO: 60 - DNA, 41 bp, Saccharomyces cerevisiae
gttaatagcaactctaaccatggtttgtttgtttatgtgtg

SEQ ID NO: 61 - DNA, 26 bp, Saccharomyces cerevisiae
gcttatggtggtggtgcttcttatag

SEQ ID NO: 62 - DNA, 35 bp, Saccharomyces cerevisiae
agagggtacctgtgcatctattccttaaccttagg

SEQ ID NO: 63 - DNA, 24 bp, Saccharomyces cerevisiae
agtacggtcattggacctggaatc

SEQ ID NO: 64 - DNA, 24 bp, Saccharomyces cerevisiae
cgttccatgcacctccatgaatgt

SEQ ID NO: 65 - DNA, 20 bp, Penicillium chrysogenum
ccaccgttgtccgcgaacat

SEQ ID NO: 66 - DNA, 20 bp, Penicillium chrysogenum
tccttctcggcctccttagc

SEQ ID NO: 67 - DNA, 20 bp, Penicillium chrysogenum
ctggcggtatccacgtcacc

SEQ ID NO: 68 - DNA, 20 bp, Penicillium chrysogenum
aggccagaatggatccaccg

SEQ ID NO: 69 - DNA, 20 bp, Aspergillus nidulans
ggctggctgttagtcaactg

SEQ ID NO: 70 - DNA, 20 bp, Aspergillus nidulans
aggaggctgacctcgattgt